SEQUENCE LISTING (22 sequences)

SEQ ID NO: 1 - DNA, 18 bp, artificial sequence
  synthetic oligonucleotide
tggagcaagtgcctcctc

SEQ ID NO: 2 - DNA, 21 bp, artificial sequence
  synthetic oligonucleotide
atattaccacttcgattggtc

SEQ ID NO: 3 - DNA, 21 bp, artificial sequence
  synthetic oligonucleotide
tggagctggaagtgcctcctc

SEQ ID NO: 4 - DNA, 21 bp, artificial sequence
  synthetic oligonucleotide
gtgatgggagagtccgataga

SEQ ID NO: 5 - DNA, 21 bp, artificial sequence
  synthetic oligonucleotide
gtcagctggggtcgtttcatc

SEQ ID NO: 6 - DNA, 21 bp, artificial sequence
  synthetic oligonucleotide
ctggttggcctgccctatata

SEQ ID NO: 7 - DNA, 21 bp, artificial sequence
  synthetic oligonucleotide
gtcagctggggtcgtttcatc

SEQ ID NO: 8 - DNA, 21 bp, artificial sequence
  synthetic oligonucleotide
gtgaaaggatatcccaataga

SEQ ID NO: 9 - DNA, 21 bp, artificial sequence
  synthetic oligonucleotide
gtagtcccggcctgcgtgcta

SEQ ID NO: 10 - DNA, 21 bp, artificial sequence
  synthetic oligonucleotide
gaccggttacacagcctcgcc

SEQ ID NO: 11 - DNA, 21 bp, artificial sequence
  synthetic oligonucleotide
gtagtcccggcctgcgtgcta

SEQ ID NO: 12 - DNA, 21 bp, artificial sequence
  synthetic oligonucleotide
tccttgcacaatgtcaccttt

SEQ ID NO: 13 - DNA, 21 bp, artificial sequence
  synthetic oligonucleotide
ccctgccgggatcgtgacccg

SEQ ID NO: 14 - DNA, 21 bp, artificial sequence
  synthetic oligonucleotide
tcgaagccgcggctgccaaag

SEQ ID NO: 15 - DNA, 21 bp, artificial sequence
  synthetic oligonucleotide
cggacactcccgggaggtagt

SEQ ID NO: 16 - DNA, 18 bp, artificial sequence
  synthetic oligonucleotide
cttctgtcgagtagggga

SEQ ID NO: 17 - DNA, 21 bp, artificial sequence
  synthetic oligonucleotide
tgcgggccagggacggagaag

SEQ ID NO: 18 - DNA, 21 bp, artificial sequence
  synthetic oligonucleotide
ctagttactactttggatagt

SEQ ID NO: 19 - DNA, 21 bp, artificial sequence
  synthetic oligonucleotide
cgggcgctgcgctgaaccggc

SEQ ID NO: 20 - DNA, 21 bp, artificial sequence
  synthetic oligonucleotide
tcgacatggggttcttcagtg

SEQ ID NO: 21 - DNA, 25 bp, artificial sequence
  synthetic oligonucleotide
gccagctcaccatggatgatgatat

SEQ ID NO: 22 - DNA, 25 bp, artificial sequence
  synthetic oligonucleotide
gcttgctgatccacatctgctggaa

(19) United States
(12) Patent Application Publication
(10) Pub. No.: US 2009/0105463 A1
(43) Pub. Date: Apr. 23, 2009

(54) Compositions of and Methods of Using Oversulfated Glycosaminoglycans

(75) Inventors: David A. Berry, Brookline, MA (US); Kevin Pojasek, Cambridge, MA (US); Dongfang Liu, Yorktown Heights, NY (US); Chi-Pong Kwan, Framingham, MA (US); Yiwei Qi, Andover, MA (US); Ram Sasisekharan, Bedford, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 11/887,559

(22) PCT Filed: Mar. 29, 2006
(86) PCT No.: PCT/US06/11674

§ 371 Date: Dec. 15, 2008

Related U.S. Application Data
(60) Provisional application No. 60/666,743, filed on Mar. 29, 2005.

(51) Int. Cl. C07K 14/475

(52) U.S. Cl. 530/399

(57) ABSTRACT

The invention relates, in part, to compositions comprising glycosaminoglycans, fragments of glycosaminoglycans or glycosaminoglycan fractions. The compositions provided can be used in various methods of modulating FGF and/or VEGF activity. The method can be in vitro or in vivo methods. Therefore, the invention also relates, in part, to methods of treating a subject with the compositions provided.

### RELATED APPLICATIONS

This application claims priority under 35 U.S.C. §119 from U.S. provisional application Ser. No. 60/666,743, filed Mar. 29, 2005. The entire contents of which is herein incorporated by reference.

### GOVERNMENT SUPPORT

Aspects of the invention may have been made using funding from National Institutes of Health Grant numbers HL-59966 and CA-90940. Accordingly, the government may have rights in the invention.

### FIELD OF THE INVENTION

The invention relates, in part, to compositions comprising glycosaminoglycans, fragments of glycosaminoglycans or glycosaminoglycan fractions. The compositions provided can be used in various methods of modulating FGF and/or VEGF activity. The method can be in vitro or in vivo methods. Therefore, the invention also relates, in part, to methods of treating a subject with the compositions provided.

### BACKGROUND OF THE INVENTION

Glycosaminoglycans (GAGs) are important regulators of biological functions. All GAGs are linear polysaccharides composed a disaccharide repeat unit that contains uronic acid and a hexosamine, where the specific nature of each defines the class of GAG [427]. The heparin/heparan sulfate-like glycosaminoglycans (HSGAGs) are the best studied of the glycosaminoglycans. The five sites of variation in the HSGAG disaccharide allow for enormous structural heterogeneity that enables them to modulate a wide range of important biological processes including development and tumor progression [38, 427]. HSGAGs interact with all known members of the fibroblast growth factor (FGF) family [392]. Other GAGs, such as dermatan sulfate (DS) and chondroitin sulfate (CS) have also emerged as important regulators of biological processes including FGF-mediated activity [474].

The FGF protein family consists of at least 23 members. Each FGF interacts with at least one of five high affinity cell surface tyrosine kinase receptors [119, 445] and with the GAG component of proteoglycans [153, 178, 396]. While HSGAGs interact with all known FGFs, the structural requirement of a HSGAG to promote a cellular response differs based on the FGF [213, 392, 512]. Fibroblast growth factor receptor (FGFR) isoforms support cellular activity downstream only of specific FGF family members [348]. HSGAGs interact with both the FGF and the FGFR to provide receptor selectivity and to regulate the cellular response [6, 213, 354]. FGF7 induces a downstream response through FGFR2b [124, 348]. The magnitude of cellular response to FGF7 can be regulated by HSGAGs as well as DS [475, 512]. HSGAGs and DS regulate FGF2-mediated activity through FGFR1c, while only HSGAGs have been shown to regulate that of FGF1 [366, 475].

Vascular endothelial growth factor (VEGF) is a major regulator of angiogenesis and cell growth [485]. VEGF isoforms show variable interactions with HSGAGs [400]. VEGF signals through the tyrosine kinases vascular endothelial growth factor receptor (VEGFR)-1 and VEGFR2, which are predominantly, but not exclusively, found on endothelial cells [201, 400]. VEGF-C and VEGF-D signal through VEGFR2 and VEGFR3 [2, 209]. VEGFR3 activity is associated with lymphangiogenesis [249]. VEGF-D, but not VEGF, promotes the lymphatic spread of tumors [450]. While the dependence of VEGF on HS GAGs has been established [196], the interactions of VEGF-C and VEGF-D with HSGAGs and other GAGs have not been determined.

The ability of HSGAGs, DS and other GAGs to modulate FGFs and vascular endothelial growth factors (VEGFs) is important in several physiological and pathological settings. FGF7 signaling through FGFR2b is important in wound healing, for example [203]. DS derived from wound fluid promotes FGF7 activity through its receptor [475]. VEGFR3 is also upregulated during wound healing, where it promotes angiogenesis downstream of VEGF-C and VEGF-D [357]. FGF, VEGF and various GAGs have also been implicated in cancer growth and progression [196, 427], promoting not only angiogenesis, but also primary tumor growth directly, such as in prostate cancer [201, 356]. FGF and VEGF can activate similar pathways to produce a common biological outcome, though the activity of one ligand may be dependent on the activity of the other [249, 390]. Understanding the ability of GAGs to differentially interact with various FGFs and VEGFs, both individually and in the same cellular environment, can shed insight into the role of each of these components in biologically important settings.

### SUMMARY OF THE INVENTION

Aspects of the invention relate to methods of modulating an activity of a fibroblast growth factor (FGF), comprising contacting the FGF with a composition comprising a highly sulfated glycosaminoglycan (GAG). In one embodiment, the highly sulfated GAG is in an amount effective to modulate the activity of the FGF. In yet another embodiment, the highly sulfated GAG is a highly sulfated chondroitin sulfate (CS) or a highly sulfated dermatan sulfate (DS). In one embodiment, the highly sulfated GAG is an oversulfated dermatan sulfate (DS). In another embodiment, at least 40% of the disaccharides of the oversulfated DS are either di- or tri-sulfated. In another embodiment, at least 50% of the disaccharides of the oversulfated DS are either di- or tri-sulfated. In a further embodiment, at least 60% of the disaccharides of the oversulfated DS are either di- or tri-sulfated. In another embodiment, at least 70% of the disaccharides of the oversulfated DS are either di- or tri-sulfated. In yet another embodiment, at least 80% of the disaccharides of the oversulfated DS are either di- or tri-sulfated.

In another embodiment of the invention, the highly sulfated GAG is a highly sulfated chondroitin sulfate (CS). In one embodiment, at least 40% of the disaccharides of the highly sulfated CS are either di- or tri-sulfated. In another embodiment, at least 50% of the disaccharides of the highly sulfated CS are either di- or tri-sulfated. In a further embodiment, at least 60% of the disaccharides of the highly sulfated CS are either di- or tri-sulfated. In another embodiment, at least 70% of the disaccharides of the highly sulfated CS are either di- or tri-sulfated. In yet another embodiment, at least 80% of the disaccharides of the highly sulfated CS are either di- or tri-sulfated. In still another embodiment of the invention, the highly sulfated CS is chondroitin sulfate D or chondroitin sulfate E.

In one embodiment, the FGF is FGF1, FGF2 or FGF7. In another embodiment, the activity of the FGF is increased. In a further embodiment, the activity of a vascular endothelial growth factor (VEGF) is also modulated. In another embodiment, the activity of the VEGF is increased.

In another embodiment of the invention, the composition is administered to a subject. In one embodiment, the subject has a wound, scar, chronic liver disease, benign hyperplastic hypertrophy, cancer or an inflammatory disease. In another embodiment, the composition further comprises a pharmaceutically acceptable carrier. In a further embodiment, the composition further comprises an additional therapeutic agent. In another embodiment, the additional therapeutic agent is an anti-cancer agent or an anti-inflammatory agent. In a further embodiment, the additional therapeutic agent is a FGF and/or VEGF.

In another aspect of the invention a method of treating a subject is provided. Such a method includes the step of administering to a subject in need of such a treatment a compositions of a highly sulfated GAG. In one embodiment the highly sulfated GAG is a highly sulfated CS or a highly sulfated DS. In another embodiment, the subject has a wound, scar, chronic liver disease, benign hyperplastic hypertrophy, cancer or an inflammatory disease. In still another embodiment, the composition further comprises a pharmaceutically acceptable carrier. In a further embodiment, the composition further comprises an additional therapeutic agent. In another embodiment, the additional therapeutic agent is an anti-cancer agent or an anti-inflammatory agent. In a further embodiment, the additional therapeutic agent is a FGF and/or VEGF.

In an embodiment of the invention, the method further comprises determining the presence or absence of the FGF in the subject. In another embodiment, the method further comprises determining the presence or absence of a VEGF in the subject. In another embodiment, the VEGF is VEGF-A, VEGF-C or VEGF-D. In a further embodiment, the VEGF is VEGF₁₂₀, VEGF₁₆₄ or VEGF₁₈₈. In another embodiment, the VEGF is VEGF₁₂₁, VEGF₁₄₅, VEGF₁₆₅, VEGF₁₈₉ or VEGF₂₀₆. In yet another embodiment, the determining step is performed prior to the contacting step.

Aspects of the invention relate to methods of modulating an activity of a FGF, comprising contacting the FGF with a composition comprising GAGs of a highly sulfated GAG fraction. In one embodiment, the GAGs of a highly sulfated GAG fraction are in an amount effective to modulate the activity of the FGF. In another embodiment, the highly sulfated GAG fraction is a highly sulfated DS fraction or a highly sulfated CS fraction. In an embodiment, at least 70% of the dermatan sulfates or chondroitin sulfates of the highly sulfated GAG fraction are highly sulfated. In another embodiment, at least 80% of the dermatan sulfates or chondroitin sulfates of the highly sulfated GAG fraction are highly sulfated. In another embodiment, at least 90% of the dermatan sulfates or chondroitin sulfates of the highly sulfated GAG fraction are highly sulfated.

In an embodiment of the invention, the FGF is FGF1, FGF2 or FGF7. In another embodiment, the activity of the FGF is increased. In another embodiment, the activity of a VEGF is also modulated. In a further embodiment, the activity of the VEGF is increased.

In an embodiment of the invention, the composition is administered to a subject. In another embodiment, the subject has a wound, scar, chronic liver disease, benign hyperplastic hypertrophy, cancer or an inflammatory disease. In another embodiment, the composition further comprises a pharmaceutically acceptable carrier. In a further embodiment, the composition further comprises an additional therapeutic agent. In another embodiment, the additional therapeutic agent is an anti-cancer agent or an anti-inflammatory agent. In a further embodiment, the additional therapeutic agent is a FGF and/or VEGF.

In another aspect of the invention a method of treating a subject is provided. Such a method includes the step of administering to a subject in need of such a treatment a compositions comprising GAGs of a highly sulfated GAG fraction. In one embodiment the highly sulfated GAG fraction is a highly sulfated CS fraction or a highly sulfated DS fraction. In another embodiment, the subject has a wound, scar, chronic liver disease, benign hyperplastic hypertrophy, cancer or an inflammatory disease. In still another embodiment, the composition further comprises a pharmaceutically acceptable carrier. In a further embodiment, the composition further comprises an additional therapeutic agent. In another embodiment, the additional therapeutic agent is an anti-cancer agent or an anti-inflammatory agent. In a further embodiment, the additional therapeutic agent is a FGF and/or VEGF.

In an embodiment of the invention, the method further comprises determining the presence or absence of the FGF in the subject. In another embodiment, the method further comprises determining the presence or absence of a VEGF in the subject. In another embodiment, the VEGF is VEGF-A, VEGF-C or VEGF-D. In a further embodiment, the VEGF is VEGF₁₂₀, VEGF₁₆₄ or VEGF₁₈₈. In another embodiment, the VEGF is VEGF₁₂₁, VEGF₁₄₅, VEGF₁₆₅, VEGF₁₈₉ or VEGF₂₀₆. In yet another embodiment, the determining step is performed prior to the contacting step.

Aspects of the invention relate to methods of modulating an activity of a VEGF, comprising contacting the VEGF with a composition comprising a highly sulfated GAG. In one embodiment, the highly sulfated GAG is in an amount effective to modulate the activity of the VEGF. In another embodiment, the highly sulfated GAG is a highly sulfated CS or a highly sulfated DS. In an embodiment, the highly sulfated GAG is an oversulfated DS. In another embodiment, at least 40% of the disaccharides of the oversulfated dermatan sulfate are either di- or tri-sulfated. In another embodiment, at least 50% of the disaccharides of the oversulfated dermatan sulfate are either di- or tri-sulfated. In a further embodiment, at least 60% of the disaccharides of the oversulfated dermatan sulfate are either di- or tri-sulfated. In another embodiment, at least 70% of the disaccharides of the oversulfated dermatan sulfate are either di- or tri-sulfated. In yet another embodiment, at least 80% of the disaccharides of the oversulfated dermatan sulfate are either di- or tri-sulfated.

In an embodiment of the invention, the highly sulfated GAG is a highly sulfated CS. In another embodiment, at least 40% of the disaccharides of the highly sulfated chondroitin sulfate are either di- or tri-sulfated. In another embodiment, at least 50% of the disaccharides of the highly sulfated chondroitin sulfate are either di- or tri-sulfated. In a further embodiment, at least 60% of the disaccharides of the highly sulfated chondroitin sulfate are either di- or tri-sulfated. In another embodiment, at least 70% of the disaccharides of the highly sulfated chondroitin sulfate are either di- or tri-sulfated. In yet another embodiment, at least 80% of the disaccharides of the highly sulfated chondroitin sulfate are either di- or tri-sulfated. In still another embodiment, the highly sulfated CS is chondroitin sulfate D or chondroitin sulfate E.

In an embodiment of the invention, the VEGF is VEGF-A, VEGF-C or VEGF-D. In another embodiment, the VEGF is VEGF₁₂₀, VEGF₁₆₄ or VEGF₁₈₈. In another embodiment, the VEGF is VEGF₁₂₁, VEGF₁₄₅, VEGF₁₆₅, VEGF₁₈₉ or VEGF₂₀₆. In a further embodiment, the activity of the VEGF is increased. In another embodiment, the activity of a FGF is also modulated. In yet another embodiment, the activity of the FGF is increased.

In an embodiment of the invention, the composition is administered to a subject. In another embodiment, the subject has a wound, scar, chronic liver disease, benign hyperplastic hypertrophy, cancer or an inflammatory disease. In another embodiment, the subject has a disease associated with excessive VEGF-mediated angiogenesis, In a further embodiment, the disease associated with excessive VEGF-mediated angiogenesis is age-related macular degeneration (AMD) or diabetic neuropathy. In another embodiment, the subject is in need of angiogenesis inhibition. In yet another embodiment, the composition further comprises a pharmaceutically acceptable carrier. In a further embodiment, the composition further comprises an additional therapeutic agent. In still a further embodiment, the additional therapeutic agent is an anti-cancer agent or an anti-inflammatory agent. In a further embodiment, the additional therapeutic agent is a FGF and/or VEGF. In another embodiment, the method further comprises determining the presence or absence of the VEGF in the subject. In yet another embodiment, the method further comprises determining the presence or absence of a FGF in the subject. In still another embodiment, the FGF is FGF7. In a further embodiment, the determining step is performed prior to the contacting step.

In another aspect of the invention a method of treating a subject is provided, wherein the method includes the step of administering to a subject in need of such treatment a composition comprising a highly sulfated GAG, wherein the highly sulfated GAG is administered in an amount effective to modulate an activity of a VEGF. In one embodiment, the highly sulfated GAG is a highly sulfated CS or a highly sulfated DS. In another embodiment, the subject has a wound, scar, chronic liver disease, benign hyperplastic hypertrophy, cancer or an inflammatory disease. In another embodiment, the subject has a disease associated with excessive VEGF-mediated angiogenesis. In a further embodiment, the disease associated with excessive VEGF-mediated angiogenesis is age-related macular degeneration (AMD) or diabetic neuropathy. In another embodiment, the subject is in need of angiogenesis inhibition. In yet another embodiment, the composition further comprises a pharmaceutically acceptable carrier. In a further embodiment, the composition further comprises an additional therapeutic agent. In still a further embodiment, the additional therapeutic agent is an anti-cancer agent or an anti-inflammatory agent. In a further embodiment, the additional therapeutic agent is a FGF and/or VEGF. In another embodiment, the method further comprises determining the presence or absence of the VEGF in the subject. In yet another embodiment, the method further comprises determining the presence or absence of a FGF in the subject. In still another embodiment, the FGF is FGF7. In a further embodiment, the determining step is performed prior to the contacting step.

Aspects of the invention relate to methods of modulating an activity of a VEGF, comprising contacting the VEGF with a composition comprising GAGs of a highly sulfated GAG fraction. In one embodiment, the GAGs of a highly sulfated GAG fraction are in an amount effective to modulate the activity of the VEGF. In another embodiment, the highly sulfated GAG fraction is a highly sulfated DS fraction or a highly sulfated CS fraction. In an embodiment, at least 70% of the dermatan sulfates or chondroitin sulfates of the highly sulfated GAG fraction are highly sulfated. In another embodiment, at least 80% of the dermatan sulfates or chondroitin sulfates of the highly sulfated GAG fraction are highly sulfated. In another embodiment, at least 90% of the dermatan sulfates or chondroitin sulfates of the highly sulfated GAG fraction are highly sulfated.

In an embodiment of the invention, the VEGF is VEGF-A, VEGF-C or VEGF-D. In another embodiment, the VEGF is VEGF₁₂₀, VEGF₁₆₄ or VEGF₁₈₈. In another embodiment, the VEGF is VEGF₁₂₁, VEGF₁₄₅, VEGF₁₆₅, VEGF₁₈₉ or VEGF₂₀₆. In a further embodiment, the activity of the VEGF is increased. In another embodiment, the activity of a FGF is also modulated. In yet another embodiment, the activity of the FGF is increased.

In still another embodiment, the composition is administered to a subject. In a further embodiment, the subject has a wound, scar, chronic liver disease, benign hyperplastic hypertrophy, cancer or an inflammatory disease. In yet a further embodiment, the subject has a disease associated with excessive VEGF-mediated angiogenesis. In still a further embodiment, the disease associated with excessive VEGF-mediated angiogenesis is age-related macular degeneration (AMD) or diabetic neuropathy. In yet another embodiment, the subject is in need of angiogenesis inhibition. In still another embodiment, the composition further comprises a pharmaceutically acceptable carrier. In a further embodiment, the composition further comprises an additional therapeutic agent. In another embodiment, the additional therapeutic agent is an anti-cancer agent or an anti-inflammatory agent. In a further embodiment, the additional therapeutic agent is a FGF and/or VEGF. In yet another embodiment, the method further comprises determining the presence or absence of the VEGF in the subject. In still another embodiment, the method further comprises determining the presence or absence of a FGF in the subject. In a further embodiment, the FGF is FGF7. In yet a further embodiment, the determining step is performed prior to the contacting step.

In still another aspect of the invention, a method of treating a subject comprising administering to a subject in need of such treatment a composition comprising GAGs of a highly sulfated GAG fraction, wherein the GAGs of the highly sulfated GAG fraction are administered in an amount effective to modulate an activity of a VEGF. In one embodiment, the highly sulfated GAG fraction is a highly sulfated CS fraction or a highly sulfated DS fraction. In a further embodiment, the subject has a wound, scar, chronic liver disease, benign hyperplastic hypertrophy, cancer or an inflammatory disease. In yet a further embodiment, the subject has a disease associated with excessive VEGF-mediated angiogenesis. In still a further embodiment, the disease associated with excessive VEGF-mediated angiogenesis is age-related macular degeneration (AMD) or diabetic neuropathy. In yet another embodiment, the subject is in need of angiogenesis inhibition. In still another embodiment, the composition further comprises a pharmaceutically acceptable carrier. In a further embodiment, the composition further comprises an additional therapeutic agent. In another embodiment, the additional therapeutic agent is an anti-cancer agent or an anti-inflammatory agent. In a further embodiment, the additional therapeutic agent is a FGF and/or VEGF. In yet another embodiment, the method further comprises determining the presence or absence of the VEGF in the subject. In still another embodiment, the method further comprises determining the presence or absence of a FGF in the subject. In a further embodiment, the FGF is FGF7. In yet a further embodiment, the determining step is performed prior to the contacting step.

Aspects of the invention relate to methods of producing an oversulfated GAG. In one embodiment, the oversulfated GAG is an oversulfated DS or oversulfated CS. The method in one embodiment comprises obtaining a fragment of the DS or CS and sulfating the fragment. In one embodiment, the sulfating is carried out with chemical oversulfation, such as with triethylamine sulfur trioxide. In one embodiment, the fragment is a fragment containing 4-O or 6-O sulfated disaccharides. In another embodiment, the method also comprises the step of partially fractionating, digesting a glycosaminoglycan prior to obtaining the fragment. In yet a further embodiment, the glycosaminoglycan(s) obtained from the partial fractionation or partial digestion is sulfated. Partial digestion can be carried out with a glycosaminoglycan-degrading enzyme, such as a chondroitinase. In a further embodiment, these glycosaminoglycans are then degraded (e.g., enzymatically degraded, such as with a chondroitinase). The degraded glycosaminoglycans can then be isolated or further sulfated and isolated. In an embodiment, the fragment is a tetrasaccharide, hexasaccharide, octasaccharide or a decasaccharide. In another embodiment, the fragment has or has less than 30 saccharide units. In another embodiment, the fragment has or has less than 25 saccharide units. In a further embodiment, the fragment has or has less than 20 saccharide units. In another embodiment, the fragment has or has less than 18 saccharide units. In yet another embodiment, the fragment has or has less than 16 saccharide units. In a further embodiment, the fragment has or has less than 14 saccharide units. In yet a further embodiment, the fragment has or has less than 12 saccharide units. In another embodiment, the method further comprises analyzing the oversulfated fragment. In yet another embodiment, the analyzing comprises assessing an activity of the oversulfated fragment. In still another embodiment, the activity is the modulation of a FGF activity, VEGF activity or both. In yet another embodiment, the activity is thrombin inhibition by heparin cofactor 2.

In aspects of the invention, compositions are provided. The compositions include the oversulfated GAGs (e.g., oversulfated CS or DS) produced by any of the aforementioned methods. In an embodiment, compositions further include a pharmaceutically acceptable carrier. In another embodiment, compositions further include an additional therapeutic agent. In a further embodiment, the additional therapeutic agent is a FGF and/or VEGF.

In aspects of the invention, compositions are provided as are methods for their use. In some embodiments, the compositions include a highly sulfated DS wherein at least 20%, 25%, 30%, 40%, 50%, 60%, 70%, 80%, 85%, 90%, 95% or more of the disaccharides of the highly sulfated DS are ΔDi 2S,4S. In other embodiments, at least 20%, 25%, 30%, 40%, 50%, 60%, 70%, 80%, 85%, 90%, 95% or more of the disaccharides of the highly sulfated DS are ΔDi 4S,6S. In still another embodiment, the highly sulfated DS contains about 4-5% ΔDi 2S,4S,6S, about 4-5% ΔDi 2S,4S, about 40% ΔDi 4S,6S and about 50% ΔDi 4S. The compositions can also include a highly sulfated DS, where at least 40% of the disaccharides are ΔDi 4S,6S. In an embodiment, at least 4% of the disaccharides are ΔDi 2S,4S. In another embodiment, 5% of the disaccharides are ΔDi 2S,4S. In a further embodiment, at least 4% of the disaccharides are ΔDi 2S,4S,6S. In another embodiment, 5% of the disaccharides are ΔDi 2S,4S,6S. In a further embodiment, the compositions include a highly sulfated CS wherein at least 20%, 25%, 30%, 40%, 50%, 60%, 70%, 80%, 85%, 90%, 95% or more of the disaccharides of the highly sulfated CS are ΔDi 2S,6S. In other embodiments, at least 20%, 25%, 30%, 40%, 50%, 60%, 70%, 80%, 85%, 90%, 95% or more of the disaccharides of the highly sulfated CS are ΔDi 4S,6S. In still other embodiments, at least 65%, 70%, 75%, 80%, 85%, 90%, 95% or more of the disaccharides of the highly sulfated CS are ΔDi 4S,6S. In still a further embodiment, compositions further include a pharmaceutically acceptable carrier. In yet another embodiment, compositions further include an additional therapeutic agent. In other embodiments, the compositions can be administered to a subject in need of anti-coagulation. In a further embodiment, the additional therapeutic agent is a FGF and/or VEGF.

Aspects of the invention relate to methods of modulating an activity of a FGF, comprising contacting the FGF with any of the aforementioned compositions. In an embodiment, the contacting is carried out by administering the composition to a subject.

Aspects of the invention relate to methods of modulating an activity of a VEGF, comprising contacting the VEGF with any of the aforementioned compositions.

Aspects of the invention relate to methods of modulating an activity of a FGF and an activity of a VEGF, comprising contacting the FGF and VEGF with any of the aforementioned compositions. In an embodiment, the contacting is carried out by administering the composition to a subject.

In a further aspect of the invention, the aforementioned compositions are used in the various methods of treating a subject as provided herein.

In another aspect of the invention, uses of the compositions provided for the preparation of a medicament are also provided.

For the methods provided herein, when “GAG” alone is recited it is intended that the method can also be one in which a composition comprising the GAG is used.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 illustrates that GAGs differentially promote FGF7-mediated effects. NBT-II cells were treated with FGF7 supplemented with GAGs. The inhibitory effect was measured by reduction in whole cell number relative to untreated cells (FIG. 1A). Cells were treated with sodium chlorate (FIG. 1B). The proliferative effect was measured by increase in whole cell number compared to cells treated with sodium chlorate only.

FIG. 2 illustrates that GAGs modulate FGFs and VEGFs. RT-PCR of NBT-II cells for Act (A), FGFR isoforms 1b, 1c, 2b, 2c, 3b, 3c and 4, and VEGR isoforms 1, 2 and 3 (FIG. 2A). NBT-II cells were treated with 10 ng/ml FGF1 or VEGF with varying concentrations of heparin (FIG. 2B). Data are presented as percent inhibition of cell growth compared to ligand alone. NBT-II cells were treated with 10 ng/ml FGF1 or VEGF with varying concentrations of UDS (FIG. 2C). Data are presented as percent inhibition of cell growth compared to ligand alone.

FIG. 3 shows that heparin and DS DT differentially impact the co-administration of FGF7 and VEGF. NBT-II cells were treated with 10 ng/ml of one of FGF1 or VEGF, as well as 10 ng/ml FGF7. Cells were additionally treated with heparin (FIG. 3A), UDS (FIG. 3B) or DS DT (FIG. 3C) over a range of concentrations. The effect of GAG addition was normalized to the effect of the ligand pair alone. The legend in FIG. 3A applies to FIGS. 3A-3C. Cells were treated with VEGF and FGF7 and supplemented with either heparin or DS DT (FIG. 3D). The proliferative effect was normalized to the effect of VEGF and FGF7 unsupplemented by GAGs.

FIG. 4 shows that VEGF induces proliferation through Erk and Mek. NBT-II cells were treated with FGF7, VEGF, or FGF7 and VEGF in the presence of PBS, heparin or DS DT. ELISAs were performed for phospho-Erk1/2 (FIG. 4A) and phospho-Mek1/2 (FIG. 4B). The change in response was determined in terms of its relative level compared to untreated cells. * denotes p<0.05 compared to untreated cells.

FIG. 5 illustrates that FGF7 affects proliferation through Akt. NBT-II cells were treated with FGF7, VEGF, or FGF7 and VEGF in the presence of PBS, heparin or DS DT. ELISAs were performed for phospho-Akt1/2/3 (Ser 473) (FIG. 5A) phospho-Akt1/2/3 (Thr 308) (FIG. 5B). The change in response was determined in terms of its relative level compared to untreated cells. * denotes p<0.05 compared to untreated cells.

FIG. 6 shows that FGF7 and VEGF upregulate VEGF-C and VEGF-D. NBT-II cells were treated with FGF7, VEGF, FGF7 and VEGF (FIGS. 6A-6C) or FGF2 in the presence of PBS, heparin or DS DT (FIG. 6D). ELISAs were performed after 24 hours for VEGF-C (FIG. 6A), VEGF-D (FIG. 6B), VEGFR3 (FIG. 6C) or both VEGF-C and VEGF-D (FIG. 6D). The change in response was determined in terms of its relative level compared to untreated cells. * denotes p<0.05 compared to untreated cells.

FIG. 7 illustrates that heparin and UDS differentially regulate VEGF-D. NBT-II cells were treated with VEGF, VEGF-C and VEGF-D either alone (FIG. 7A) or with FGF7 (FIG. 7B). Ligands were supplemented with heparin or DS DT. The proliferative effect was determined by whole cell count. Data was converted to the percent inhibition in total cell number compared to untreated cells.

FIG. 8 shows that chemical oversulfation of DS DT increases FGF7 activity. NBT-II cells were treated with 10 ng/ml FGF7 supplemented with PBS or GAGs at concentrations ranging between 1 and 100,000 ng/ml. The reduction in whole cell number observed in the presence of GAGs and FGF7 was normalized to that observed with FGF7 alone. * denotes p<0.05 for ddDS compared to DS DT at the same concentration. † denotes p<0.05 for diDS compared to DS DT at the same concentration. § denotes p<0.05 for CS D compared to DS DT at the same concentration. GAGs did not otherwise elicit a significantly different effect than DS DT at the same concentration.

FIG. 9 shows the structure of CS/DS.

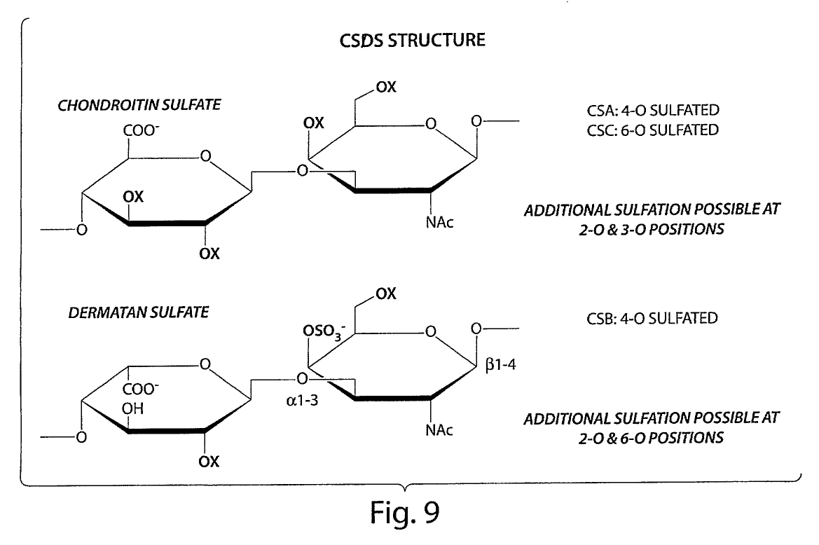

FIG. 10 provides results from a DS compositional analysis.

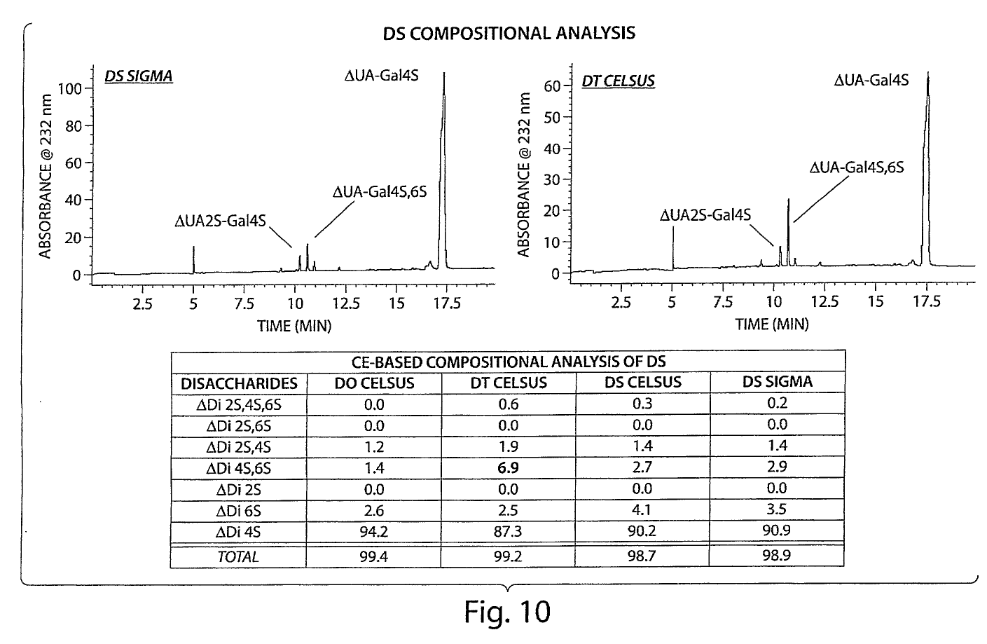

FIG. 11 provides results from a CE-compositional analysis.

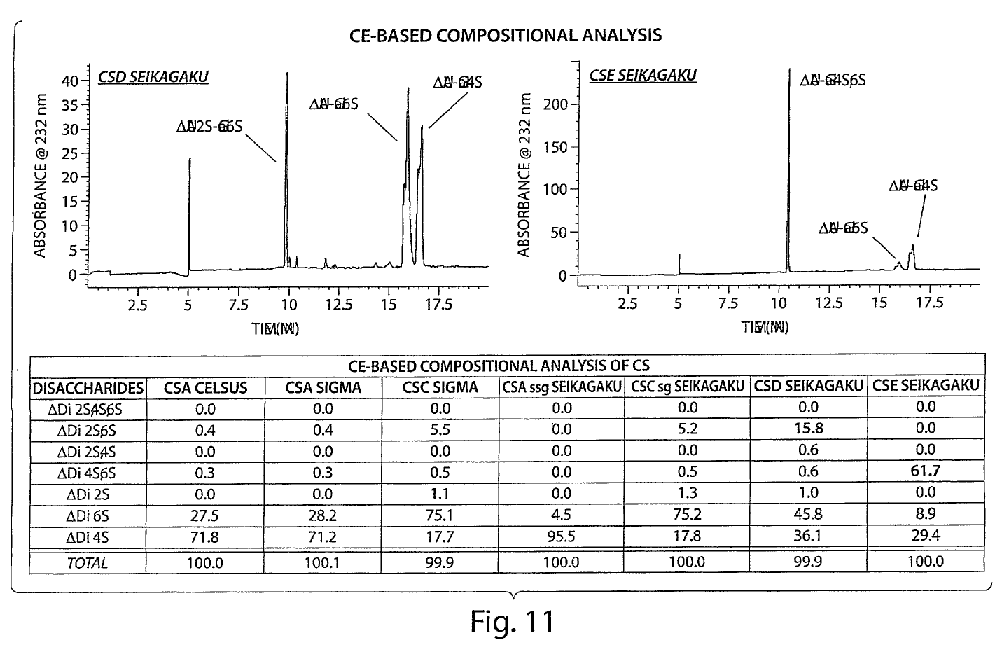

FIG. 12 provides results from the generation of defined CS/DS oligosaccharides.

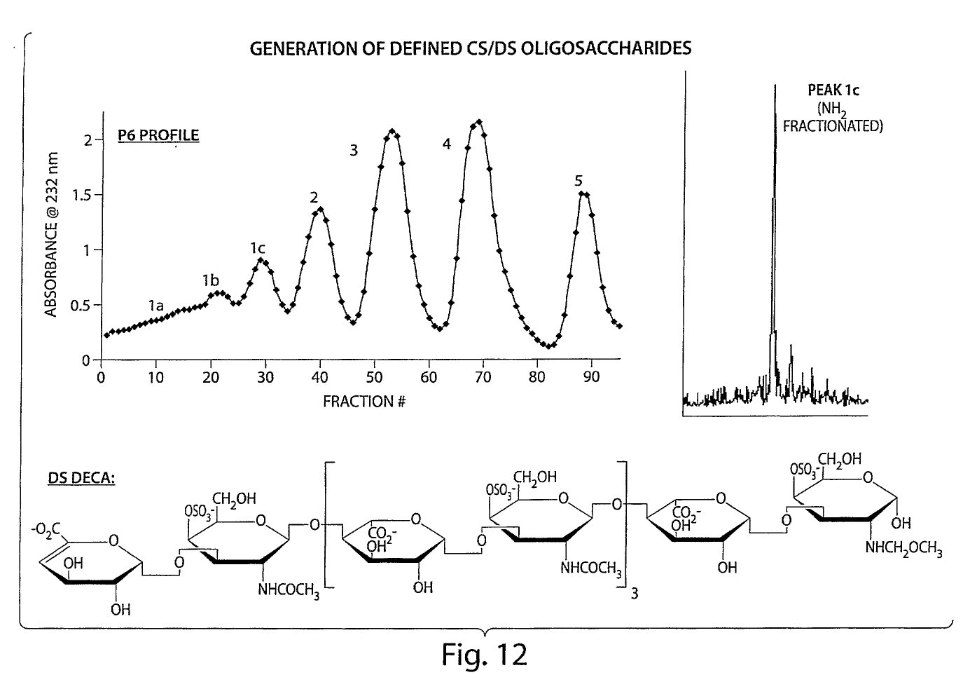

FIG. 13 provides results from a direct SAX purification of DT oligosaccharides.

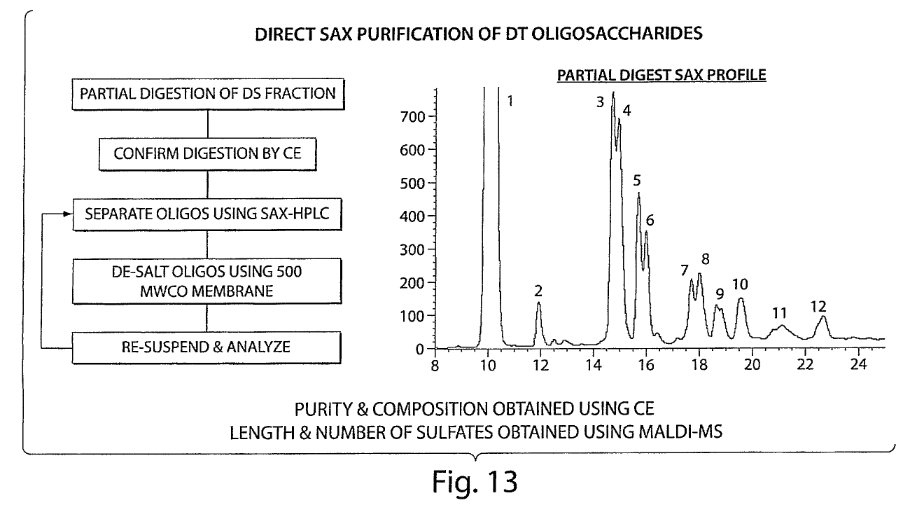

FIG. 14 provides a method for chemical sulfation as well as results from a compositional analysis.

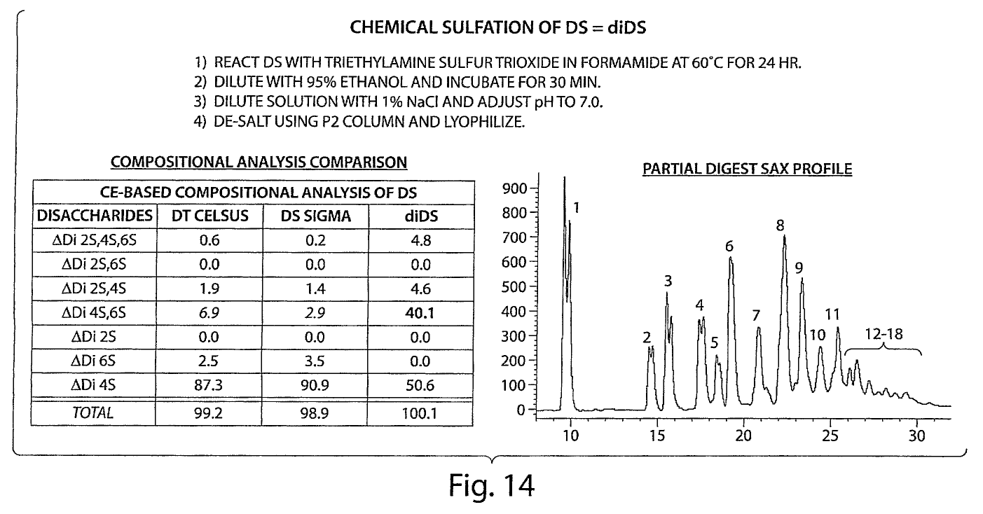

### DETAILED DESCRIPTION

GAGs are complex polysaccharides that exist both on the cell surface and free within the extracellular matrix. The intrinsic sequence variety stemming from the large number of building blocks that compose these biopolymers leads to substantial information density as well as to the ability to regulate a wide variety of important biological processes.

The capacity of various GAGs, including but not limited to HSGAGs, to regulate FGF and VEGF proteins in rat bladder cancer cells was investigated. Using FGF7 as a model growth factor, due to its specificity for a single FGFR isoform, how various GAGs altered its proliferative effects was examined. Heparin, the highly sulfated DS fraction DT (DS DT) and chondroitin sulfates, for example, were found to promote FGF7 activity. The analysis was also extended to FGF1, FGF2 and VEGF, and the activities of these growth factors were found, for example, to be affected, with similar magnitude and effect, by both heparin and DS DT. In addition, it was found that chemically oversulfated GAGs can increase FGF-mediated responses, such as FGF7-mediated response. Whether GAGs could regulate or even define the biological effect with multiple growth factors in the same cellular environment was also examined. It was found, for example, that heparin and highly sulfated DS differentially regulated the combination of FGF7 and VEGF. Heparin and DS DT, however, differentially regulated FGF7 and VEGF in the same cellular environment. This response stems primarily from the upregulation of VEGF-D, which itself, is differentially regulated by heparin and DS DT. VEGF-D-mediated cellular response occurs through VEGFR3.

All of the findings demonstrate that various GAGs can regulate the activities of FGF and VEGF proteins independently and/or in a common environment. Selectively developed GAGs, therefore, offer a way to select for the activity of growth factor subsets even in a complex pool, such as that which exists in healing wounds and in the tumor microenvironment. Provided herein, therefore, are compositions and methods for modulating the activity of a FGF and/or VEGF. As used herein, “modulating an activity of a FGF” refers to causing a change in an activity of a FGF in a sample or system (such as in a subject) that is present in the absence of a composition of the invention. This change can be an increase or decrease in the level or rate of an activity, the stimulation of an activity that is not otherwise present or the elimination of an activity altogether. In preferable embodiments, the modulating is causing an increase in an activity of the FGF. As used herein, an “increase” is the stimulation of an activity that is not present or is an increase in the level or rate of an activity. A decrease, as used herein, refers to the reduction in the level or rate of an activity or the complete elimination of an activity. Generally, the modulation can result when a composition provided herein is placed in contact with the FGF. The modulation can, for example, result when a composition of the invention is added to a sample containing a FGF. The modulation can also result when a composition provided herein is administered to a subject in which FGF is present. Likewise, “modulating an activity of a VEGF” refers to causing a change in an activity of a VEGF in a sample or system (such as in a subject) that is present in the absence of a composition of the invention. This change can be an increase or decrease in the level or rate of an activity, the stimulation of an activity that is not otherwise present or the elimination of an activity altogether. In preferable embodiments, the modulating is causing an increase in an activity of the VEGF. Generally, the modulation can result when a composition provided herein is placed in contact with the VEGF. The modulation can result when a composition of the invention is added to a sample containing a VEGF and can also result when a composition provided herein is administered to a subject in which VEGF is present. The compositions of the invention can, in some embodiments, modulate an activity of both an FGF and VEGF. The modulation can be an increase in the activity of both the FGF and VEGF, can be a decrease in an activity of both the FGF and VEGF or can be an increase in an activity of one and a decrease in an activity of the other. The modulating of a FGF and/or VEGF, as used herein, is intended to refer to the modulation an activity of the protein(s). The modulation of an activity of FGF and/or VEGF, in some embodiments, results in the promotion of cell proliferation and/or angiogenesis. In other embodiments, the modulation results in the inhibition of cell proliferation and/or angiogenesis.

As stated above, the compositions provided herein can modulate an activity of a FGF and/or VEGF when placed in contact with the FGF and/or VEGF. “Contacting” or “placing in contact” is meant to refer to causing a composition of the invention to be close in enough proximity to a FGF and/or VEGF such that it modulates an activity of the FGF and/or VEGF. In some embodiments, the composition binds to the FGF and/or VEGF. In other embodiments, the composition binds to a protein that causes downstream regulation of the FGF and/or VEGF. In still other embodiments, the composition is provided to a sample containing a FGF and/or VEGF. In yet other embodiment, the composition is administered to a subject in which FGF and/or VEGF is present. The composition can be administered in such a way so that the composition or a portion thereof modulates and activity of a FGF and/or VEGF. Such methods of administration will be apparent to those of ordinary skill in the art. Examples are also provided herein.

For instance, the composition may be administered by any of the routes of administration described herein such that the composition is delivered to the site of action. If the composition is delivered to a subject to treat a cancer, in some embodiments, it is desirable to deliver the composition to the site of the cancer, either directly or indirectly or to deliver it to the site of unwanted angiogenesis. Directly delivering a composition to a site of a cancer may involve direct injection or implantation at the site. Indirect delivery may involve systemic delivery such that the body delivers the active component to the site of action. The site of action is, in some embodiments, the site where FGF and/or VEGF are functioning. Alternatively, the composition may be delivered in conjunction with FGF and/or VEGF. As used herein “in conjunction” refers to delivery to the same subject in the same vehicle or separate vehicles, at the same or different times, at the same or different sites and by the same or different routes of administration. The co-delivered FGF and/or VEGF may be a nucleic acid that expresses functional FGF and/or VEGF or it may be a peptide.

In some embodiments, the methods provided also comprise the step of determining the presence or absence of one or more FGFs and/or one or more VEGFs. In other embodiments, the presence or absence of at least one FGF and at least one VEGF is determined. In still another embodiment the FGF is FGF1, FGF2, FGF7, FGF10 or FGF20. In a further embodiment, the amount of at least one FGF and/or at least one VEGF is determined. It will be readily apparent to one of ordinary skill in the art that there are a number of ways to determine the presence or absence or amount of a protein or RNA in a sample. For example, the presence or absence or amount of a protein in a sample can be assessed using antibodies to the protein. Preferably, the antibodies are detectably labeled. The label can be, for example, a fluorescent label, an enzyme label, a radioactive label, a luminescent label or a chromophore label. The amount of protein can also be determined, for instance, using northern or western blot analysis or other binding assays or any other method known to those of skill in the art. Detection of RNA can be carried out using nucleic acid probes or primers, such as with PCR, to bind to RNA (e.g., mRNA) in the sample. The sample in some embodiments, can be a sample from a subject (e.g., a blood, urine or tissue sample).

One of ordinary skill in the art is able to recognize the proteins that are FGFs or VEGFs. As mentioned above, the FGF family consists of at least 23 members. All the members of the FGF family bind glycosaminoglycans, such as heparin, and retain structural homology across species, suggesting a conservation of their structure/function relationship (Ornitz et al., J. Biol. Chem. 271(25):15292-15297, 1996.). A protein is a member of the FGF family, as used herein, if it shows significant sequence and three-dimensional structural homology to other members of the FGF family, FGF-like activity in in vitro or in vivo assays and binds to glycosaminoglycan or glycosaminoglycan-like substances and/or has an activity that can be regulated with glycosaminoglycans and/or glycosaminoglycan-like substances. FGFs include, but are not limited to FGF1, FGF2, FGF7, FGF10 and FGF20. FGF7, for example, is characterized as having an important role in inflammatory bowel disease and pulmonary epithelial injury. FGF7 overactivity has also been associated with colorectal cancer and benign prostatic hypertrophy (BPH). Also as mentioned above, VEGFs can regulate cell growth and angiogenesis. There are a number of VEGF isoforms, and they show variable interactions with GAGs. As used herein, a protein is a member of the VEGF family if it shows significant sequence and/or three-dimensional structure homology to other members of the VEGF family, have VEGF-like activity in in vitro or in vivo assays and can bind to glycosaminoglycans and/or glycosaminoglycan-like substances and/or has an activity that can be regulated with glycosaminoglycans and/or glycosaminoglycan-like substances. VEGFs, therefore, include, for example, VEGF-A, VEGF-C and VEGF-D. VEGFs also include isoforms and splice variants of the foregoing. VEGFs, therefore, also include mouse VEGF-A isoforms (VEGF₁₂₀, VEGF₁₆₄ and VEGF₁₈₈) and human VEGF-A isoforms (VEGF₁₂₁, VEGF₁₄₅, VEGF₁₆₅, VEGF₁₈₉ and VEGF₂₀₆ isoforms).

The activity of a FGF and/or VEGF can be modulated with a GAG or GAG fraction. Compositions of and methods for modulating an activity of a FGF and/or VEGF with a GAG or GAG fraction are, therefore, provided. Members of the GAG family of complex polysaccharides include DS, CS, HSGAG, keratan sulfate and hyaluronic acid. CS and DS glycosaminoglycan polysaccharides, have been implicated in biological processes ranging from osteoarthritis to anticoagulation. DS is a member of a subset of the GAG family referred to as galactosaminoglycans (GalAGs). Galactosaminoglycans are composed of a disaccharide repeat unit of uronic acid [-L-iduronic (IdoA) or -D-glucuronic (GlcA)] linked to N-acetyl-D-galactosamine (GalNAc). These basic disaccharide units are linearly associated to form polymers of chondroitin sulfate (CS) or dermatan sulfate (DS). The uronic acids of CS are exclusively GlcA; with DS, epimerization at the C-5 position of the uronic acid moiety during biosynthesis results in a mixture of IdoA and GlcA epimers. CS can be O-sulfated at the C-4 of the galactosamine(chondroitin-4-sulfate, C4S or CS A) or the C6 of the galactosamine(chondroitin-6-sulfate, C6S or CS C). For DS, C-4 sulfation of the galactosamine is a common modification and O-sulfation at C-2 of the IdoA moiety may also occur. Other rare modifications in CS, such as 2-O or 3-O sulfation of the GicA moiety, have also been reported (Nadanaka, S, and Sugahara, K. (1997) Glycobiology 7, 253-263; Sugahara, K., et al. (1996) J Biol Chem 271, 26745-26754.) The GAG family, therefore, includes chondroitin and dermatan sulfate GAGs, such as C4S, C6S, DS, chondroitin, chondroitin D, chondroitin E, chondroitin sulfate D (CS D), chondroitin sulfate E (CS E) and hyaluronan.

An activity of a FGF (e.g., FGF7) and/or a VEGF (e.g., VEGF-D) can be modulated with highly sulfated glycosaminoglycans or undersulfated glycosaminoglycans. The GAGs for use in the compositions and methods provided, therefore, can be highly sulfated GAGs. “Highly sulfated GAGs” are intended to be glycosaminoglycans or glycosaminoglycan fragments in which the majority of the disaccharides of the glycosaminoglyan are di- or tri-sulfated. Highly sulfated glycosaminoglycans, therefore, include glycosaminoglycans or glycosaminoglycan fragments thereof, wherein at least 20%, 25%, 30%, 40%, 50%, 60%, 70%, 80%, 85%, 90%, 95% or more of the disaccharides are di-sulfated. Highly sulfated GAGs also include glycosaminoglycans or glycosaminoglycan fragments thereof, wherein at least 20%, 25%, 30%, 40%, 50%, 60%, 70%, 80%, 85%, 90%, 95% or more of the disaccharides are tri-sulfated. Highly sulfated GAGs further include glycosaminoglycans or glycosaminoglycan fragments thereof, wherein at least 20%, 25%, 30%, 40%, 50%, 60%, 70%, 80%, 85%, 90%, 95% or more of the disaccharides are either di- or tri-sulfated.

Highly sulfated glycosaminoglycans also includes highly sulfated dermatan sulfates and chondroitin sulfates. Highly sulfated dermatan sulfates are dermatan sulfates wherein at least 20%, 25%, 30%, 40%, 50%, 60%, 70%, 80%, 85%, 90%, 95% or more of the disaccharides are di-sulfated, tri-sulfated or either di-sulfated or tri-sulfated. Likewise, highly sulfated chondroitin sulfates are chondroitin sulfates wherein at least 20%, 25%, 30%, 40%, 50%, 60%, 70%, 80%, 85%, 90%, 95% or more of the disaccharides are di-sulfated, tri-sulfated or either di-sulfated or tri-sulfated. In some embodiments, at least 40%, 50%, 60%, 70% or 50% of the highly sulfated dermatan sulfate or highly sulfated chondroitin sulfate are either di- or tri-sulfated. In some embodiments, at least 20%, 25%, 30%, 40%, 50%, 60%, 70%, 80%, 85%, 90%, 95% or more of the disaccharides of the highly sulfated DS are ΔDi 2S,4S. In other embodiments, at least 20%, 25%, 30%, 40%, 50%, 60%, 70%, 80%, 85%, 90%, 95% or more of the disaccharides of the highly sulfated DS are ΔDi 4S,6S. In one embodiment, the highly sulfated DS contains about 4-5% ΔDi 2S,4S,6S, about 4-5% ΔDi 2S,4S, about 40% ΔDi 4S,6S and about 50% ΔDi 4S. Compositions comprising this DS are also provided as are methods of their use. In some embodiments, at least 20%, 25%, 30%, 40%, 50%, 60%, 70%, 80%, 85%, 90%, 95% or more of the disaccharides of the highly sulfated CS are ΔDi 2S,6S. In other embodiments, at least 20%, 25%, 30%, 40%, 50%, 60%, 70%, 80%, 85%, 90%, 95% or more of the disaccharides of the highly sulfated CS are ΔDi 4S,6S. In still other embodiments, at least 65%, 70%, 75%, 80%, 85%, 90%, 95% or more of the disaccharides of the highly sulfated CS are ΔDi 4S,6S.

The highly sulfated glycosaminoglycans can be obtained from nature or can be produced to have certain levels of sulfation. The process of altering a naturally occurring glycosaminoglycan to have certain levels of sulfation is also referred to herein as “oversulfation” or “undersulfation”. Oversulfation refers to increasing the amount of sulfation of a naturally occurring glycosaminoglycan. Undersulfation refers to decreasing the amount of sulfation of a naturally occurring glycosaminoglycan. Compositions of and methods of using oversulfated and undersulfated glycosaminoglycans are also provided herein. Oversulfated glycosaminoglycans are intended to be included in the use of the term highly sulfated glycosaminoglycans.

Oversulfated glycosaminoglycans include oversulfated DS and oversulfated CS. Commercial DS is predominantly sulfated at the 4-O position of the N-acetyl galactosamine (GalNac) residue. In addition, small amounts of 2-O/4-O and 4-O/6-O disulfated disaccharides are present. It is possible, for example, to specifically increase the sulfation of commercial DS at the 6-O position of the GalNac moiety. Similar to DS, commercially available chondroitin sulfate A is characterized by primary sulfation at the 4-O position of GalNac. CSA can also be chemically sulfated in an attempt to generate 4-O/6-O disulfated chondroitin sulfate (CSD). The production of oversulfated dermatan sulfates and oversulfated chondroitin sulfates has been previously described (U.S. Pat. Nos. 5,382,570, 5,529,985, 5,668,274; 5,922,690; 6,486,137) and are provided herein. Briefly, a glycosaminoglycan or fragment thereof can be reacted with triethylamine sulfur trioxide in formamide at 60° C. for 24 hours. The sample can then be diluted with 95% ethanol and incubated for 30 minutes. The sample can then be diluted with 1% NaCl and the pH adjusted to 7.0. The sample can then be desalted using P2 column and lyophilized. Preferably, the reaction increases the percentage of 4-O/6-O disulfated disaccharides in the polymer from 3% to 40%. The method can also include first partially digesting the glycosaminoglycan, such as with a glycosaminoglycan-degrading enzyme, such as chondroitinase B. A “glycosaminoglycan-degrading enzyme” is any enzyme that somehow modifies a glycosaminoglycan. The modification can be cleavage. Such enzymes include heparinases, chondroitinases (e.g., chondroitinase B, chondroitinase ABC, chondroitinase AC, etc.), glycuronidases, glucuronidases, sulfatases, etc. The method can also include a step of isolating the partially digested glycosaminoglycan fragments, such as specific 4-O or 6-O sulfated oligosaccharides, and it is these fragments that are subsequently chemically sulfated. Alternatively, such fragments can be obtained from a glycosaminoglycan that has been chemically sulfated, and the fragments are subjected to further sulfation. Methods of forming undersulfated dermatan sulfates and chondroitin sulfates have also been described (U.S. Patent Application No. 20030149253).

The oversulfated or undersulfated glycosaminoglycans that are produced can then be analyzed. The analysis, for example, can be any assessment of the effect of the oversulfated or undersulfated glycosaminoglycan on an activity of, for example, a FGF and/or VEGF. Examples of methods of such analysis are provided herein in the Examples. For example, activity can be assessed using the FGF/VEGF cell system described herein. As another example, an activity of the produced glycosaminoglycans can be assessed and compared with other glycosaminoglycans. The activity can be, for example, the ability to inhibit thrombin via the heparin cofactor II pathway. Other activities and assays are known in the art.

Glycosaminoglycan fractions can also be used to modulate a FGF and/or VEGF, either alone, or in a mixture of multiple growth factors (including multiple families). Therefore, in some embodiments, the GAGs for the compositions and methods provided are the GAGs of a highly sulfated GAG fraction. As used herein, a “highly sulfated GAG fraction” is a sample of GAGs in which the majority of the GAGs of the sample are highly sulfated. In some embodiments, at least 50%, 60%, 70%, 80%, 85%, 90%, 95% or more of the GAGs are highly sulfated. Generally, but not necessarily, such a sample is a fractionated portion of a larger sample of GAGs. Fractionation methods for selecting fractions of GAGs are well known in the art. In one embodiment, the highly sulfated GAG fraction is DS DT.

As used herein, in some embodiments, the GAGs or GAG fractions are substantially pure. The term “substantially pure” means that the GAGs or GAG fractions are essentially free of other substances to an extent practical and appropriate for their intended use. In some embodiments, a substantially pure compositions can be one that also contains one or more salts. In other embodiments, a substantially pure composition is one that does not contain one or more salts. In certain embodiments, the GAGs or GAG fractions are sufficiently pure and are sufficiently free from other biological constituents of the cells from which they are derived so as to be useful in, for example, pharmaceutical preparations. GAGs or GAG fractions can be isolated from biological samples, and can also be produced synthetically. In some embodiments, the compositions containing one or more GAGs or one or more GAG fractions is greater than 90% free of contaminants. Preferably, the material is greater than 91%, 92%, 93%, 94%, 95%, 96%, 97%, 98%, or even greater then 99% free of contaminants. In some embodiments, the contaminant can be a salt. In other embodiments, depending on the intended use, a salt is not considered a contaminant. The degree of purity may be assessed by means known in the art.

As used herein, a GAG may be isolated. “Isolated” means separated from its native environment and present in sufficient quantity to permit its identification or use. Isolated GAGs may be, but need not be, substantially pure. Because an isolated GAG may be admixed with a pharmaceutically acceptable carrier in a pharmaceutical preparation, the GAG may comprise only a small percentage by weight of the preparation. The GAG is nonetheless isolated in that it has been separated from the substances with which it may be associated in living systems.

In addition to GAGs from natural sources, the GAGs of the invention also include molecules that are biotechnologically prepared, chemically modified and synthetic. The term “biotechnological prepared” encompasses GAGs that are prepared from natural sources of GAGs which have been chemically modified. Such GAGs are known to those of skill in the art. Synthetic GAGs are also well known to those of skill in the art. As used herein a “sample” of GAGs is meant to include any sample which has one or more GAGs contained therein.

Also provided are a wide range of uses for the compositions provided herein. For example, enhancing FGF7 function by oversulfated DS, oversulfated CS and/or highly sulfated HSGAGs is useful in promoting wound healing, scar reduction, treating cancer (e.g., bladder cancer, etc.), treating inflammatory disease (e.g., inflammatory bowel disease) and promoting epithelial cell survival (e.g., pulmonary epithelial cell survival, such as after inhalation burns, etc.). As another example, enhancing VEGF-D function by oversulfated DS, oversulfated CS, and/or highly sulfated HSGAGs is useful in promoting microvessel enlargement (e.g., for tissue engineering, scar reduction, wound healing, etc.) and growing muscle. As a further example, inhibition of FGF7 activity in a mixture of growth factors, such as with heparin, has therapeutic value for treating, for example, chronic liver disease, excessive wound healing, cancer (e.g., colon/colorectal cancer, prostate cancer, pancreatic cancer) and BPH. Inhibition of VEGF-D activity in a mixture of growth factors, such as with heparin, has therapeutic value for treating cancer (e.g., prostate cancer and gastric cancer (primarily by preventing metastases)). As a further example, DS or highly or oversulfated DS, could be used to prevent diabetic nephropathy. DS, for example, supports the activities of FGF2 and FGF7. The compositions provided can also enhance heparin cofactor II-mediated inhibition of thrombin. Methods are, therefore, provided for treating a subject with any of the conditions, diseases or disorders described herein using a composition of the invention. Methods are also provided for enhancing or inhibiting a function described herein with a composition of the invention.

In some embodiments the inflammatory disease is non-autoimmune inflammatory bowel disease, post-surgical adhesions, coronary artery disease, hepatic fibrosis, acute respiratory distress syndrome, acute inflammatory pancreatitis, endoscopic retrograde cholangiopancreatography-induced pancreatitis, burns, atherogenesis of coronary, cerebral and peripheral arteries, appendicitis, cholecystitis, diverticulitis, visceral fibrotic disorders, wound healing, skin scarring disorders (keloids, hidradenitis suppurativa), granulomatous disorders (sarcoidosis, primary biliary cirrhosis), asthma, pyoderma gandrenosum, Sweet's syndrome, Behcet's disease, primary sclerosing cholangitis or an abscess. In some preferred embodiment the inflammatory disease is inflammatory bowel disease (e.g., Crohn's disease or ulcerative colitis).

The inflammatory disease can be an autoimmune disease. The autoimmune disease in some embodiments is rheumatoid arthritis, rheumatic fever, ulcerative colitis, Crohn's disease, autoimmune inflammatory bowel disease, insulin-dependent diabetes mellitus, diabetes mellitus, juvenile diabetes, spontaneous autoimmune diabetes, gastritis, autoimmune atrophic gastritis, autoimmune hepatitis, thyroiditis, Hashimoto's thyroiditis, insulitis, oophoritis, orchitis, uveitis, phacogenic uveitis, multiple sclerosis, myasthenia gravis, primary myxoedema, thyrotoxicosis, pernicious anemia, autoimmune haemolytic anemia, Addison's disease, Anklosing spondylitis, sarcoidosis, scleroderma, Goodpasture's syndrome, Guillain-Barre syndrome, Graves' disease, glomerulonephritis, psoriasis, pemphigus vulgaris, pemphigoid, sympathetic opthalmia, idiopathic thrombocylopenic purpura, idiopathic feucopenia, Siogren's syndrome, systemic sclerosis, Wegener's granulomatosis, poly/dermatomyositis, lupus or systemic lupus erythematosus.

The subject can be in need of wound healing or scar reduction. As used herein, a subject that is “in need of wound healing or scar reduction” is a subject with a wound or a scar in which the therapeutics provided herein would have some benefit. As used herein, the term “wound” is used to describe skin wounds and tissue wounds. A skin wound is defined herein as a break in the continuity of skin tissue which is caused by injury to the skin. Skin wounds are generally characterized by several classes including punctures, incisions, including those product by surgical procedures, excisions, lacerations, abrasions, atrophic skin, or necrotic wounds and burns. A “tissue wound” as used herein is a wound to an internal organ, such as a blood vessel, intestine, colon, etc. For instance, during the repair of arteries the vessel needs to be sealed and wound healing must be promoted.

The methods of the invention are also useful for preventing scar formation. The compositions can be use to prevent the formation of a scar at the same time as promoting wound healing. Alternatively, the compositions may be used for preventing scar formation by reducing or initiating regression of existing scars. Scar tissue as used herein refers to the fiber rich formations arising from the union of opposing surfaces of a wound. The term “reduction in scar formation” as used herein refers to the production of a scar smaller in size than would ordinarily have occurred in the absence of the active components and/or a reduction in the size of an existing scar.

The compositions of the invention are also useful for treating and preventing cancer cell proliferation and metastasis. Thus, according to another aspect of the invention, the subject is one that has or is at risk of having cancer. A “subject that has cancer” is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. Cancers also include cancer of the blood and larynx. A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission.

Additionally, the subject can also be one in which unwanted angiogenesis is occurring. Angiogenesis as used herein is the inappropriate formation of new blood vessels. “Angiogenesis” often occurs in tumors when endothelial cells secrete a group of growth factors that are mitogenic for endothelium causing the elongation and proliferation of endothelial cells which results in a generation of new blood vessels. The inhibition of angiogenesis can cause tumor regression in animal models, suggesting a use as a therapeutic anticancer agent. An effective amount for inhibiting angiogenesis is an amount which is sufficient to diminish the number of blood vessels growing into a tumor. This amount can be assessed in an animal model of tumors and angiogenesis, many of which are known in the art.

The subject can be one who has a disease associated with excessive VEGF-mediated angiogenesis. Such disease include, for example, age-related macular degeneration and diabetic neuropathy.

The subject can also be one in which the subject has chronic liver disease or BPH.

The terms “treat” and “treating”, as used herein, refer to inhibiting completely or partially a biological effect of a condition, disease or disorder, as well as inhibiting any increase in a biological effect of a condition, disease or disorder. When used in terms of treating an inflammatory disease, the terms are also intended to refer to inhibiting completely or partially an inflammatory response and/or resulting inflammation and/or a symptom of the inflammatory disease. When used in terms of treating cancer, the terms are intended to refer to inhibiting or eliminating cancer cell growth and/or a reduction or elimination of a symptom or side effect of the cancer. When used to refer to treating tumor cell proliferation, as used herein, the terms also refer to inhibiting completely or partially the proliferation or metastasis of a cancer or tumor cell, as well as inhibiting any increase in the proliferation or metastasis of a cancer or tumor cell.

Each of the conditions, diseases or disorders recited herein is well-known in the art and/or is described, for instance, in Harrison's Principles of Internal Medicine (McGraw Hill, Inc., New York), which is incorporated by reference.

The compositions provided can include an additional therapeutic agent. Similarly, the methods provided can also include contacting or administering an additional therapeutic agent. An “additional therapeutic agent” is any agent that can result is some benefit for any condition, disease or disorder that can be treated with the compositions of the invention and that is in addition to the compositions of the invention. In one embodiment, the additional therapeutic agent is a FGF or a VEGF. Therefore, compositions of the GAGs provided herein and a FGF or a VEGF or both are also provided. Methods of using such compositions as provided herein are also provided.

The additional therapeutic agent can be an anti-cancer agent. Anti-cancer agents include Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin and Zorubicin Hydrochloride.

Anti-cancer agents also can include cytotoxic agents and agents that act on tumor neovasculature. Cytotoxic agents include cytotoxic radionuclides, chemical toxins and protein toxins. The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Anti-cancer agents also include suitable chemical toxins or chemotherapeutic agents, such as members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Toxins also include poisonous lectins, plant toxins such as ricin, abrin, modeccin, botulina and diphtheria toxins. Of course, combinations of the various toxins are also provided thereby accommodating variable cytotoxicity. Other chemotherapeutic agents are known to those skilled in the art.

Anticancer agents also include immunomodulators such as α-interferon, β-interferon, and tumor necrosis factor alpha (TNF).

Additional therapeutic agents can be agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein), interferon inducible protein 10 (U.S. Pat. No. 5,994,292), and the like.

The additional therapeutic agent can be an anti-inflammatory agent. Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids and Zomepirac Sodium.

The compositions may be delivered with agents for the treatment of wounds such as, for instance, dexpanthenol, growth factors, enzymes or hormones, povidon-iodide, fatty acids, such as cetylphridinium chloride, antibiotics, and analgesics. Growth factors useful in would healing include, but are not limited to, fibroblast growth factor (FGF), FGF-1, FGF-2, FGF-4, platelet-derived growth factor (PDGF), insulin-binding growth factor (IGF), IGF-1, IGF-2, epidermal growth factor (EGF), transforming growth factor (TGF), TGF-α, TGF-β, cartilage inducing factors-A and -B, osteoid-inducing factors, osteogenin and other bone growth factors, collagen growth factors, heparin-binding growth factor-1 or -2, and/or their biologically active derivatives. The compositions may also include antiseptics.

As mentioned above, the compositions may also be delivered with FGF and/or VEGF. The FGF or VEGF may be a nucleic acid that expresses functional FGF or VEGF or it may be a peptide. The isolated FGF or VEGF nucleic acids of the invention also include nucleic acids encoding fragments of an intact FGF or VEGF. Preferably, the fragments are functional equivalents of the intact FGF or VEGF nucleic acid. For example, the FGF or VEGF nucleic acids may encode a fragment that is a “soluble FGF or VEGF polypeptide” or a fragment that is a “membrane-associated FGF or VEGF polypeptide”. Soluble FGF or VEGF polypeptides, nucleic acids encoding same, and vectors containing said nucleic acids are described. FGF nucleic acid sequences have been described in U.S. Pat. Nos. such as 6,844,193, 6,844,168, 6,797,695, 6,716,626, 6,518,236, and 6,403,557. VEGF nucleic acid sequences have been described in U.S. Pat. Nos. such as 7,005,505, 6,818,220, 6,783,954, 6,783,953 6,750,044 and 6,734,285 and in Genbank numbers NM_{—}001033756, NM_{—}001025370, NM_{—}001025369, NM_{—}001025368, NM_{—}001025367, NM_{—}003376, NM_{—}001025366.

The invention also embraces nucleic acid molecules that differ from the foregoing in that the nucleic acids encode a FGF or VEGF polypeptide that has one or more amino acid substitutions that don't knock out functionality.

The FGF and VEGF nucleic acids are known, as described above, but variants and other modified forms can be identified by conventional techniques, e.g., by identifying nucleic acid sequences which code for FGF or VEGF polypeptides and which hybridize to a nucleic acid molecule having the known sequences of FGF or VEGF under stringent conditions. The term “stringent conditions”, as used herein, refers to parameters with which the art is familiar. More specifically, stringent conditions, as used herein, refer to hybridization at 65° C. in hybridization buffer (3.5×SSC, 0.02% Ficoll, 0.02% polyvinyl pyrolidone, 0.02% bovine serum albumin, 2.5 mM NaH2PO4 (pH 7), 0.5% SDS, 2 mM EDTA). SSC is 0.15M sodium chloride/0.15M sodium citrate, pH 7; SDS is sodium dodecyl sulphate; and EDTA is ethylenediaminetetraacetic acid. After hybridization, the membrane to which the DNA is transferred is washed at 2×SSC at room temperature and then at 0.1×SSC/0.1×SDS at 65° C.

There are other conditions, reagents, and so forth which can be used, which result in a similar degree of stringency. The skilled artisan will be familiar with such conditions and, thus, they are not given here. It will be understood, however, that the skilled artisan will be able to manipulate the conditions in a manner to permit the clear identification of homologs and alleles of FGF or VEGF nucleic acids. The skilled artisan also is familiar with the methodology for screening cells and libraries for the expression of molecules, such as FGF or VEGF, can be isolated, following by isolation of the pertinent nucleic acid molecule and sequencing. In screening for FGF or VEGF nucleic acid sequences, a Southern blot may be performed using the foregoing conditions, together with a radioactive probe. After washing the membrane to which the DNA is finally transferred, the membrane can be placed against x-ray film to detect the radioactive signal.

In general, homologs and alleles typically will share at least 40% nucleotide identity with known functional FGF or VEGF nucleic acids; in some instances, will share at least 50% nucleotide identity; and in still other instances, will share at least 60% nucleotide identity. Watson-Crick complements of the foregoing nucleic acids are also useful. The homologs may have at least 70%, 80% or 90% sequence homology.

Useful nucleic acids also include degenerate nucleic acids which include alternative codons to those present in the naturally occurring nucleic acids that code for the human FGF or VEGF polypeptide. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide codons may be employed to direct the protein synthesis apparatus, in vitro or in vivo, to incorporate a serine residue. Similarly, nucleotide sequence triplets which encode other amino acid residues include, but are not limited to, CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences.

The FGF or VEGF nucleic acid, in one embodiment, is operably linked to a gene expression sequence which directs the expression of the FGF or VEGF nucleic acid within a eukaryotic cell. The “gene expression sequence” is any regulatory nucleotide sequence, such as a promoter sequence or promoter-enhancer combination, which facilitates the efficient transcription and translation of the FGP or VEGF nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, β-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

As used herein, a “FGF or VEGF peptide or polypeptide” refers to a functional FGF or VEGF. FGF or VEGF polypeptides further embrace functionally equivalent variants, and analogs of known FGF or VEGF peptides, provided that the fragments, variants, and analogs are functional. Accordingly, it is intended that polypeptides which have the amino acid sequence of FGF or VEGF but which include conservative substitutions are embraced within the instant invention. As used herein, “conservative amino acid substitution” refers to an amino acid substitution which does not alter the relative charge or size characteristics of the polypeptide in which the amino acid substitution is made. Conservative substitutions of amino acids include substitutions made amongst amino acids with the following groups: (1) M,I,L,V; (2) F,Y,W; (3) K,R,H; (4) A,G; (5) S,T; (6) Q,N; and, (7) E,D.

Effective amounts of the compositions of the invention are administered to subjects in need of such treatment. Effective amounts are those amounts which will result in a desired improvement in the condition, disease or disorder or symptoms of the condition, disease or disorder. Effective amounts also include those amount that lead to the desired endpoint. Such amounts can be determined with no more than routine experimentation. As used herein, an amount “effective to modulate a FGF or VEGF activity” is any amount of the agents of the invention alone or in combination with an additional therapeutic agent that is effective to modulate an activity of the FGF and/or VEGF. The modulation can be an increase or decrease in activity.

It is believed that doses ranging from 1 nanogram/kilogram to 100 milligrams/kilogram, depending upon the mode of administration, will be effective. In some embodiments the level of administration is between 3 micrograms to 14 milligrams per 4 square centimeter area of cells. The absolute amount will depend upon a variety of factors (including whether the administration is in conjunction with other methods of treatment, the number of doses and individual patient parameters including age, physical condition, size and weight) and can be determined with routine experimentation. It is preferred, generally, that a maximum dose be used, that is, the highest safe dose according to sound medical judgment. The mode of administration may be any medically acceptable mode including oral, ocular, topical, transdermal, rectal, nasal, subcutaneous, intravenous, etc. or via administration to a mucous membrane. In some embodiments the mode of administration is topical administration.

In general, when administered for therapeutic purposes, the formulations of the invention are applied in pharmaceutically acceptable solutions. Such preparations may routinely contain pharmaceutically acceptable concentrations of salt, buffering agents, preservatives, compatible carriers, adjuvants and optionally other therapeutic ingredients.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The present invention provides pharmaceutical compositions, for medical use, which comprise the one or more agents of the invention together with one or more pharmaceutically acceptable carriers and optionally other therapeutic ingredients. The pharmaceutical compositions can also, in some embodiments, include one or more additional therapeutic agents. The term “pharmaceutically-acceptable carrier” as used herein, and described more fully below, means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being commingled with each other, in a manner such that there is no interaction which would substantially impair the desired pharmaceutical efficiency. The pharmaceutically acceptable carrier can, in some embodiments, be sterile.

The compositions will be provided in different vessels, vehicles or formulations depending upon the disorder and mode of administration. For example, and as described in greater detail herein, for oral application, the compounds can be administered as sublingual tablets, gums, mouth washes, toothpaste, candy, gels, films, etc.; for ocular application, as eye drops in eye droppers, eye ointments, eye gels, eye packs, as a coating on a contact lens or an intraocular lens, in contacts lens storage or cleansing solutions, etc.; for topical application, as lotions, ointments, gels, creams, sprays, tissues, swabs, wipes, etc.; for vaginal or rectal application, as an ointment, a tampon, a suppository, a mucoadhesive formulation, etc.

A variety of other administration routes are also available. The particular mode selected will depend, of course, upon the particular active agent(s) selected, the desired results, the particular condition being treated and the dosage required for therapeutic efficacy. The methods of this invention, generally speaking, may be practiced using any mode of administration that is medically acceptable, meaning any mode that produces effective levels of inflammatory response alteration without causing clinically unacceptable adverse effects. One mode of administration is the parenteral route. The term “parenteral” includes subcutaneous injections, intravenous, intramuscular, intraperitoneal, intrasternal injection or infusion techniques. Other modes of administration include oral, mucosal, rectal, vaginal, sublingual, intranasal, intratracheal, inhalation, ocular, transdermal, etc. In some embodiments the administration of the compositions does not occur via the pulmonary route. In other embodiments the administration is intravenous, subcutaneous or by inhalation.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

One suitable oral form is a sublingual tablet. A sublingual tablet delivers the composition to the sublingual mucosa. As used herein, “tablet” refers to pharmaceutical dosage forms prepared by compressing or molding. Sublingual tablets are small and flat, for placement under the tongue and designed for rapid, almost instantaneous disintegration and release the composition to the sublingual mucosa. The term “disintegration” means breaking apart. Preferably, the sublingual tablets of the present invention disintegrate, to release the composition, within five minutes and, more preferably, within a two minute period of time. Oral formulations can also be in liquid form. The liquid can be administered as a spray or drops to the entire oral cavity including to select regions such as the sublingual area. The sprays and drops of the present invention can be administered by means of standard spray bottles or dropper bottles adapted for oral or sublingual administration. The liquid formulation is preferably held in a spray bottle, fine nebulizer, or aerosol mist container, for ease of administration to the oral cavity. Liquid formulations may be held in a dropper or spray bottle calibrated to deliver a predetermined amount of the composition to the oral cavity. Bottles with calibrated sprays or droppers are known in the art. Such formulations can also be used in nasal administration.

The compositions of the invention can also be formulated as oral gels. As an example, the composition may be administered in a mucosally adherent, non-water soluble gel. The gel is made from at least one water-insoluble alkyl cellulose or hydroxyalkyl cellulose, a volatile nonaqueous solvent, and the composition. Although a bioadhesive polymer may be added, it is not essential. Once the gel is contacted to a mucosal surface, it forms an adhesive film due primarily to the evaporation of the volatile or non-aqueous solvent. The ability of the gel to remain at a mucosal surface is related to its filmy consistency and the presence of non-soluble components. The gel can be applied to the mucosal surface by spraying, dipping, or direct application by finger or swab.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Medical devices for the inhalation of therapeutics are known in the art. In some embodiments the medical device is an inhaler. In other embodiments the medical device is a metered dose inhaler, diskhaler, Turbuhaler, diskus or a spacer. In certain of these embodiments the inhaler is a Spinhaler (Rhone-Poulenc Rorer, West Malling, Kent). Other medical devices are known in the art and include the following technologies Inhale/Pfizer, Mannkind/Glaxo and Advanced Inhalation Research/Alkermes.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. In some embodiments the compounds provided are administered by infusion pump. In some of these embodiments the compounds are administered by infusion pump. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990 and Langer and Tirrell, Nature, 2004 Apr. 1; 428(6982): 487-92, which are incorporated herein by reference.

The compositions may conveniently be presented in unit dosage form and may be prepared by any of the methods well known in the art of pharmacy.

In some embodiments the composition that is administered is in powder or particulate form rather than as a solution. Examples of particulate forms contemplated as part of the invention in some embodiments are provided in U.S. patent application Ser. No. 09/982,548, filed Oct. 18, 2001, which is hereby incorporated by reference in its entirety. In other embodiments the compositions are administered in aerosol form. In other embodiments the method of administration includes the use of a bandage, slow release patch, engineered or biodegradable scaffold, slow release polymer, tablet or capsule.

Other delivery systems can include time-release, delayed release or sustained release delivery systems. Such systems can avoid repeated administrations of the compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the agent is contained in a form within a matrix, found in U.S. Pat. Nos. 4,452,775 (Kent); 4,667,014 (Nestor et al.); and 4,748,034 and 5,239,660 (Leonard) and (b) diffusional systems in which an agent permeates at a controlled rate through a polymer, found in U.S. Pat. Nos. 3,832,253 (Higuchi et al.) and 3,854,480 (Zaffaroni). In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable and bioerodable/biodegradable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate. In one embodiment the slow release polymer is a block copolymer, such as poly(ethylene glycol) (PEG)/poly(lactic-co-glycolic acid) (PLGA) block copolymer.

Examples of preferred non-biodegradable polymers include ethylene vinyl acetate, poly(meth) acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

In another embodiment slow release is accomplished with the use of polyanhydride wafers.

The compositions can be administered locally or the compositions can further include a targeting molecule. The targeting molecule can be attached to the agent and/or the additional therapeutic agent or some combination thereof. A targeting molecule is any molecule or compound which is specific for a particular cell or tissue and which can be used to direct the agents provided herein to a particular cell or tissue. The targeted molecules can be any molecule that is differentially present on a particular cell or in a particular tissue. These molecules can be proteins expressed on the cell surface.

Targeting molecules can in some embodiments be used to target disease markers. For instance, the targeting molecule may be a protein (e.g., an antibody) or other type of molecule that recognizes and specifically interacts with a disease antigen. The targeting molecule, therefore, may be a molecule that targets a protein or other type of molecule that recognizes and specifically interacts with a tumor antigen.

Tumor-antigens include Melan-A/MART-1, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, am11, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pmell117}, PRAME, NY-ESO-1, brain glycogen phosphorylase, SSX-1, SSX-2 (HOM-MEL-40), SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, Smad family of tumor antigens, 1mp-1, EBV-encoded nuclear antigen (EBNA)-1, and c-erbB-2.

Also provided, therefore, are GAGs linked to a targeting molecule as well as compositions thereof and methods of their use.

Some aspects of the invention also encompass kits. The kits of the invention include one or more of the agents of the invention. The kits can further include one or more additional therapeutic agents, administration devices and/or instructions for use. The kits provided can also include a detection system. Detection systems can be used to determine the amount of any or all of the agents administered in the blood. Detection systems can be invasive or non-invasive. An example of an invasive detection system is one which involves the removal of a blood sample and can further involve an assay such as an enzymatic assay or a binding assay to detect levels in the blood. A non-invasive type of detection system is one which can detect the levels of the agent in the blood without having to break the skin barrier. These types of non-invasive systems include, for instance, a monitor which can be placed on the surface of the skin, e.g., in the form of a ring or patch, and which can detect the level of circulating agents. One method for detection may be based on the presence of fluorescence in the agent which is administered. Thus, if a fluorescently labeled agent is administered and the detection system is non-invasive, it can be a system which detects fluorescence. This is particularly useful in the situation when the patient is self-administering and needs to know the blood concentration or an estimate thereof in order to avoid side effects or to determine when another dose is required.

A subject is any human or non-human vertebrate, e.g., dog, cat, horse, cow, monkey, pig, mouse, rat.

The present invention is further illustrated by the following Examples, which in no way should be construed as further limiting. The entire contents of all the references (including literature references (except those only listed in the Reference list below), issued patents, published patent applications, and co-pending patent applications) cited throughout this application are herein incorporated by reference.

### EXAMPLES

### Heparan Sulfate and Dermatan Sulfate Glycosaminoglycans Regulate Fibroblast Growth Factor and Vascular Endothelial Growth Factor Activity

### Materials and Methods

### Proteins and Reagents

FBS was from Hyclone (Logan, Utah). L-glutamine, penicillin/streptomycin, PBS and Trizol reagent were from GibcoBRL (Gaithersberg, Md.). Unfractionated heparin, HS, UDS, and DS DT were from Celsus Laboratories (Cincinnati, Ohio); diDS and ddDS were produced as described [45, 226]. CS A and CS C were from Sigma (St. Louis, Mo.). CS D and CS E were from Celsus laboratories. Recombinant FGF1 was a gift from Amgen (Thousand Oaks, Calif.). Recombinant human FGF2 was a gift from Scios (Mountainview, Calif.). Recombinant FGF7 and VEGF₁₆₄ were from Sigma. Rabbit α-Akt1/2, rabbit α-phospho-Akt1/2/3 (Ser 473), rabbit α-phospho-Akt1/2/3 (Thr 308), rabbit α-VEGF, rabbit α-VEGF-C, rabbit α-VEGF-D, goat α-VEGFR2/Flk-1, rabbit α-VEGFR3/Flt-4, rabbit α-Erk1, rabbit α-Erk2, goat α-phospho-Erk1/2 (Thr 202/Tyr 204), rabbit α-Mek1, rabbit α-Mek2, goat α-phospho-Mek1/2 (Ser 218/Ser 222), rabbit α-goat conjugated to horseradish peroxidase (HRP) and goat α-rabbit conjugated to HRP were from Santa Cruz Biotechnology (Santa Cruz, Calif.).

### Cell Culture

NBT-II cells (American Type Culture Collection, Manassas, Va.) were maintained in minimum essential medium (American Type Culture Collection) supplemented with 1.5 mg/mL sodium bicarbonate, 0.1 mM non-essential amino acids, 1.0 mM sodium pyruvate, 100 μg/ml penicillin, 100 U/ml streptomycin, 500 μg/ml L-glutamine and 10% FBS. Cells were grown in 75 cm² flasks at 37° C. in a 5% CO₂ humidified incubator. Confluent cultures were split 1:5 to 1:10, two times per week.

### Proliferation Assays

NBT-II cells were grown until confluence in 75 cm² flasks. Each flask was washed with 20 ml PBS and treated with 3 ml trypsin-EDTA at 37° C. for ˜15 minutes until cells completely detached. Cells were centrifuged for 3 minutes at 195×g. The supernatant was aspirated, and the cells were resuspended in 10 ml media. Cell density was measured using an electronic cell counter, and the suspension was diluted to 50,000 cells/ml. The suspension was plated 1 ml/well into 24-well tissue culture plates. After a 24 hour incubation in a 5% CO₂, 37° C. humidified incubator, the media was aspirated, the wells were washed with serum free media, and the cells were supplemented with media containing 0.1% FBS and incubated for 24 hours. Cells were sequentially treated with antibodies, GAGs and growth factors as appropriate. Sodium chlorate was added at 50 mM [30]. Antibodies to VEGFR2 or VEGFR3 were added to yield a final dilution of 1:100. All GAGs were initially added over a range of concentrations from 1 ng/ml to 100 μg/ml. Heparin, UDS and DS DT were subsequently added at 1 μg/ml unless otherwise noted. FGF1, FGF2, FGF7 and VEGF were added at 10 ng/ml unless otherwise noted. Cells were then incubated for 72 hours. Wells were then washed twice with PBS and treated with 0.5 ml trypsin-EDTA/well and incubated for 10 minutes at 37° C. Whole cell number was determined using an electronic cell counter.

### RT-PCR

Five μg of total RNA was isolated from NBT-II cells using Trizol reagent (Life Tech, Rockville, Md.) followed by reverse transcription with random hexamers. Specific oligomers were designed based on the published sequences of FGFR isoforms in order to detect their expression. Sequences of primer pairs corresponding to distinct FGFR isoforms were as follows: FGFR1b: 5′-TGG AGC AAG TGC CTC CTC-3′ (SEQ ID NO: 1) and 5′-ATA TTA CCA CTT CGA TTG GTC-3′ (SEQ ID NO: 2); FGFR1c: 5′-TGG AGC TGG AAG TGC CTC CTC-3′ (SEQ ID NO: 3) and 5′-GTG ATG GGA GAG TCC GAT AGA-3′ (SEQ ID NO: 4); FGFR2b: 5′-GTC AGC TGG GGT CGT TTC ATC-3′ (SEQ ID NO: 5) and 5′-CTG GTT GGC CTG CCC TAT ATA-3′ (SEQ ID NO: 6); FGFR2c: 5′-GTC AGC TGG GGT CGT TTC ATC-3′ (SEQ ID NO: 7) and 5′-GTG AAA GGA TAT CCC AAT AGA-3′ (SEQ ID NO: 8); FGFR3b: 5′ GTA GTC CCG GCC TGC GTG CTA-3′ (SEQ ID NO: 9) and 5′-GAC CGG TTA CAC AGC CTC GCC-3′ (SEQ ID NO: 10); FGFR3c: 5′-GTA GTC CCG GCC TGC GTG CTA-3′ (SEQ ID NO: 11) and 5′-TCC TTG CAC AAT GTC ACC TTT-3′ (SEQ ID NO: 12); and FGFR4: 5′-CCC TGC CGG GAT CGT GAC CCG-3′ (SEQ ID NO: 13) and 5′-TCG AAG CCG CGG CTG CCA AAG-3′ (SEQ ID NO: 14). Sequences of primer pairs corresponding to distinct VEGFR isoforms were as follows: VEGFR1: 5′-CGG ACA CTC CCG GGA GGT AGT-3′ (SEQ ID NO: 15) and 5′-CTT CTG TCG AGT AGG GGA-3′ (SEQ ID NO: 16); VEGFR2: 5′-TGC GGG CCA GGG ACG GAG AAG-3′ (SEQ ID NO: 17) and 5′-CTA GTT ACT ACT TTG GAT AGT-3′ (SEQ ID NO: 18); and VEGFR3: 5′-CGG GCG CTG CGC TGA ACC GGC-3′ (SEQ ID NO: 19) and 5′-TCG ACA TGG GGT TCT TCA GTG-3′ (SEQ ID NO: 20). To control for total cell protein, RT-PCR was also performed on β-actin using the primers 5′-GCC AGC TCA CCA TGG ATG ATG ATA T-3′ (SEQ ID NO: 21) and 5′-GCT TGC TGA TCC ACA TCT GCT GGA A-3′ (SEQ ID NO: 22). PCR was performed using the Advantage-GC cDNA kit from Clontech as per manufacturer's instructions (Palo Alto, Calif.). Prior to experimental use, primers were confirmed to detect and have specificity towards given receptor isoforms.

### Whole Cell ELISA

ELISA was performed using whole cells to quantify relative levels of kinase activity. NBT-II cells were grown until confluence in 75 cm² flasks. Each flask was washed with 20 ml PBS and treated with 3 ml trypsin-EDTA at 37° C. for 3-5 minutes, until cells detached. Cells were centrifuged for 3 minutes at 195×g. The supernatant was aspirated, and the cells were resuspended in 10 ml media. The cell density was measured using an electronic cell counter, and the suspension was diluted to 50,000 cells/ml. 100 mm dishes were supplemented with 10 ml cell suspension per dish. After a 24 hour incubation, the media was aspirated, the dishes washed with serum free media, and the cells supplemented with media containing 0.1% FBS. After a 24 hour incubation, dishes were treated with PBS, 10 μg/ml heparin or 10 μg/ml DS DT. Subsequently, cells were treated with 10 ng/ml FGF7, 10 ng/ml VEGF or both. Cells were incubated for 30 minutes (for Erk1, Erk2, phosphor-Erk1/2, Mek1, Mek2, phospho-Mek1/2, Akt1/2 and phospho-Akt1/2/3) or 24 hours (for VEGF, VEGF-C and VEGF-D). Media were aspirated and cells were homogenized per manufacture instructions. Total protein concentration was determined by Bradford assay. An equivalent protein concentration from cell extract was added to 96-well plates previously incubated for 1 hour with primary antibodies to Erk1, Erk2, phospho-Erk1/2, Mek1, Mek2, phospho-Mek1/2, Akt1/2, phospho-Akt1/2, VEGF, VEGF-C or VEGF-D. The cell extract was incubated on the plates for 1 hour, after which wells were washed twice and supplemented with the same primary antibody (1:100) as was in the well. Wells were incubated 1 hour, washed twice, and treated with HRP-conjugated secondary antibody (1:500). Plates were incubated for 30 minutes, washed twice, and incubated with TMB One Solution (Promega, Madison, Wis.). The reaction was quenched with 3 M sulfuric acid, and the plates were analyzed using a UV plate reader at 450 nm. Data were quantified by comparing to a standardized curve with varying concentrations of protein from untreated cells.

### Results

### Heparin and DS DT Support FGF7-Mediated Responses

Studies exploring the interactions between GAGs and FGFs are typically confined to the binding of heparin and other HSGAGs to FGF, and subsequent downstream responses. Recent findings have demonstrated, however, that DS can also bind to and modulate the activities of both FGF2 and FGF7 [366, 475]. The differential effects of various GAGs on growth factor signaling was examined. The Nara bladder tumor No. 2 (NBT-II) cell line, previously demonstrated to respond to various FGFs and to express FGFR2b, necessary for FGF7-mediated proliferation [36, 348, 354], was used. Dose response curves revealed that FGF7 elicits its maximal effect on cell growth in NBT-II cells at 5 ng/ml. The magnitude of this effect remains constant through 100 ng/ml. The maximal proliferative effect, however, was not achieved until 10 ng/ml in the presence of 50 mM sodium chlorate.

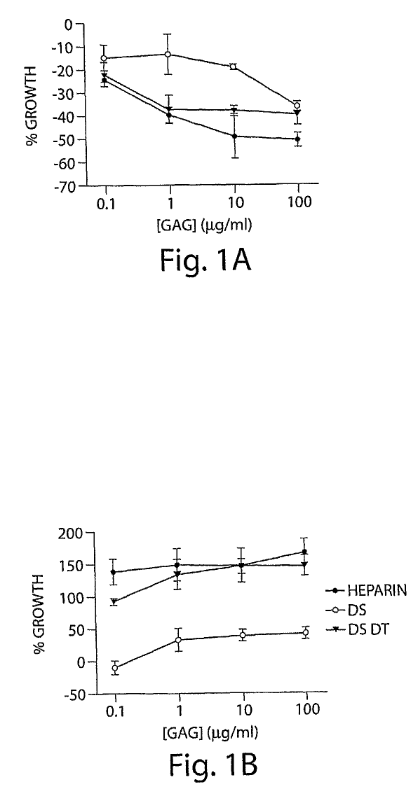

Each of heparin, HS, CS A, CS C, unfractionated DS (UDS) and DS DT were added at various concentrations to NBT-II cells, along with 10 ng/ml FGF7. The addition of GAG alone had no effect on whole cell proliferation. In the presence of FGF7, GAGs showed differential capacities to modulate the FGF7-mediated response (FIG. 1), both in the presence and absence of sodium chlorate. Heparin and DS DT were the most potent and efficacious of the GAGs, promoting 51.2±3.0% and 40.2±4.5% reductions in whole cell number, respectively, and 165.6±21.6% and 145.8±14.9% increases in whole cell number respectively in the presence of chlorate. FGF7 alone induced a 14.1±2.5% reduction and 28.4±11.8% increase in whole cell number untreated with and treated with sodium chlorate, respectively.

### Heparin and DS DT Modulate FGF1-, FGF2- and VEGF-Mediated Effects

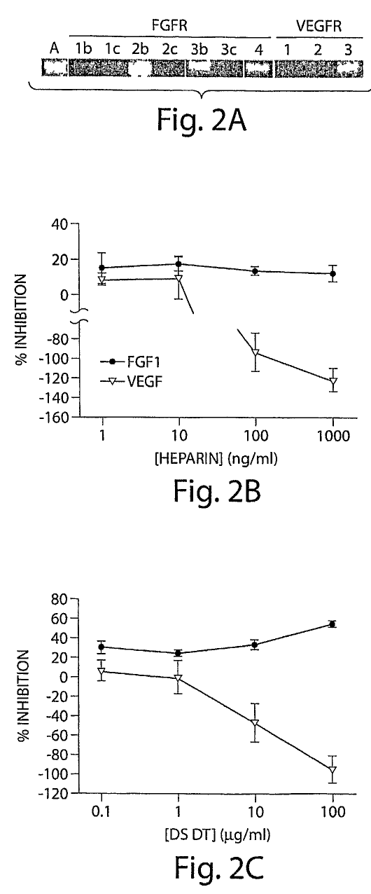

The modulatory capacity of GAGs on other growth factors was examined. NBT-II cells have been previously demonstrated to support FGF1, FGF2 and VEGF signaling [36]. RT-PCR was performed to verify that NBT-II cells expressed receptors to support the responses of these ligands. Cells clearly expressed FGFR2b, FGFR3b, FGFR4 and VEGFR3 (FIG. 2A). Lower levels of VEGFR2 were observed. FGF2 and VEGF reduced whole cell number (Table 1), while FGF1 did not induce significant proliferative effects in the absence of GAGs.

TABLE 1
Inhibitory effects of growth factors

| | PBS | FGF7 |
|---|---|---|
| PBS | 0.0 ± 5.6 | 14.1 ± 2.5 |
| FGF1 | 5.4 ± 8.3 | 18.0 ± 2.6 |
| FGF2 | 18.3 ± 5.0 | 30.4 ± 8.7 |
| VEGF | 19.8 ± 4.5 | 30.1 ± 7.0 |

Column and row heading represent the addition of ligand (at 10 ng/ml) or PBS. Numbers represent percent reduction in whole cell number ± standard deviation.

### Heparin and DS DT Differentially Regulate Growth Factor Function

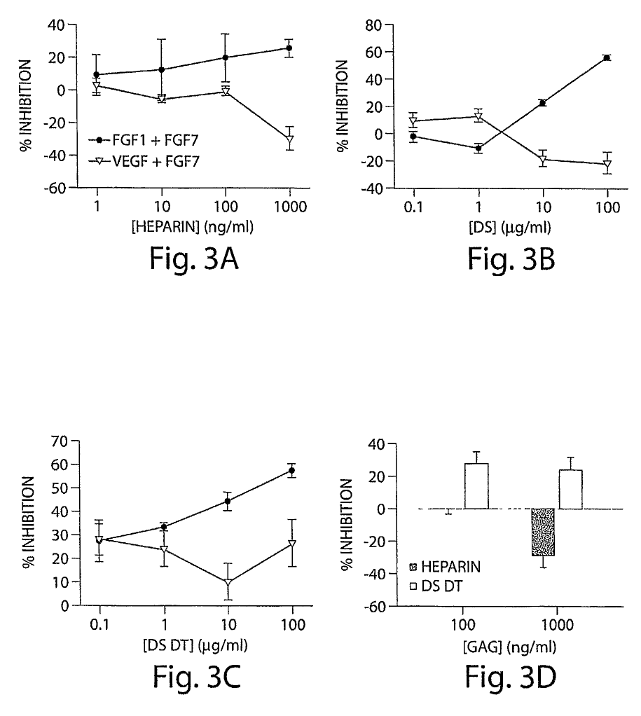

The most pronounced growth modulatory effects induced by GAGs were exhibited with FGF7 and VEGF. The cellular response with the co-administration of multiple ligands was then explored. The addition of FGF7 with FGF1, FGF2 or VEGF reduced whole cell number in an additive manner (Table 1). The addition of GAGs, however, substantially changed the observed response. Heparin with FGF1+FGF7 reduced whole cell number by 25.9±0.6% compared to the ligands only (FIG. 3A). Heparin did not alter the effects of FGF2+FGF7. Heparin with VEGF+FGF7 increased whole cell number 29.5±7.1% compared to the ligands only. The addition of UDS (FIG. 3B) led to a greater reduction in whole cell number for FGF1+FGF7, but did not have effects distinct from heparin, for either FGF2+FGF7 or VEGF+FGF7. DS DT (FIG. 3C) had a similar effect as UDS on FGF1+FGF7, reducing whole cell number 57.2±3.0% relative to the ligand combination, but showed a unique response with VEGF+FGF7, reducing whole cell number 26.5±10.0% compared to the ligand combination. Heparin and DS DT at 1 μg/ml therefore show unique capacities to regulate VEGF+FGF7 (FIG. 3D), with heparin promoting proliferation and DS DT inhibiting it.

### FGF7 and VEGF Utilize Different Signaling Cascades

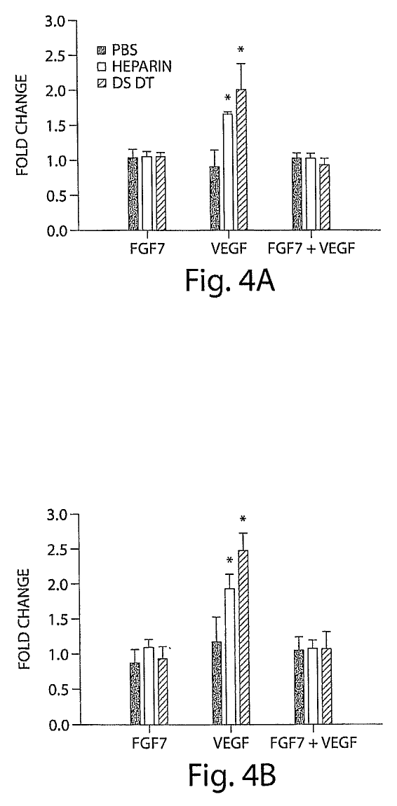

Heparin and DS DT both inhibit proliferation in the presence of FGF7 and support proliferation in the presence of VEGF. In the presence of both ligands, the two GAGs unveil distinct effects. The signal cascades activated by the ligands supplemented with PBS, heparin and DS DT was, therefore, examined. VEGF increased phosphorylated Erk1/2 and Mek1/2 when treated with heparin or DS DT (FIG. 4). No changes in Erk1, Erk2, Mek1or Mek2 levels were observed with any ligand-GAG combination tested. Erk1/2 phosphorylation was increased 1.65±0.02-fold with heparin (p<0.0004) and 2.01±0.36-fold with DS DT (p<0.02). Mek1/2 phosphorylation was increased 1.92±0.21-fold with heparin (p<0.002) and 2.47±0.25-fold with DS DT (p <0.0004). When FGF7 was present along with VEGF and heparin or DS DT, however, the increase in Erk1/2 and Mek1/2 phosphorylation was abrogated.

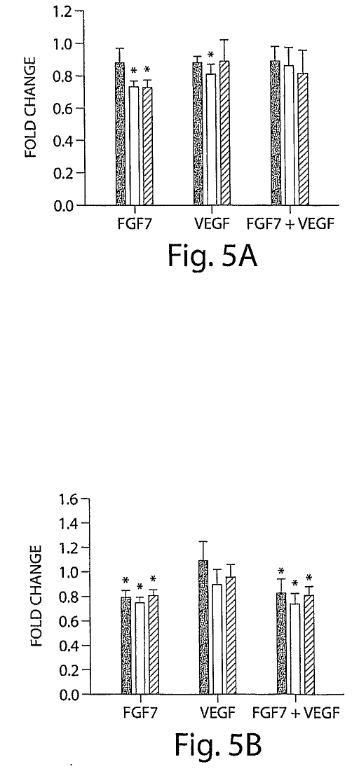

While changes in Erk1/2 and Mek1/2 phosphorylation were consistent with cellular responses to VEGF in the presence of heparin or DS DT, they did not reflect the changes induced by FGF7, unsupplemented VEGF or by VEGF+FGF7. To this end, induction of Akt1/2/3 phosphorylation was examined. Levels of Akt1/2 were not affected by any ligand-GAG combination. FGF7 in the presence of either heparin (27.8±13.8%; p<0.005) or DS DT (27.4±4.6%; p<0.004) reduced phosphorylation of Akt1/2/3 (Ser 473; FIG. 5A). FGF7 and VEGF+FGF7 also reduced phosphorylation of Akt1/2/3 (Thr 308; FIG. 5B) ˜20% in the presence of PBS, heparin or DS DT.

### Upregulated VEGF-D is Responsible for the Distinct Modulatory Capacities of Heparin and DS DT

The changes in Erk1/2, Mek1/2 and Akt1/2/3 phosphorylation were consistent with the effects of FGF7 or VEGF in the presence of PBS, heparin or DS DT, as observed by whole cell counts. The results, however, were not sufficient to explain the effects observed with FGF7 and VEGF together. The receptors responsible for the differential effects of heparin and DS DT on FGF7+VEGF were, therefore, defined. Blocking VEGFR2 with a neutralizing antibody produced a VEGF+FGF7 response similar to FGF7, consistent with the VEGF response being dependent on VEGFR2. Correspondingly, blocking FGFR2, through which FGF7 signals [348], produced a VEGF+FGF7 response similar to VEGF alone. Blocking VEGFR3 did not alter either FGF7- or VEGF-mediated responses, but surprisingly eliminated the capacity of heparin and DS DT to modulate the effects of the ligands when co-administered.

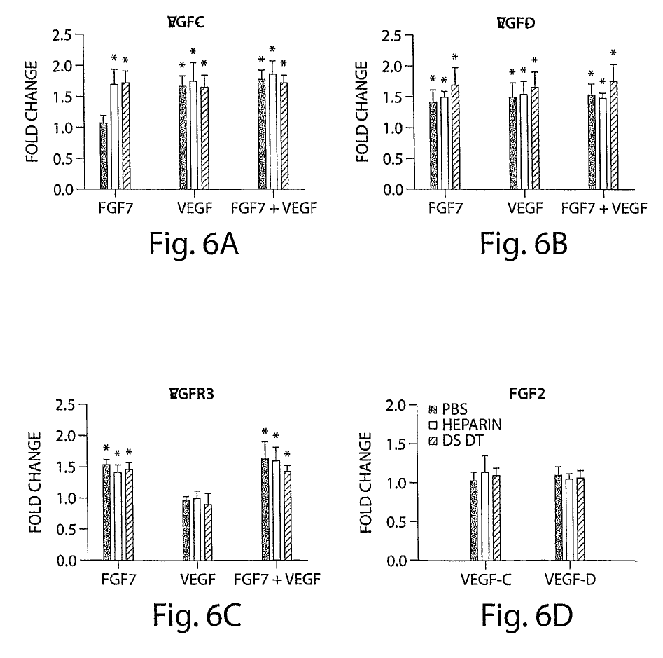

VEGFR3 supports signaling from VEGF-C and VEGF-D [249]. Therefore, the potential source of VEGF-C and/or VEGF-D was investigated. The ability of FGF7 and VEGF in the presence of GAGs to increase levels of VEGF-C and VEGF-D was examined over 24-hours. VEGF-C levels were increased by VEGF regardless of GAG used, FGF7 in the presence of heparin or DS DT, and VEGF+FGF7 regardless of the GAG used (FIG. 6A). VEGF-D levels were elevated by all combinations of FGF7, VEGF and GAG (FIG. 6B). Interestingly, addition of FGF7, but not VEGF, caused an increase in VEGFR3 production (FIG. 6C). FGF2 did not alter the production of VEGF-C or VEGF-D (FIG. 6D), suggesting that the effect is ligand specific.

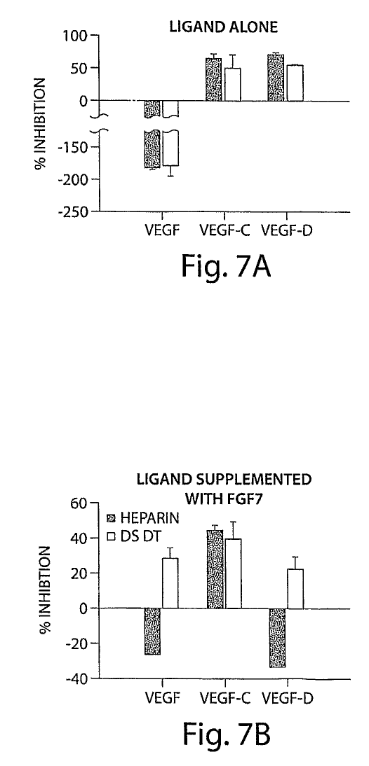

The capacity of VEGF-C and VEGF-D to promote NBT-II proliferation was subsequently investigated. VEGF alone reduced cell number 19.8±4.5%, and 30.1±7.0% in the presence of FGF7. VEGF-C alone similarly reduced cell number 13.4±8.7% (p<0.05 compared to untreated cells), but only 5.9±5.0% in the presence of FGF7 (p>0.18 compared to untreated cells). VEGF-D alone reduced cell number 16.2±10.8% (p<0.05 compared to untreated cells), and 34.5±1.5% in the presence of FGF7 (p<0.0004 compared to untreated cells). Whether heparin and DS DT could modulate VEGF-C and VEGF-D signaling alone and in the presence of FGF7 was then explored. The addition of heparin and DS DT with VEGF-C or VEGF-D reduced whole cell number more than either ligand alone (FIG. 7A). The capacity of heparin and DS DT to modulate VEGFs+FGF7 was subsequently examined. Heparin promoted a similar increase in whole cell number for VEGF+FGF7 and VEGF-D+FGF7 relative to ligands only (FIG. 7B). DS DT promoted a similar reduction in whole cell number for both VEGF+FGF7 and VEGF-D+FGF7 relative to ligands only.

### Oversulfated DS Species Promote Greater Cellular Mediated Responses

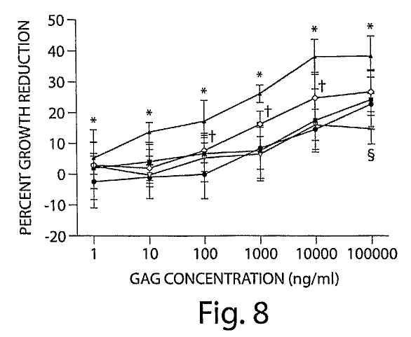

The ability of the oversulfated DS DT to selectively induce a FGF7-like response when mixed with other growth factors led us to examine the effects of chemically oversulfated GAGs on FGF7 activity. CS D, CS E, chemically oversulfated DS DT (diDS) and doubly chemically oversulfated DS DT (ddDS), are CS and DS species with increased degrees of sulfation compared to other similar GAGs examined [45, 226]. The ability of these species to alter FGF7 cellular mediated responses was examined in comparison to DS DT. When normalized to the effects of FGF7, 100 μg/ml DS DT reduced whole cell number 22.7±3.6% (FIG. 8). CS D elicited a smaller magnitude of response at 100 μg/ml (15.0±5.4% p<0.03), but showed no difference at any other concentration examined. The effects of CS E were not significantly different than DS DT at any concentration. The similarities between the effects induced by oversulfated CS species and DS DT are notable as while CS A and CS C did not support FGF7-mediated effects as efficaciously as DS DT, the CS species with increased sulfation induced a greater magnitude of response. Similarly, in the presence of FGF7, diDS reduced whole cell number greater than DS DT at 100 ng/ml (p<0.03), 1 μg/ml (p<0.008) and 10 μg/ml (p<0.03), but the difference was absent at 100 μg/ml. 10 μg/ml diDS had a similar effect (24.8±8.0%), however, to 100 μg/ml DS DT, demonstrating an increase in potency. The addition of a DS species with even higher sulfation, ddDS produced a response that was significantly greater than that elicited with DS DT at each and every concentration examined (p<0.03).

### Discussion

DS and heparin, but not CS, have been previously demonstrated to modulate FGF7 signaling in cells lacking surface GAGs as well as normal keratinocytes [475]. Herein, analysis was extended to pathological cells. NBT-II cells express FGFR2b, the receptor for FGF7 [348] and have cell surface GAGs, as evidenced by the change in cellular response to FGF7 and various GAGs after sodium chlorate treatment, which abrogates cell surface HSPGs [448]. While heparin and DS DT promoted maximal cellular mediated responses, species from each of HSGAGs, CS GAGs and DS notably regulated FGF7 activity in cancer cells. CS C importantly and specifically supported substantial FGF7-induced responses, albeit to a lower degree than either heparin or DS DT. These results demonstrate that specific CS fractions can therefore support FGF7 activity. The specific role of CS C in promoting FGF7 mediated cell proliferation, however, is not clear. CS has been demonstrated to upregulate FGF7 production [419], which could account for the increased cellular-mediated response observed, although sufficient FGF7 to induce the maximal response in the absence of exogenous GAG was added at the outset of the experiment. As such, this report provides the first evidence of CS C modulating FGF7-mediated responses.

Given that specific fractions of all GAG families examined could promote FGF7 activity, this analysis was extended to other FGFs and the VEGF family. FGF1 and FGF2 were chosen based on the FGFR isoform expression profile of NBT-II cells, as well as their previously demonstrated role in defining NBT-II growth and progression [36]. VEGF was used given its important role in bladder cancer growth [506]. Heparin and DS DT, which promoted equivalent FGF7-mediated activities that were greater than all other GAGs examined, modulated each of FGF1, FGF2, FGF7 and VEGF cellular mediated responses. The strong regulatory capacity observed with DS DT demonstrates that DS species can in fact impact members of the FGF family, such as FGF1. Additionally, DS can regulate the activity of VEGF, whose interactions with DS had previously not been examined. DS may also regulate FGF2 activity through FGFR3c and/or FGFR4, in addition to FGFR1c, the isoform previously associated with DS-FGF2 interactions [366] given the observed response in cells lacking FGFR1c.

Heparin and DS DT modulated VEGF-induced responses to promote substantial proliferation while VEGF alone led to growth inhibition. This finding was unique to VEGF, as the addition of exogenous GAGs enhanced the inhibitory capacity of the FGFs examined. VEGF in the presence of GAGs promoted Erk1/2 and Mek1/2 phosphorylation, unlike VEGF alone or FGF7, consistent with the observed proliferative effects [453]. Heparin is essential for the activity of certain VEGF isoforms to promote cellular responses [113]. The growth inhibitory effects of FGF7 and VEGF, however, appear to be Akt-mediated. In addition to merely modulating ligand activity, heparin and DS DT elicit distinct patterns of cellular response from multiple ligands. Heparin with VEGF+FGF7 had a proliferative response while DS DT with VEGF+FGF7 had an inhibitory one. The unique patterns of response suggest that these two GAGs can be used to initiate specific cellular responses in a complex mix of growth factors, such as that which exists in the ECM. Altering the GAG composition of the ECM may therefore be a mechanism that cells use to change biological activities in response to various environmental cues.

The cellular pathways by which heparin and DS DT elicit distinct cellular responses are important in order to understand their effects. The cellular activities of VEGF are altered in the presence of FGF7. Unlike VEGF supplemented with GAG, Erk1/2 and Mek1/2 were not phosphorylated in response to VEGF+FGF7. Further, VEGF signaled through VEGFR2, with neutralizing antibodies eliminating its effect. Though the combined VEGF+FGF7 response was dependent on VEGFR3, suggesting the involvement of VEGF-C and/or VEGF-D [249]. Each of FGF7, VEGF and VEGF+FGF7 promoted VEGF-C and VEGF-D activity in the presence of GAGs. The cellular response to VEGF-D was additionally modulated by heparin and DS DT in the same manner as VEGF+FGF7. Therefore, the differential regulation of VEGF+FGF7 by heparin and DS DT is based on the upregulation of VEGF-D production and subsequent modulation of its activity, mediated by VEGFR3.

The distinct cellular responses obtained with heparin and DS DT stem primarily from differential regulation of VEGF-D. Heparin and DS DT affect VEGF-mediated cellular activity in a similar manner. Their relative regulatory capacities are, however, distinct between various VEGFs. Various GAGs may, therefore, be important physiological and pathological regulators of VEGF.

The results presented herein demonstrate that specific GAG fractions beyond heparin can serve a regulatory role for several growth factors. The highly sulfated heparin modulated the response to all growth factors examined. The highly sulfated dermatan sulfate fraction DS DT elicited a similar ability to affect the growth factors examined with comparable magnitudes but a distinct net effect from heparin. CS additionally promoted FGF7 activity. Interestingly, increasing the sulfation of CS and DS species supported higher levels of FGF7 activity than corresponding GAGs with lower degrees of sulfation. These findings demonstrate that the ability of GAGs to regulate FGFs, VEGFs and mixtures of growth factors, extend well beyond those of HSGAGs. As heparin and DS can promote selective cellular activities in a mixture of growth factors, the development of chemically oversulfated species such as ddDS can further enable controlled growth factor activity and specification of cellular behavior. The selectivity of highly sulfated DS species for FGF7 activity and the increased magnitude of response elicited by ddDS suggests that it may be an important new therapeutic (e.g., wound healing, cancer), especially in the complex environment created by the physiological response to insult.

### REFERENCES

The listing of the references in the following list is not intended to be an admission that any of the references is a prior art reference.
1. Abantangelo, G., and Weigel, P. (2000). New frontiers in medical science: redefining hyaluron. (Amsterdam: Elsevier).
2. Achen, M. G., Jeltsch, M., Kukk, E., Makinen, T., Vitali, A., Wilks, A. F., Alitalo, K., and Stacker, S. A. (1998). Vascular endothelial growth factor D (VEGF-D) is a ligand for the tyrosine kinases VEGF receptor 2 (Flk1) and VEGF receptor 3 (Flt4). Proc Natl Acad Sci USA 95, 548-553.
3. Adams, D. H., and Shaw, S. (1994). Leucocyte-endothelial interactions and regulation of leucocyte migration. Lancet 343, 831-836.
4. Akinc, A., Lynn, D. M., Anderson, D. G., and Langer, R. (2003). Parallel synthesis and biophysical characterization of a degradable polymer library for gene delivery. J Am Chem Soc 125, 5316-5323.
5. Alexander, C. M., Reichsman, F., Hinkes, M. T., Lincecum, J., Becker, K. A., Cumberledge, S., and Bernfield, M. (2000). Syndecan-1 is required for Wnt-1-induced mammary tumorigenesis in mice. Nat Genet 25, 329-332.
6. Allen, B. L., Filla, M. S., and Rapraeger, A. C. (2001). Role of heparan sulfate as a tissue-specific regulator of FGF-4 and FGF receptor recognition. J Cell Biol 155, 845-858.
7. Anderson, D. G., Lynn, D. M., and Langer, R. (2003). Semi-automated synthesis and screening of a large library of degradable cationic polymers for gene delivery. Angew Chem Int Ed 42, 3153-3158.
8. Anderson, D. G., Peng, W., Akinc, A., Hossain, N., Kohn, A., Padera, R., Langer, R., and Sawicki, J. A. (2004). A polymer library approach to suicide gene therapy for cancer. Proc Natl Acad Sci USA 101, 16028-16033.
9. Anderson, J. M., Van Itallie, C. M., Peterson, M. D., Stevenson, B. R., Carew, E. A., and Mooseker, M. S. (1989). ZO-1 mRNA and protein expression during tight junction assembly in Caco-2 cells. J Cell Biol 109, 1047-1056.
10. Anderson, W. F. (1998). Human gene therapy. Nature 392, 25-30.
11. Antman, E. M., and Handin, R. (1998). Low-molecular-weight heparins: an intriguing new twist with profound implications. Circulation 98, 287-289.
12. Arfors, K. E., Lundberg, C., Lindbom, L., Lundberg, K., Beatty, P. G., and Harlan, J. M. (1987). A monoclonal antibody to the membrane glycoprotein complex CD18 inhibits polymorphonuclear leukocyte accumulation and plasma leakage in vivo. Blood 69, 338-340.
13. Arnott, S., Mitra, A. K., and Raghunathan, S. (1983). Hyaluronic acid double helix. J Mol Biol 169, 861-872.
14. Ashikaga, T., Strada, S. J., Thompson, W. J. (1997). Altered expression of cyclic nucleotide phosphodiesterase isozymes during culture of aortic endothelial cells. Biochem Pharmacol 54, 1071-1079.
15. Ay, I., Sugimori, H., and Finklestein, S. P. (2001). Intravenous basic fibroblast growth factor (bFGF) decreases DNA fragmentation and prevents downregulation of Bcl-2 expression in the ischemic brain following middle cerebral artery occlusion in rats. Brain Res Mol Brain Res 87, 71-80.
16. Baird, A. (1994). Fibroblast growth factors: activities and significance of non-neurotrophin neurotrophic growth factors. Curr Opin Neurobiol 4, 78-86.
17. Balazs, E. A., and Denlinger, J. L. (1989). Clinical uses of hyaluronan: the biology of hyaluronan. In Clinical uses of hyaluronan: the biology of hyaluronan, D. Evered and J. Welan, eds. (New York: Wiley), pp. 265-280.
18. Ballinger, M. D., Shyamala, V., Forrest, L. D., Deuter-Reinhard, M., Doyle, L. V., Wang, J. X., Panganiban-Lustan, L., Stratton, J. R., Apell, G., Winter, J. A., Doyle, M. V., Rosenberg, S., and Kavanaugh, W. M. (1999). Semirational design of a potent, artificial agonist of fibroblast growth factor receptors. Nat Biotechnol 17, 1199-1204.
19. Bame, K. J., Venkatesan, I., Dehdashti, J., McFarlane, J., and Burfeind, R. (2002). Characterization of a novel intracellular heparanase that has a FERM domain. Biochem J 364, 265-274.
20. Batra, R. S., and Kelley, L. C. (2002). Predictors of extensive subclinical spread in nonmelanoma skin cancer treated with Mohs micrographic surgery. Arch Dermatol 138, 1043-1051.
21. Bayatti, N., and Engele, J. (2001). Cyclic AMP modulates the response of central nervous system glia to fibroblast growth factor-2 by redirecting signalling pathways. J Neurochem 78, 972-980.
22. Bazill, G. W., and Dexter, T. M. (1990). Role of endocytosis in the action of ether lipids on WEHI-3B, HL60, and FDCP-mix A4 cells. Cancer Res 50, 7505-7512.
23. Beauvais, D. M., and Rapraeger, A. C. (2003). Syndecan-1-mediated cell spreading requires signaling by alphavbeta3 integrins in human breast carcinoma cells. Exp Cell Res 286, 219-232.
24. Beauvais, D. M., and Rapraeger, A. C. (2004). Syndecans in tumor cell adhesion and signaling. Reprod Biol Endocrinol 2, 3-14.
25. Beck, P. L., and Podolsky, D. K. (1999). Growth factors in inflammatory bowel disease. Inflamm Bowel Dis 5, 44-60.
26. Belting, M., Havsmark, B., Jonsson, M., Persson, S., and Fransson, L. A. (1996). Heparan sulphate/heparin glycosaminoglycans with strong affinity for the growth-promoter spermine have high antiproliferative activity. Glycobiology 6, 121-129.
27. Belting, M., Borsig, L., Fuster, M. M., Brown, J. R., Persson, L., Fransson, L. A., and Esko, J. D. (2002). Tumor attenuation by combined heparan sulfate and polyamine depletion. Proc Natl Acad Sci USA 99, 371-376.
28. Bernard, B. A., Newton, S. A., and Olden, K. (1983). Effect of size and location of the oligosaccharide chain on protease degradation of bovine pancreatic ribonuclease. J Biol Chem 258, 12198-12202.
29. Bernfield, M., Gotte, M., Park, P. W., Reizes, O., Fitzgerald, M. L., Lincecum, J., and Zako, M. (1999). Functions of cell surface heparan sulfate proteoglycans. Annu Rev Biochem 68, 729-777.
30. Berry, D., Kwan, C. P., Shriver, Z., Venkataraman, G., and Sasisekharan, R. (2001). Distinct heparan sulfate glycosaminoglycans are responsible for mediating Fibroblast Growth Factor-2 biological activity though different Fibroblast Growth Factor Receptors. Faseb J 15, 1422-1424,
31. Berry, D., Shriver, Z., Natke, B., Kwan, C. P., Venkataraman, G., and Sasisekharan, R. (2003). Heparan sulphate glycosaminoglycans derived from endothelial cells and smooth muscle cells differentially modulate fibroblast growth factor-2 biological activity through fibroblast growth factor receptor-1. Biochem J 373, 241-249.
32. Berry, D., Lynn, D. M., Sasisekharan, R., and Langer, R. (2004). Poly(beta-amino ester)s promote cellular uptake of heparin and cancer cell death. Chem Biol 11, 487-498.
33. Berry, D., Shriver, Z., Venkataraman, G., and Sasisekharan, R. (2004). Quantitative assessment of FGF regulation by cell surface heparan sulfates. Biochem Biophys Res Commun 314, 994-1000.
34. Berry, D., Jenniskens, G., Dull, R., and Sasisekharan, R. (2003). Functional activity of cell-surface heparan-sulfate glycosaminoglycan is dependent on focal sequence.
35. Berryman, D. E., and Bensadoun, A. (1995). Heparan sulfate proteoglycans are primarily responsible for the maintenance of enzyme activity, binding, and degradation of lipoprotein lipase in Chinese hamster ovary cells. J Biol Chem 270, 24525-24531.
36. Billottet, C., Janji, B., Thiery, J. P., and Jouanneau, J. (2002). Rapid tumor development and potent vascularization are independent events in carcinoma producing FGF-1 or FGF-2. Oncogene 21, 8128-8139.
37. Bjornsson, S. (1998). Quantitation of proteoglycans as glycosaminoglycans in biological fluids using an Alican blue dot analysis. Anal Biochem 256, 229-237.
38. Blackhall, F. H., Merry, C. L., Davies, E. J., and Jayson, G. C. (2001). Heparan sulfate proteoglycans and cancer. Br J Cancer 85, 1094-1098.
39. Blumberg, R. S., and Strober, W. (2001). Prospects for research in inflammatory bowel disease. Jama 285, 643-647.
40. Boehm, T., Folkman, J., Browder, T., and O'Reilly, M. S. (1997). Antiangiogenic therapy of experimental cancer does not induce acquired drug resistance. Nature 390, 404-407.
41. Bogousslavsky, J., Victor, S. J., Salinas, E. O., Pallay, A., Donnan, G. A., Fieschi, C., Kaste, M., Orgogozo, J. M., Chamorro, A., and Desmet, A. (2002). Fiblast (trafermin) in acute stroke: results of the European-Australian phase II/III safety and efficacy trial. Cerebrovasc Dis 14, 239-251.
42. Bonneh-Barkay, D., Shlissel, M., Berman, B., Shaoul, E., Admon, A., Vlodavsky, I., Carey, D. J., Asundi, V. K., Reich-Slotky, R., and Ron, D. (1997). Identification of glypican as a dual modulator of the biological activity of fibroblast growth factors. J Biol Chem 272, 12415-12421.
43. Borregaard, N., and Cowland, J. B. (1997). Granules of the human neutrophilic polymorphonuclear leukocyte. Blood 89, 3503-3521.
44. Borsig, L., Wong, R., Feramisco, J., Nadeau, D. R., Varki, N. M., and Varki, A. (2001). Heparin and cancer revisited: mechanistic connections involving platelets, P-selectin, carcinoma mucins, and tumor metastasis. Proc Natl Acad Sci USA 98, 3352-3357.
45. Bossennec, V., Petitou, M., and Perly, B. (1990). 1H-n.m.r. investigation of naturally occurring and chemically oversulphated dermatan sulphates. Identification of minor monosaccharide residues. Biochem J 267, 625-630.
46. Bottaro, D. P. (2002). The role of extracellular matrix heparan sulfate glycosaminoglycan in the activation of growth factor signaling pathways. Ann N Y Acad Sci 961, 158.
47. Bourin, M. C., Lundgren-Akerlund, E., and Lindahl, U. (1990). Isolation and characterization of the glycosaminoglycan component of rabbit thrombomodulin proteoglycan. J Biol Chem 265, 15424-15431.
48. Bourin, M. C., and Lindahl, U. (1993). Glycosaminoglycans and the regulation of blood coagulation. Biochem J 289 (Pt 2), 313-330.
49. Bousvaros, A., Zurkowski, D., Fishman, S. J., Keough, K., Law, T., Sun, C., and Leichtner, A. M. (1997). Serum basic fibroblast growth factor in pediatric Crohn's disease. Implications for wound healing. Dig Dis Sci 42, 378-386.
50. Brauchle, M., Madlener, M., Wagner, A. D., Angermeyer, K., Lauer, U., Hofschneider, P. H., Gregor, M., and Werner, S. (1996). Keratinocyte growth factor is highly overexpressed in inflammatory bowel disease. Am J Pathol 149, 521-529.
51. Brazeau, G. A., Attia, S., Poxon, S., and Hughes, J. A. (1998). In vitro myotoxicity of selected cationic macromolecules used in non-viral gene delivery. Pharm Res 15, 680-684.
52. Brodsky, R. A., Mukhina, G. L., Li, S., Nelson, K. L., Chiurazzi, P. L., Buckley, J. T., and Borowitz, M. J. (2000). Improved detection and characterization of paroxysmal nocturnal hemoglobinuria using fluorescent aerolysin. Am J Clin Pathol 114, 459-466.
53. Bryckaert, M., Guillonneau, X., Hecquet, C., Perani, P., Courtois, Y., and Mascarelli, F. (2000). Regulation of proliferation-survival decisions is controlled by FGF1 secretion in retinal pigmented epithelial cells. Oncogene 19, 4917-4929.
54. Bulpitt, P., and Aeschlimann, D. (1999). New strategy for chemical modification of hyaluronic acid: preparation of functionalized derivatives and their use in the formation of novel biocompatible hydrogels. J Biomed Mater Res 47, 152-169.
55. Byrne, F. R., Farrell, C. L., Aranda, R., Rex, K. L., Scully, S., Brown, H. L., Flores, S. A., Gu, L. H., Danilenko, D. M., Lacey, D. L., Ziegler, T. R., and Senaldi, G. (2002). rHuKGF ameliorates symptoms in DSS and CD4(+)CD45RB(Hi) T cell transfer mouse models of inflammatory bowel disease. Am J Physiol Gastrointest Liver Physiol 282, G690-701.
56. Cadigan, K. M., and Nusse, R. (1997). Wnt signaling: a common theme in animal development. Genes Dev 11, 3286-3305.
57. Capila, I., and Linhardt, R. J. (2002). Heparin-protein interactions. Angew Chem Int Ed Engl 41, 391-412.
58. Cardin, A. D., and Weintraub, H. J. (1989). Molecular modeling of protein-glycosaminoglycan interactions. Arteriosclerosis 9, 21-32.
59. Carey, D. J., Stahl, R. C., Cizmeci-Smith, G., and Asundi, V. K. (1994). Syndecan-1 expressed in Schwann cells causes morphological transformation and cytoskeletal reorganization and associates with actin during cell spreading. J Cell Biol 124, 161-170.
60. Carey, D. J. (1997). Syndecans: multifunctional cell-surface co-receptors. Biochem J 327 (Pt 1), 1-16.
61. Casu, B., and Lindahl, U. (2001). Structure and biological interactions of heparin and heparan sulfate. Adv Carbohydr Chem Biochem 57, 159-206.
62. Casu, B., Guerrini, M., Naggi, A., Perez, M., Torri, G., Ribatti, D., Carminati, P., Giannini, G., Penco, S., Pisano, C., Belleri, M., Rusnati, M., and Presta, M. (2002). Short heparin sequences spaced by glycol-split uronate residues are antagonists of fibroblast growth factor 2 and angiogenesis inhibitors. Biochemistry 41, 10519-10528.
63. Chang, Z., Meyer, K., Rapraeger, A. C., and Friedl, A. (2000). Differential ability of heparan sulfate proteoglycans to assemble the fibroblast growth factor receptor complex in situ. FASEB J 14, 137-144.
64. Chaouchi, N., Arvanitakis, L., Auffredou, M. T., Blanchard, D. A., Vazquez, A., and Sharma, S. (1995). Characterization of transforming growth factor-beta 1 induced apoptosis in normal human B cells and lymphoma B cell lines. Oncogene 11, 1615-1622.
65. Chauhan-Patel, R., and Spruce, A. E. (1998). Differential regulation of potassium currents by FGF-1 and FGF-2 in embryonic Xenopus laevis myocytes. J Physiol 512 (Pt 1), 109-118.
66. Chellaiah, A. T., McEwen, D. G., Werner, S., Xu, J., and Ornitz, D. M. (1994). Fibroblast growth factor receptor (FGFR) 3. Alternative splicing in immunoglobulin-like domain III creates a receptor highly specific for acidic FGF/FGF-1. J Biol Chem 269, 11620-11627.
67. Chen, G., Ito, Y., Imanishi, Y., Magnani, A., Lamponi, S., and Barbucci, R. (1997). Photoimmobilization of sulfated hyaluronic acid for antithrombogenicity. Bioconjug Chem 8, 730-734.
68. Chen, Y., Chou, K., Fuchs, E., Havran, W. L., and Boismenu, R. (2002). Protection of the intestinal mucosa by intraepithelial gamma delta T cells. Proc Natl Acad Sci USA 99, 14338-14343.
69. Cheng, C. W., Smith, S. K., and Charnock-Jones, D. S. (2003). Wnt-1 signaling inhibits human umbilical vein endothelial cell proliferation and alters cell morphology. Exp Cell Res 291, 415-425.
70. Choubey, D., and Gutterman, J. U. (1997). Inhibition of E2F-4/DP-1-stimulated transcription by p202. Oncogene 15, 291-301.
71. Chu, C. L., Buczek-Thomas, J. A., and Nugent, M. A. (2004). Heparan sulphate proteoglycans modulate fibroblast growth factor-2 binding through a lipid raft-mediated mechanism. Biochem J 379, 331-341.
72. Chua, C. C., Rahimi, N., Forsten-Williams, K., and Nugent, M. A. (2004). Heparan sulfate proteoglycans function as receptors for fibroblast growth factor-2 activation of extracellular signal-regulated kinases 1 and 2. Circ Res 94, 316-323.
73. Ciruna, B., and Rossant, J. (2001). FGF signaling regulates mesoderm cell fate specification and morphogenetic movement at the primitive streak. Dev Cell 1, 37-49.
74. Citores, L., Wesche, J., Kolpakova, E., and Olsnes, S. (1999). Uptake and intracellular transport of acidic fibroblast growth factor: evidence for free and cytoskeleton-anchored fibroblast growth factor receptors. Mol Biol Cell 10, 3835-3848.
75. Clasper, S., Vekemans, S., Fiore, M., Plebanski, M., Wordsworth, P., David, G., and Jackson, D. G. (1999). Inducible expression of the cell surface heparan sulfate proteoglycan syndecan-2 (fibroglycan) on human activated macrophages can regulate fibroblast growth factor action. J Biol Chem 274, 24113-24123.
76. Cobel-Geard, R. J., and Hassouna, H. I. (1983). Interaction of protamine sulfate with thrombin. Am J Hematol 14, 227-233.
77. Conrad, H. E. (1998). Heparin-Binding Proteins (San Diego: Academic Press).
78. Cook, J., and Zitelli, J. A. (1998). Mohs micrographic surgery: a cost analysis. J Am Acad Dermatol 39, 698-703.
79. Cosgrove, R. H., Zacharski, L. R., Racine, E., and Andersen, J. C. (2002). Improved cancer mortality with low-molecular-weight heparin treatment: a review of the evidence. Semin Thromb Hemost 28, 79-87.
80. Couchman, J. R., and Woods, A. (1999). Syndecan-4 and integrins: combinatorial signaling in cell adhesion. J Cell Sci 112 (Pt 20), 3415-3420.
81. Couchman, J. R. (2003). Syndecans: proteoglycan regulators of cell-surface microdomains? Nat Rev Mol Cell Biol 4, 926-937.
82. Cronauer, M. V., Hittmair, A., Eder, I. E., Hobisch, A., Culig, Z., Ramoner, R., Zhang, J., Bartsch, G., Reissigl, A., Radmayr, C., Thurnher, M., and Klocker, H. (1997). Basic fibroblast growth factor levels in cancer cells and in sera of patients suffering from proliferative disorders of the prostate. Prostate 31, 223-233.
83. Crystal, R. G. (1995). Transfer of genes to humans: early lessons and obstacles to success. Science 270, 404-410.
84. Curry, F. E., and Michel, C. C. (1980). A fiber matrix model of capillary permeability. Microvasc Res 20, 96-99.
85. Dai, L., Zientek, P., St. Johns, H., Pasic, P., Chatelier, R., and Griesser, H. J. (1996). Surface modification of polymeric biomaterials. In Surface modification of polymer biomaterials, B. Rastner and D. Castner, eds. (New York: Plenum Press), p. 147.
86. David, G. (1993). Integral membrane heparan sulfate proteoglycans. Faseb J 7, 1023-1030.
87. Davis, J. C., Venkataraman, D., Shriver, Z., Raj, P. A., and Sasisekharan, R. (1999). Oligomeric self-association of basic fibroblast growth factor in the absence of heparin-like glycosaminoglycans. Biochemistry Journal 341, 613-620.
88. Day, R. M., Mitchell, T. J., Knight, S. C., and Forbes, A. (2003). Regulation of epithelial syndecan-1 expression by inflammatory cytokines. Cytokine 21, 224-233.
89. Deguchi, Y., Okutsu, H., Okura, T., Yamada, S., Kimura, R., Yuge, T., Furukawa, A., Morimoto, K., Tachikawa, M., Ohtsuki, S., Hosoya, K., and Terasaki, T. (2002). Internalization of basic fibroblast growth factor at the mouse blood-brain barrier involves a heparan sulfate proteoglycan. J Neurochem 83, 381-389.
90. Delehedde, M., Lyon, M., Sergeant, N., Rahmoune, H., and Fernig, D. G. (2001). Proteoglycans: pericellular and cell surface multireceptors that integrate external stimuli in the mammary gland. J Mammary Gland Biol Neoplasia 6, 253-273.
91. Di Sabatino, A., Ciccocioppo, R., Armellini, E., Morera, R., Ricevuti, L., Cazzola, P., Fulle, I., and Corazza, G. R. (2004). Serum bFGF and VEGF correlate respectively with bowel wall thickness and intramural blood flow in Crohn's disease. Inflamm Bowel Dis 10, 573-577.
92. DiGabriele, A. D., Lax, I., Chen, D. I., Svahn, C. M., Jaye, M., Schlessinger, J., and Hendrickson, W. A. (1998). Structure of a heparin-linked biologically active dimer of fibroblast growth factor. Nature 393, 812-817.
93. Dignass, A. U., Tsunekawa, S., and Podolsky, D. K. (1994). Fibroblast growth factors modulate intestinal epithelial cell growth and migration. Gastroenterology 106, 1254-1262.
94. Dorkin, T. J., Robinson, M. C., Marsh, C., Bjartell, A., Neal, D. E., and Leung, H. Y. (1999). FGF8 over-expression in prostate cancer is associated with decreased patient survival and persists in androgen independent disease. Oncogene 18, 2755-2761.
95. Dorkin, T. J., Robinson, M. C., Marsh, C., Neal, D. E., and Leung, H. Y. (1999). aFGF immunoreactivity in prostate cancer and its co-localization with bFGF and FGF8. J Pathol 189, 564-569.
96. Dudas, J., Ramadori, G., Knittel, T., Neubauer, K., Raddatz, D., Egedy, K., and Kovalszky, I. (2000). Effect of heparin and liver heparan sulphate on interaction of HepG2-derived transcription factors and their cis-acting elements: altered potential of hepatocellular carcinoma heparan sulphate. Biochem J 350 Pt 1, 245-251.
97. Dudek, S. M., and Garcia, J. G. (2001). Cytoskeletal regulation of pulmonary vascular permeability. J Appl Physiol 91, 1487-1500.
98. Dull, R. O., Dinavahi, R., Schwartz, L., Humphries, D. E., Berry, D., Sasisekharan, R., and Garcia, J. G. (2003). Lung endothelial heparan sulfates mediate cationic peptide-induced barrier dysfunction: a new role for the glycocalyx. Am J Physiol Lung Cell Mol Physiol 285, L986-995.
99. Dull, R. O., Dinavahi, R., Schwartz, L., Humphries, D. E., Berry, D., Sasisekharan, R., and Garcia, J. G. N. (2003). Lung endothelial heparan sulfates mediate cationic peptide-Induced barrier dysfunction: a new role for the glycocalyx. Am J Physiol Lung Cell Mol. Physiol.
100. Duteil, S., Gariel, P., Girault, S., Mallet, A., Feve, C., and Siret, L. (1999). Identification of heparin oligosaccharides by direct coupling of capillary electrophoresis/ionspray-mass spectrometry. Rapid Commun Mass Spectrom 13, 1889-1898.
101. Edelman, E. R., Adams, D. H., Karnovsky, M. J. (1990). Effect of controlled adventitial heparin delivery on smooth muscle cell proliferation following endothelial injury. Proc Natl Acad Sci USA 87, 3773-3777.
102. Edovitsky, E., Elkin, M., Zcharia, E., Peretz, T., and Vlodavsky, I. (2004). Heparanase gene silencing, tumor invasiveness, angiogenesis, and metastasis. J Natl Cancer Inst 96, 1219-1230.
103. el-Hariry, I., Pagnatelli, M., and Lemoine, N. (1997). Fibroblast growth factor 1 and fibroblast growth factor 2 immunoreactivity in gastrointestinal tumours. J Pathol 181, 39-45.
104. Ernst, S., Langer, R., Cooney, C. L., and Sasisekharan, R. (1995). Enzymatic degradation of glycosaminoglycans. Crit Rev in Biochem Mol Biol 30, 387-444.
105. Ernst, S., Venkataraman, G., Winkler, S., Godavarti, R., Langer, R., Cooney, C. L., and Sasisekharan, R. (1996). Expression in Escherichia coli, purification and characterization of heparinase I from Flavobacterium heparinum. Biochem J 315 (Pt 2), 589-597.
106. Ernst, S., et al (1996). Expression in Escherichia coli, purification and characterization of heparinase I from Flavobacterium heparinum. Biochemical Journal 315, 589-597.
107. Eroglu, A., Toner, M., and Toth, T. L. (2002). Beneficial effect of microinjected trehalose on the cryosurvival of human oocytes. Fertil Steril 77, 152-158.
108. Esko, J. D., and Lindahl, U. (2001). Molecular diversity of heparan sulfate. J Clin Invest 108, 169-173.
109. Esko, J. D., and Selleck, S. B. (2002). Order out of chaos: assembly of ligand binding sites in heparan sulfate. Annu Rev Biochem 71, 435-471.
110. Ettenson, D. S., Koo, E. W. Y., Januzzi, J. L., and Edelman, E. R. (2000). Endothelial heparan sulfate is necessary but not sufficient for control of vascular smooth muscle cell growth. J Cell Physiol 184, 93-100.
111. Faham, S., Hileman, R. E., Fromm, J. R., Linhardt, R. J., and Rees, D. C. (1996). Heparin structure and interactions with basic fibroblast growth factor. Science 271, 1116-1120.
112. Faham, S., Hileman, R. E., Fromm, J. R>, Lindhart, R. J., and Rees, D. C. (1996). Heparin structure and interactions with basic fibroblast growth factor. Science 271, 1116-1120.
113. Fairbrother, W. J., Champe, M. A., Christinger, H. W., Keyt, B. A., and Starovasnik, M. A. (1998). Solution structure of the heparin-binding domain of vascular endothelial growth factor. Structure 6, 637-648.
114. Fannon, M., and Nugent, M. A. (1996). Basic fibroblast growth factor binds its receptors, is internalized, and stimulates DNA synthesis in Balb/c3T3 cells in the absence of heparan sulfate. J Biol Chem 271, 17949-17956.
115. Fannon, M., Forsten, K. E., and Nugent, M. A. (2000). Potentiation and inhibition of bFGF binding to heparin: a model for regulation of cellular response. Biochemistry 39, 1434-1445.
116. Fannon, M., Forsten-Williams, K., Dowd, C. J., Freedman, D. A., Folkman, J., and Nugent, M. A. (2003). Binding inhibition of angiogenic factors by heparan sulfate proteoglycans in aqueous humor: potential mechanism for maintenance of an avascular environment. Faseb J 17, 902-904.
117. Farquhar, M. G., and Palade, G. E. (1963). Junctional complexes in various epithelia. J Cell Biol 17, 375-412.
118. Feng, S., Wang, F., Matsubara, A., Kan, M., and McKeehan, W. L. (1997). Fibroblast growth factor receptor 2 limits and receptor 1 accelerates tumorigenicity of prostate epithelial cells. Cancer Res 57, 5369-5378.
119. Fernig, D. G., and Gallagher, J. T. (1994). Fibroblast growth factors and their receptors: an information network controlling tissue growth, morphogenesis and repair. Prog Growth Factor Res 5, 353-377.
120. Ferrara, N., Gerber, H. P., and LeCouter, J. (2003). The biology of VEGF and its receptors. Nat Med 9, 669-676.
121. Fialka, I., Steinlein, P., Ahorn, H., Bock, G., Burbelo, P. D., Haberfellner, M., Lottspeich, F., Paiha, K., Pasquali, C., and Huber, L. A. (1999). Identification of syntenin as a protein of the apical early endocytic compartment in Madin-Darby canine kidney cells. J Biol Chem 274, 26233-26239.
122. Fidler, I. J., and Kripke, M. L. (1977). Metastasis results from preexisting variant cells within a malignant tumor. Science 197, 893-895.
123. Filmus, J., and Selleck, S. B. (2001). Glypicans: proteoglycans with a surprise. J Clin Invest 108, 497-501.
124. Finch, P. W., Rubin, J. S., Miki, T., Ron, D., and Aaronson, S. A. (1989). Human KGF is FGF-related with properties of a paracrine effector of epithelial cell growth. Science 245, 752-755.
125. Finch, P. W., Pricolo, V., Wu, A., and Finkelstein, S. D. (1996). Increased expression of keratinocyte growth factor messenger RNA associated with inflammatory bowel disease. Gastroenterology 110, 441-451.
126. Finch, P. W., and Cheng, A. L. (1999). Analysis of the cellular basis of keratinocyte growth factor overexpression in inflammatory bowel disease. Gut 45, 848-855.
127. Fisher, M., Meadows, M. E., Do, T., Weise, J., Trubestskoy, V., Charette, M., and Finklestein, S. P. (1995). Delayed treatment with intravenous basic fibroblast growth factor reduces infarct size following permanent focal cerebralischemia in rats. J Cereb Blood Flow Metab 15, 953-959.
128. Fitzgerald, M. L., Wang, Z., Park, P. W., Murphy, G., and Bernfield, M. (2000). Shedding of syndecan-1 and -4 ectodomains is regulated by multiple signaling pathways and mediated by a TIMP-3-sensitive metalloproteinase. J Cell Biol 148, 811-824.
129. Fitzgerald, M. L., Wang, Z., Park, P. W., Murphy, G., and Bernfield, M. (2000). Shedding of syndecan-1 and -4 ectodomains is regulated by multiple signaling pathways and mediated by a TIMP-3-sensitive metalloproteinase. J Cell Biol 148, 811-824.
130. Florian, J. A., Kosky, J. R., Ainslie, K., Pang, Z., Dull, R. O., and Tarbell, J. M. (2003). Heparan sulfate proteoglycan is a mechanosensor on endothelial cells. Circ Res 93, e136-142.
131. Folkman, J., Langer, R., Linhardt, R. J., Haudenschild, C., and Taylor, S. (1983). Angiogenesis inhibition and tumor regression caused by heparin or a heparin fragment in the presence of cortisone. Science 221, 719-725.
132. Folkman, J., Szabo, S., Stovroff, M., McNeil, P., Li, W., and Shing, Y. (1991). Duodenal ulcer. Discovery of a new mechanism and development of angiogenic therapy that accelerates healing. Ann Surg 214, 414-425; discussion 426-417.
133. Folkman, J. (2001). Angiogenesis-dependent diseases. Semin Oncol 28, 536-542.
134. Forsten, K. E., Courant, N. A., and Nugent, M. A. (1997). Endothelial proteoglycans inhibit bFGF binding and mitogenesis. J Cell Physiol 172, 209-220.
135. Friedmann, Y., Vlodavsky, I., Aingorn, H., Aviv, A., Peretz, T., Pecker, I., and Pappo, O. (2000). Expression of heparanase in normal, dysplastic, and neoplastic human colonic mucosa and stroma. Evidence for its role in colonic tumorigenesis. Am J Pathol 157, 1167-1175.
136. Fujiwara, Y., and Kaji, T. (1999). Possible mechanism for lead inhibition of vascular endothelial cell proliferation: a lower response to basic fibroblast growth factor through inhibition of heparan sulfate synthesis. Toxicology 133, 147-157.
137. Fukatsu, T., Sobue, M., Nagasaka, T., Ohiwa, N., Fukata, S., Nakashima, N., and Takeuchi, J. (1988). Immunohistochemical localization of chondroitin sulphate and dermatan sulphate proteoglycans in tumour tissues. Br J Cancer 57, 74-78.
138. Gallagher, J. T., Turnbull, J. E., and Lyon, M. (1992). Patterns of sulphation in heparan sulphate: polymorphism based on a common structural theme. Int J Biochem 24, 553-560.
139. Gallagher, J. T., Turnbull, J. E., and Lyon, M. (1992). Heparan sulphate proteoglycans: molecular organisation of membrane-associated species and an approach to polysaccharide sequence analysis. Adv Exp Med Biol 313, 49-57.
140. Gallagher, J. T. (1997). Structure-activity relationship of heparan sulphate. Biochem Soc Trans 25, 1206-1209.
141. Garcia, J. G., Davis, H. W., and Patterson, C. E. (1995). Regulation of endothelial cell gap formation and barrier dysfunction: role of myosin light chain phosphorylation. J Cell Physiol 163, 510-522.
142. Garcia, J. G., Lazar, V., Gilbert-McClain, L. I., Gallagher, P. J., and Verin, A. D. (1997). Myosin light chain kinase in endothelium: molecular cloning and regulation. Am J Respir Cell Mol Biol 16, 489-494.
143. Garcia, J. G., Schaphorst, K. L., Shi, S., Verin, A. D., Hart, C. M., Callahan, K. S., and Patterson, C. E. (1997). Mechanisms of ionomycin-induced endothelial cell barrier dysfunction. Am J Physiol 273, L172-184.
144. Garcia, J. G., Wang, P., Schaphorst, K. L., Becker, P. M., Borbiev, T., Liu, F., Birukova, A., Jacobs, K., Bogatcheva, N., and Verin, A. D. (2002). Critical involvement of p38 MAP kinase in pertussis toxin-induced cytoskeletal reorganization and lung permeability. Faseb J 16, 1064-1076.
145. Gardiner, K. R., Anderson, N. H., Rowlands, B. J., and Barbul, A. (1995). Colitis and colonic mucosal barrier dysfunction. Gut 37, 530-535.
146. Gautam, N., Olofsson, A. M., Herwald, H., Iversen, L. F., Lundgren-Akerlund, E., Hedqvist, P., Arfors, K. E., Flodgaard, H., and Lindbom, L. (2001). Heparin-binding protein (HBP/CAP37): a missing link in neutrophil-evoked alteration of vascular permeability. Nat Med 7, 1123-1127.
147. Gavioli, R., Frisan, T., Vertuani, S., Bornkamm, G. W., and Masucci, M. G. (2001). c-myc overexpression activates alternative pathways for intracellular proteolysis in lymphoma cells. Nat Cell Biol 3, 283-288.
148. Giaever, I., and Keese, C. R. (1993). A morphological biosensor for mammalian cells. Nature 366, 591-592.
149. Gimbrone, M. A. (1995). Vascular endothelium in health and disease. In Molecular Cardiovascular Medicine, E. Haber, ed. (Scientific American Medicine), pp. 49-61.
150. Giraux, J. L., Matou, S., Bros, A., Tapon-Bretaudiere, J., Letourneur, D., Fischer, A. M. (1998). Modulation of human endothelial cell proliferation and migration by fucoidan and heparin. Eur J Cell Biol 77, 352-359.
151. Giri, D., Ropiquet, F., and Ittmann, M. (1999). Alterations in expression of basic fibroblast growth factor (FGF) 2 and its receptor FGFR-1 in human prostate cancer. Clin Cancer Res 5, 1063-1071.
152. Giri, D., Ropiquet, F., and Ittmann, M. (1999). FGF9 is an autocrine and paracrine prostatic growth factor expressed by prostatic stromal cells. J Cell Physiol 180, 53-60.
153. Givol, D., and Yayon, A. (1992). Complexity of FGF receptors: genetic basis for structural diversity and functional specificity. Faseb J 6, 3362-3369.
154. Godavarti, R., Cooney, C. L., Langer, R., and Sasisekharan, R. (1996). Heparinase I from Flavobacterium heparinum. Identification of a critical histidine residue essential for catalysis as probed by chemical modification and site-directed mutagenesis. Biochemistry 35, 6846-6852.
155. Godavarti, R., Davis, M., Venkataraman, G., Cooney, C., Langer, R., and Sasisekharan, R. (1996). Heparinase III from Flavobacterium heparinum: cloning and recombinant expression in Escherichia coli. Biochem Biophys Res Commun 225, 751-758.
156. Godavarti, R., and Sasisekharan, R. (1998). Heparinase I from Flavobacterium heparinum. Role of positive charge in enzymatic activity. J Biol Chem 273, 248-255.
157. Goldshmidt, O., Zcharia, E., Abramovitch, R., Metzger, S., Aingorn, H., Friedmann, Y., Schirrmacher, V., Mitrani, E., and Vlodavsky, I. (2002). Cell surface expression and secretion of heparanase markedly promote tumor angiogenesis and metastasis. Proc Natl Acad Sci USA 99, 10031-10036.
158. Graeven, U., Rodeck, U., Karpinski, S., Jost, M., Philippou, S., and Schmiegel, W. (2001). Modulation of angiogenesis and tumorigenicity of human melanocytic cells by vascular endothelial growth factor and basic fibroblast growth factor. Cancer Res 61, 7282-7290.
159. Gribbon, P., Heng, B. C., and Hardingham, T. E. (1999). The molecular basis of the solution properties of hyaluronan investigated by confocal fluorescence recovery after photobleaching. Biophys J 77, 2210-2216.
160. Grootjans, J. J., Zimmermann, P., Reekmans, G., Smets, A., Degeest, G., Durr, J., and David, G. (1997). Syntenin, a PDZ protein that binds syndecan cytoplasmic domains. Proc Natl Acad Sci USA 94, 13683-13688.
161. Gross, J. L., Morrison, R. S., Eidsvoog, K., Herblin, W. F., Kornblith, P. L., and Dexter, D. L. (1990). Basic fibroblast growth factor: a potential autocrine regulator of human glioma cell growth. J Neurosci Res 27, 689-696.
162. Guerrini, M., Agulles, T., Bisio, A., Hricovini, M., Lay, L., Naggi, A., Poletti, L., Sturiale, L., Torri, G., and Casu, B. (2002). Minimal heparin/heparan sulfate sequences for binding to fibroblast growth factor-1. Biochem Biophys Res Commun 292, 222-230.
163. Guimond, S., Maccarana, M., Olwin, B. B., Lindahl, U., and Rapraeger, A. C. (1993). Activating and inhibitory heparin sequences for FGF-2 (basic FGF). Distinct requirements for FGF-1, FGF-2 and FGF-4. J Biol Chem 268, 23906-23914.
164. Guimond, S. E., Turnbull, J. E (1999). Fibroblast growth factor receptor signalling is dictated by specific heparan sulfate saccharides. Current Biology 9, 1343-1346.
165. Gumbiner, B. M. (1996). Cell adhesion: the molecular basis of tissue architecture and morphogenesis. Cell 84, 345-357.
166. Habuchi, H., Suzuki, S., Saito, T., Tamura, T., Harada, T., Yoshida, K., and Kimata, K. (1992). Structure of a heparan sulphate oligosaccharide that binds to basic fibroblast growth factor. Biochem J 285 (Pt 3), 805-813.
167. Hahnenberger, R., Jakobson, A. M., Ansari, A., Wehler, T., Svahn, C. M., and Lindahl, U. (1993). Low-sulphated oligosaccharides derived from heparan sulphate inhibit normal angiogenesis. Glycobiology 3, 567-573.
168. Halaban, R. (1999). Melanoma cell autonomous growth: the Rb/E2F pathway. Cancer Metastasis Rev 18, 333-343.
169. Han, D. S., Li, F., Holt, L., Connolly, K., Hubert, M., Miceli, R., Okoye, Z., Santiago, G., Windle, K., Wong, E., and Sartor, R. B. (2000). Keratinocyte growth factor-2 (FGF-10) promotes healing of experimental small intestinal ulceration in rats. Am J Physiol Gastrointest Liver Physiol 279, G1011-G1022.
170. Han, R. O., Ettenson, D. S., Koo, E. W., and Edelman, E. R. (1997). Heparin/heparan sulfate chelation inhibits control of vascular repair by tissue-engineering endothelial cells. Am J Physiol 273, H2586-2595.
171. Han, X., Fink, M. P., and Delude, R. L. (2003). Proinflammatory cytokines cause NO*-dependent and -independent changes in expression and localization of tight junction proteins in intestinal epithelial cells. Shock 19, 229-237.
172. Hanahan, D., and Weinberg, R. A. (2000). The hallmarks of cancer. Cell 100, 57-70.
173. Hansen, C. A., Schroering, A. G., Carey, D. J., and Robishaw, J. D. (1994). Localization of a heterotrimeric G protein gamma subunit to focal adhesions and associated stress fibers. J Cell Biol 126, 811-819.
174. Hartley, P. G., McArthur, S. L., McLean, K. M., and Griesser, H. J. (2002). Physiochemical properties of polysaccharide coatings based on gradted multilayer assemblies. Langmuir 18, 2383-2394.
175. Hawkins, C. L., and Davies, M. J. (1998). Degradation of hyaluronic acid, poly- and monosaccharides, and model compounds by hypochlorite: evidence for radical intermediates and fragmentation. Free Radic Biol Med 24, 1396-1410.
176. He, J., and Baum, L. G. (2003). Presentation of galectin-1 by extracellular matrix triggers T cell death. J Biol. Chem.
177. Hermiston, M. L., and Gordon, J. I. (1995). Inflammatory bowel disease and adenomas in mice expressing a dominant negative N-cadherin. Science 270, 1203-1207.
178. Herr, A. B., Ornitz, D. M., Sasisekharan, R., Venkataraman, G., and Waksman, G. (1997). Heparin-induced self-association of fibroblast growth factor-2. Evidence for two oligomerization processes. J Biol Chem 272, 16382-16389.
179. Hildebrandt, P. (2002). Glycosaminoglycans—all round talents in coating technology. Biomed Tech (Berl) 47 Suppl 1 Pt 1, 476-478.
180. Ho, C. L., Sheu, L. F., and Li, C. Y. (2002). Immunohistochemical expression of basic fibroblast growth factor, vascular endothelial growth factor, and their receptors in stage 1V non-Hodgkin lymphoma. Appl Immunohistochem Mol Morphol 10, 316-321.
181. Hopkins, A. M., Walsh, S. V., Verkade, P., Boquet, P., and Nusrat, A. (2003). Constitutive activation of Rho proteins by CNF-1 influences tight junction structure and epithelial barrier function. J Cell Sci 116, 725-742.
182. Horowitz, A., Tkachenko, E., and Simons, M. (2002). Fibroblast growth factor-specific modulation of cellular response by syndecan-4. J Cell Biol 157, 715-725.
183. Hsia, E., Richardson, T. P., and Nugent, M. A. (2003). Nuclear localization of basic fibroblast growth factor is mediated by heparan sulfate proteoglycans through protein kinase C signaling. J Cell Biochem 88, 1214-1225.
184. Hu, X., Adamson, R. H., Liu, B., Curry, F. E., and Weinbaum, S. (2000). Starling forces that oppose filtration after tissue oncotic pressure is increased. Am J Physiol Heart Circ Physiol 279, H1724-1736.
185. Huhtala, M. T., Pentikainen, O. T., and Johnson, M. S. (1999). A dimeric ternary complex of FGFR [correction of FGFR1], heparin and FGF-1 leads to an ‘electrostatic sandwich’ model for heparin binding. Structure Fold Des 7, 699-709.
186. Hulett, M. D., Freeman, C., Hamdorf, B. J., Baker, R. T., Harris, M. J., and Parish, C. R. (1999). Cloning of mammalian heparanase, an important enzyme in tumor invasion and metastasis. Nat Med 5, 803-809.
187. Hull, M. A., Cullen, D. J., Hudson, N., and Hawkey, C. J. (1995). Basic fibroblast growth factor treatment for non-steroidal anti-inflammatory drug associated gastric ulceration. Gut 37, 610-612.
188. Humphries, D. E., Lee, S. L., Fanburg, B. L., and Silbert, J. E. (1986). Effects of hypoxia and hyperoxia on proteoglycan production by bovine pulmonary artery endothelial cells. J Cell Physiol 126, 249-253.
189. Humphries, D. E., Wong, G. W., Friend, D. S., Gurish, M. F., Qiu, W. T., Huang, C., Sharpe, A. H., and Stevens, R. L. (1999). Heparin is essential for the storage of specific granule proteases in mast cells. Nature 400, 769-772.
190. Iba, K., Albrechtsen, R., Gilpin, B., Frohlich, C., Loechel, F., Zolkiewska, A., Ishiguro, K., Kojima, T., Liu, W., Langford, J. K., Sanderson, R. D., Brakebusch, C., Fassler, R., and Wewer, U. M. (2000). The cysteine-rich domain of human ADAM 12 supports cell adhesion through syndecans and triggers signaling events that lead to beta1 integrin-dependent cell spreading. J Cell Biol 149, 1143-1156.
191. Ibrahimi, O. A., Eliseenkova, A. V., Plotnikov, A. N., Yu, K., Ornitz, D. M., and Mohammadi, M. (2001). Structural basis for fibroblast growth factor receptor 2 activation in Apert syndrome. Proc Natl Acad Sci USA 98, 7182-7187.
192. Inman, G. J., and Allday, M. J. (2000). Apoptosis induced by TGF-beta 1 in Burkitt's lymphoma cells is caspase 8 dependent but is death receptor independent. J Immunol 165, 2500-2510.
193. Inman, G. J., Binne, U. K., Parker, G. A., Farrell, P. J., and Allday, M. J. (2001). Activators of the Epstein-Barr virus lytic program concomitantly induce apoptosis, but lytic gene expression protects from cell death. J Virol 75, 2400-2410.
194. Iozzo, R. V., and Murdoch, A. D. (1996). Proteoglycans of the extracellular environment: clues from the gene and protein side offer novel perspectives in molecular diversity and function. Faseb J 10, 598-614.
195. Iozzo, R. V. (1998). Matrix proteoglycans: from molecular design to cellular function. Annu Rev Biochem 67, 609-652.
196. Iozzo, R. V., and San Antonio, J. D. (2001). Heparan sulfate proteoglycans: heavy hitters in the angiogenesis arena. Journal of Clinical Investigation 108, 349-355.
197. Ishihara, M. (1994). Structural requirements in heparin for binding and activation of FGF-1 and FGF-4 are different from that for FGF-2. Glycobiology 4, 817-824.
198. Itano, N., Sawai, T., Atsumi, F., Miyaishi, O., Taniguchi, S., Kannagi, R., Hamaguchi, M., and Kimata, K. (2004). Selective expression and functional characteristics of three mammalian hyaluronan synthases in oncogenic malignant transformation. J Biol Chem 279, 18679-18687.
199. Izzard, C. S., Radinsky, R., and Culp, L. A. (1986). Substratum contacts and cytoskeletal reorganization of BALB/c 3T3 cells on a cell-binding fragment and heparin-binding fragments of plasma fibronectin. Exp Cell Res 165, 320-336.
200. Jacks, T., and Weinberg, R. A. (2002). Taking the study of cancer cell survival to a new dimension. Cell 111, 923-925.
201. Jackson, M. W., Roberts, J. S., Heckford, S. E., Ricciardelli, C., Stahl, J., Choong, C., Horsfall, D. J., and Tilley, W. D. (2002). A potential autocrine role for vascular endothelial growth factor in prostate cancer. Cancer Res 62, 854-859.
202. Jackson, R. L., Busch, S. J., and Cardin, A. D. (1991). Glycosaminoglycans: molecular properties, protein interactions, and role in physiological processes. Physiol Rev 71, 481-539.
203. Jameson, J., Ugarte, K., Chen, N., Yachi, P., Fuchs, E., Boismenu, R., and Havran, W. L. (2002). A role for skin gammadelta T cells in wound repair. Science 296, 747-749.
204. Jandik, K. A., Gu, K., and Linhardt, R. J. (1994). Action pattern of polysaccharide lyases on glycosaminoglycans. Glycobiology 4, 289-296.
205. Jeffers, M., McDonald, W. F., Chillakuru, R. A., Yang, M., Nakase, H., Deegler, L. L., Sylander, E. D., Rittman, R., Bendele, A., Sartor, R. B., and Lichenstein, H. S. (2002). A novel human fibroblast growth factor treats experimental intestinal inflammation. Gastroenterology 123, 1151-1162.
206. Jemal, A., Murray, T., Ward, E., Samuels, A., Tiwari, R. C., Ghafoor, A., Feuer, E. J., and Thun, M. J. (2005). Cancer statistics, 2005. CA Cancer J Clin 55, 10-30.
207. Jin, L., Abrahams, J. P., Skinner, R., Petitou, M., Pike, R. N., and Carrell, R. W. (1997). The anticoagulant activation of antithrombin by heparin. Proceedings of the National Academy of Sciences USA 94, 14683-14688.
208. Jones, T. A., and Schallert, T. (1994). Use-dependent growth of pyramidal neurons after neocortical damage. J Neurosci 14, 2140-2152.
209. Joukov, V., Pajusola, K., Kaipainen, A., Chilov, D., Lahtinen, I., Kukk, E., Saksela, O., Kalkkinen, N., and Alitalo, K. (1996). A novel vascular endothelial growth factor, VEGF-C, is a ligand for the Flt4 (VEGFR-3) and KDR (VEGFR-2) receptor tyrosine kinases. Embo J 15, 290-298.
210. Kabanov, A. V., and Kabanov, V. A. (1995). DNA complexes with polycations for the delivery of genetic material into cells. Bioconjug Chem 6, 7-20.
211. Kalluri, R. (2003). Basement membranes: structure, assembly and role in tumour angiogenesis. Nat Rev Cancer 3, 422-433.
212. Kamp, P., Strathmann, A., and Ragg, H. (2001). Heparin cofactor II, antithrombin-beta and their complexes with thrombin in human tissues. Thromb Res 101, 483-491.
213. Kan, M., Wu, X., Wang, F., and McKeehan, W. L. (1999). Specificity for fibroblast growth factors determined by heparan sulfate in a binary complex with the receptor kinase. J Biol Chem 274, 15947-15952.
214. Kan, M., Wang, F., Xu, J., Crabb, J. W., Hou, J., and McKeehan, W. L. (1993). An essential heparin-binding domain in the fibroblast growth factor receptor kinase. Science 259, 1918-1921.
215. Kanai, M., Rosenberg, I., and Podolsky, D. K. (1997). Cytokine regulation of fibroblast growth factor receptor 3 IIIb in intestinal epithelial cells. Am J Physiol 272, G885-G893.
216. Kanazawa, S., Tsunoda, T., Onuma, E., Majima, T., Kagiyama, M., and Kikuchi, K. (2001). VEGF, basic-FGF, and TGF-beta in Crohn's disease and ulcerative colitis: a novel mechanism of chronic intestinal inflammation. Am J Gastroenterol 96, 822-828.
217. Kannan, K., Sharpless, N. E., Xu, J., O'Hagan, R. C., Bosenberg, M., and Chin, L. (2003). Components of the Rb pathway are critical targets of UV mutagenesis in a murine melanoma model. Proc Natl Acad Sci USA 100, 1221-1225.
218. Kaslovsky, R. A., Horgan, M. J., Lum, H., McCandless, B. K., Gilboa, N., Wright, S. D., and Malik, A. B. (1990). Pulmonary edema induced by phagocytosing neutrophils. Protective effect of monoclonal antibody against phagocyte CD18 integrin. Circ Res 67, 795-802.
219. Kato, M., Wang, H., Kainulainen, V., Fitzgerald, M. L., Ledbetter, S., Ornitz, D. M., and Bernfield, M. (1998). Physiological degradation converts the soluble syndecan-1 ectodomain from an inhibitor to a potent activator of FGF-2. Nat Med 4, 691-697.
220. Katoh, M. (2001). Differential regulation of WNT2 and WNT2B expression in human cancer. Int J Mol Med 8, 657-660.
221. Katoh, M. (2002). Regulation of WNT3 and WNT3A mRNAs in human cancer cell lines NT2, MCF-7, and MKN45. Int J Oncol 20, 373-377.
222. Kawakami, N., Kashiwagi, S., Kitahara, T., Yamashita, T., and Ito, H. (1995). Effect of local administration of basic fibroblast growth factor against neuronal damage caused by transient intracerebral mass lesion in rats. Brain Res 697, 104-111.
223. Kawamata, T., Alexis, N. E., Dietrich, W. D., and Finkletsin, S. P. (1996). Intracisternal basic fibroblast growth factor (bFGF) enhances behavioral recovery following focal cerebral infarction in the rat. J Cereb Blood Flow Metab 16, 542-547.
224. Kawamata, T., Dietrich, W. D., Schallert, T., Gotts, J. E., Cocke, R. R., Benowitz, L. I., and Finklestein, S. P. (1997). Intracisternal basic fibroblast growth factor enhances functional recovery and up-regulates the expression of a molecular marker of neuronal sprouting. Proc Natl Acad Sci USA 94, 8179-8184.
225. Kawamata, T., Ren, J., Cha, J. H., and Finklestein, S. P. (1999). Intracisternal antisense oligonucleotide to growth associated protein-43 blocks the recovery-promoting effects of basic fibroblast growth factor after focal stroke. Exp Neurol 158, 89-96.
226. Kawashima, H., Atarashi, K., Hirose, M., Hirose, J., Yamada, S., Sugahara, K., and Miyasaka, M. (2002). Oversulfated chondroitin/dermatan sulfates containing GlcAbeta1/fdoAalpha1-3GalNAc(4,6-O-disulfate) interact with L- and P-selectin and chemokines. J Biol Chem 277, 12921-12930.
227. Keiser, N., Venkataraman, G., Shriver, Z., and Sasisekharan, R. (2001). Direct isolation and sequencing of specific protein-binding glycosaminoglycans. Nat Med 7, 123-128.
228. Keuren, J. F., Wielders, S. J., Willems, G. M., Morra, M., Cahalan, L., Cahalan, P., and Lindhout, T. (2003). Thrombogenicity of polysaccharide-coated surfaces. Biomaterials 24, 1917-1924.
229. Khan, M. Y., Jaikaria, N. S., Frenz, D. A., Villanueva, G., and Newman, S. A. (1988). Structural changes in the NH2-terminal domain of fibronectin upon interaction with heparin. Relationship to matrix-driven translocation. J Biol Chem 263, 11314-11318.
230. Khan, S. R., and Kok, D. J. (2004). Modulators of urinary stone formation. Front Biosci 9, 1450-1482.
231. Kim, H. R., Wheeler, M. A., Wilson, C. M., Iida, J., Eng, D., Simpson, M. A., McCarthy, J. B., and Bullard, K. M. (2004). Hyaluronan facilitates invasion of colon carcinoma cells in vitro via interaction with CD44. Cancer Res 64, 4569-4576.
232. Kitagawa, H., Tanaka, Y., Yamada, S., Seno, N., Haslam, S. M., Morris, H. R., Dell, A., and Sugahara, K. (1997). A novel pentasaccharide sequence GlcA(3-sulfate)(beta1-3)GalNAc(4-sulfate)(beta1-4)(Fuc alpha1-3)GlcA(beta1-3)GalNAc(4-sulfate) in the oligosaccharides isolated from king crab cartilage chondroitin sulfate K and its differential susceptibility to chondroitinases and hyaluronidase. Biochemistry 36, 3998-4008.
233. Klagsbrun, M., and Edelman, E. R. (1989). Biological and biochemical properties of fibroblast growth factors. Implications for the pathogenesis of atherosclerosis. Arteriosclerosis 9, 269-278.
234. Klagsbrun, M., and Baird, A. (1991). A dual receptor system is required for basic fibroblast growth factor activity. Cell 67, 229-231.
235. Klein, E., Klein, G., Nadkarni, J. S., Nadkarni, J. J., Wigzell, H., and Clifford, P. (1968). Surface IgM-kappa specificity on a Burkitt lymphoma cell in vivo and in derived culture lines. Cancer Res 28, 1300-1310.
236. Klein, G. (1981). The role of gene dosage and genetic transpositions in carcinogenesis. Nature 294, 313-318.
237. Klein, G. (1983). Specific chromosomal translocations and the genesis of B-cell-derived tumors in mice and men. Cell 32, 311-315.
238. Klein, M. D., Drongowski, R. A., Linhardt, R. J., and Langer, R. S. (1982). A colorimetric assay for chemical heparin in plasma. Anal Biochem 124, 59-64.
239. Knutson, J. R., Iida, J., Fields, G. B., and McCarthy, J. B. (1996). CD44/chondroitin sulfate proteoglycan and alpha 2 beta 1 integrin mediate human melanoma cell migration on type IV collagen and invasion of basement membranes. Mol Biol Cell 7, 383-396.
240. Kobayashi, Y., Okamoto, A., and Nishinari, K. (1994). Viscoelasticity of hyaluronic-acid with different molecular-weights. Biorheology 31, 234-244.
241. Koketsu, N., Berlove, D. J., Moskowitz, M. A., Kowall, N. W., Caday, C. G., and Finklestein, S. P. (1994). Pretreatment with intraventricular basic fibroblast growth factor decreases infarct size following focal cerebral ischemia in rats. Ann Neurol 35, 451-457.
242. Koliopanos, A., Friess, H., Kleeff, J., Shi, X., Liao, Q., Pecker, I., Vlodavsky, I., Zimmermann, A., and Buchler, M. W. (2001). Heparanase expression in primary and metastatic pancreatic cancer. Cancer Res 61, 4655-4659.
243. Kolset, S. O., Prydz, K., and Pejler, G. (2004). Intracellular proteoglycans. Biochem J 379, 217-227.
244. Konduri, S., Lakka, S. S., Tasiou, A., Yanamandra, N., Gondi, C. S., Dinh, D. H., Olivero, W. C., Gujrati, M., and Rao, J. S. (2001). Elevated levels of cathepsin B in human glioblastoma cell lines. Int J Oncol 19, 519-524.
245. Kresse, H., and Schonherr, E. (2001). Proteoglycans of the extracellular matrix and growth control. J Cell Physiol 189, 266-274.
246. Kreuger, J., Salmivirta, M., Sturiale, L., Giminez-Gallego, G., and Lindhahl, U. (2001). Sequence analysis of heparan sulfate epitopes with graded affinities for fibroblast growth factors 1 and 2. J Biol Chem 276, 30744-30752.
247. Kreuger, J., Matsumoto, T., Vanwildemeersch, M., Sasaki, T., Timpl, R., Claesson-Welsh, L., Spillmann, D., and Lindahl, U. (2002). Role of heparan sulfate domain organization in endostatin inhibition of endothelial cell function. Embo J 21, 6303-6311.
248. Krpski, W. C., Bass, A., Kelly, A. B., Marzec, U. M., Hanson, S. R., and Harker, L. A. (1990). Heparin-resistant thrombus formation by endovascular stents in baboons. Interruption by a synthetic antithrombin. Circulation 82, 570-577.
249. Kubo, H., Cao, R., Brakenhielm, E., Makinen, T., Cao, Y., and Alitalo, K. (2002). Blockade of vascular endothelial growth factor receptor-3 signaling inhibits fibroblast growth factor-2-induced lymphangiogenesis in mouse cornea. Proc Natl Acad Sci USA 99, 8868-8873.
250. Kwan, C.-P., Venkataraman, G., Shriver, Z., Raman, R., Liu, D., Qi, Y., Varticovski, L., and Sasisekharan, R. (2001). Probing Fibroblast Growth Factor Dimerization and Role of Heparin-Like Glycosaminoglycans in Modulating Dimerization and Signaling. J Biol Chem 276, 23421-23429.
251. Laird, A. D., Vajkoczy, P., Shawver, L. K., Thurnher, A., Liang, C., Mohammadi, M., Schlessinger, J., Ullrich, A., Hubbard, S. R., Blake, R. A., Fong, T. A., Strawn, L. M., Sun, L., Tang, C., Hawtin, R., Tang, F., Shenoy, N., Hirth, K. P., McMahon, G., and Cherrington (2000). SU6668 is a potent antiangiogenic and antitumor agent that induces regression of established tumors. Cancer Res 60, 4152-4160.
252. Landriscina, M., Bagala, C., Mandinova, A., Soldi, R., Micucci, I., Bellum, S., Prudovsky, I., and Maciag, T. (2001). Copper induces the assembly of a multiprotein aggregate implicated in the release of fibroblast growth factor 1 in response to stress. J Biol Chem 276, 25549-25557.
253. Langford, J. K., Stanley, M. J., Cao, D., and Sanderson, R. D. (1998). Multiple heparan sulfate chains are required for optimal syndecan-1 function. J Biol Chem 273, 29965-29971.
254. Lappi, D. A., Ying, W., Barthelemy, I., Martineau, D., Prieto, I., Benatti, L., Soria, M., and Baird, A. (1994). Expression and activities of a recombinant basic fibroblast growth factor-saporin fusion protein. J Biol Chem 269, 12552-12558.
255. Larrain, J., Alvarez, J., Hassell, J. R., and Brandan, E. (1997). Expression of perlecan, a proteoglycan that binds myogenic inhibitory basic fibroblast growth factor, is down regulated during skeletal muscle differentiation. Exp Cell Res 234, 405-412.
256. Laterre, P. F., Wittebole, X., and Dhainaut, J. F. (2003). Anticoagulant therapy in acute lung injury. Crit. Care Med 31, S329-336.
257. Laurent, T. C., and Fraser, J. R. (1992). Hyaluronan. Faseb J 6, 2397-2404.
258. Ledley, F. D. (1995). Nonviral gene therapy: the promise of genes as pharmaceutical products. Hum Gene Ther 6, 1129-1144.
259. Lehr, H. A., Bittinger, F., and Kirkpatrick, C. J. (2000). Microcirculatory dysfunction in sepsis: a pathogenetic basis for therapy? J Pathol 190, 373-386.
260. Leung, H. Y., Dickson, C., Robson, C. N., and Neal, D. E. (1996). Over-expression of fibroblast growth factor-8 in human prostate cancer. Oncogene 12, 1833-1835.
261. Ley, K. (2001). Pathways and bottlenecks in the web of inflammatory adhesion molecules and chemoattractants. Immunol Res 24, 87-95.
262. Li, Q., Park, P. W., Wilson, C. L., and Parks, W. C. (2002). Matrilysin shedding of syndecan-1 regulates chemokine mobilization and transepithelial efflux of neutrophils in acute lung injury. Cell 111, 635-646.
263. Liaw, P. C., Becker, D. L., Stafford, A. R., Fredenburgh, J. C., and Weitz, J. I. (2001). Molecular basis for the susceptibility of fibrin-bound thrombin to inactivation by heparin cofactor ii in the presence of dermatan sulfate but not heparin. J Biol Chem 276, 20959-20965.
264. Lim, Y., Kim, S. M., Lee, Y., Lee, W., Yang, T., Lee, M., Suh, H., and Park, J. (2001). Cationic hyperbranched poly(amino ester): a novel class of DNA condensing molecule with cationic surface, biodegradable three-dimensional structure, and tertiary amine groups in the interior. J Am Chem Soc 123, 2460-2461.
265. Lim, Y. B., Han, S. O., Kong, H. U., Lee, Y., Park, J. S., Jeong, B., and Kim, S. W. (2000). Biodegradable polyester, poly[alpha-(4-aminobutyl)-L-glycolic acid], as a non-toxic gene carrier. Pharm Res 17, 811-816.
266. Lim, Y. B., Kim, S. M., Suh, H., and Park, J. S. (2002). Biodegradable, endosome disruptive, and cationic network-type polymer as a highly efficient and nontoxic gene delivery carrier. Bioconjug Chem 13, 952-957.
267. Lin, L. L., Wartmann, M., Lin, A. Y., Knopf, J. L., Seth, A., and Davis R. J. (1993). cPLA2 is phosphorylated and activated by MAP kinase. Cell 72, 269-278.
268. Lin, X., Buff, E. M., Perrimon, N., and Michelson, A. M. (1999). Heparan sulfate proteoglycans are essential for FGF receptor signaling during Drosophila embryonic development. Development 126, 3715-3723.
269. Lindahl, U. (1990). Biosynthesis of heparin. Biochem Soc Trans 18, 803-805.
270. Linhardt, R. J., Galliher, P. M., and Cooney, C. L. (1986). Polysaccharide lyases. Appl Biochem Biotechnol 12, 135-176.
271. Linhardt, R. J. (2004). Heparin-induced cancer cell death. Chem Biol 11, 420-422.
272. Liotta, L. A., and Kohn, E. C. (2001). The microenvironment of the tumour-host interface. Nature 411, 375-379.
273. Little, S. R., Lynn, D. M., Ge, Q., Anderson, D. G., Puram, S. V., Chen, J., Eisen, H. N., and Langer, R. (2004). Poly-beta amino ester-containing microparticles enhance the activity of nonviral genetic vaccines. Proc Natl Acad Sci USA 101, 9534-9539.
274. Liu, D., Shriver, Z., Venkataraman, G., El Shabrawi, Y., and Sasisekharan, R. (2002). Tumor cell surface heparan sulfate as cryptic promoters or inhibitors of tumor growth and metastasis. Proc Natl Acad Sci USA 99, 568-573.
275. Liu, Y., Stack, S. M., Lakka, S. S., Khan, A. J., Woodley, D. T., Rao, J. S., and Rao, C. N. (1999). Matrix localization of tissue factor pathway inhibitor-2/matrix-associated serine protease inhibitor (TFPI-2/MSPI) involves arginine-mediated ionic interactions with heparin and dermatan sulfate: heparin accelerates the activity of TFPI-2/MSPI toward plasmin. Arch Biochem Biophys 370, 112-118.
276. Lofas, S., and Johnsson, B. (1990). A novel hydrogel matrix on gold surfaces in surface-plasmon resonance sensors for fast and efficient covalent immobilization of ligands. J Chem Soc Chem Commun, 1526-1528.
277. Lohmander, L. S., De Luca, S., Nilsson, B., Hascall, V. C., Caputo, C. B., Kimura, J. H., and Heinegard, D. (1980). Oligosaccharides on proteoglycans from the swarm rat chondrosarcoma. J Biol Chem 255, 6084-6091.
278. Lortat-Jacob, H., and Grimaud, J. A. (1991). Interferon-gamma C-terminal function: new working hypothesis. Heparan sulfate and heparin, new targets for IFN-gamma, protect, relax the cytokine and regulate its activity. Cell Mol Biol 37, 253-260.
279. Lundholm, K., Hulten, L., Engaras, B., Nordgren, S., and Svaninger, G. (1994). [Laparoscopy is not suitable in malignant tumors. Risk of neoplasm seeding by instrument]. Lakartidningen 91, 4262-4265.
280. Lundin, L., Larsson, H., Kreuger, J., Kanda, S., Lindahl, U., Salmivirta, M., and Claesson-Welsh, L. (2000). Selectively desulfated heparin inhibits fibroblast growth factor-induced mitogenicity and angiogenesis. J Biol Chem 275, 24653-24660.
281. Luo, D., Woodrow-Mumford, K., Belcheva, N., and Saltzman, W. M. (1999). Controlled DNA delivery systems. Pharm Res 16, 1300-1308.
282. Luo, D., and Saltzman, W. M. (2000). Synthetic DNA delivery systems. Nat Biotechnol 18, 33-37.
283. Luo, D., Han, E., Belcheva, N., and Saltzman, W. M. (2004). A self-assembled, modular DNA delivery system mediated by silica nanoparticles. J Control Release 95, 333-341.
284. Lush, C. W., and Kvietys, P. R. (2000). Microvascular dysfunction in sepsis. Microcirculation 7, 83-101.
285. Lynn, D. M., and Langer, R. (2000). Degradable Poly(β-amino esters): Synthesis, Characterization, and Self-assembly with plasmid DNA. J. Am. Chem. Soc 122, 10761-10768.
286. Lynn, D. M., Anderson, D. G., Putnam, D., and Langer, R. (2001). Accelerated Discovery of Synthetic Transfection Vectors: Parallel Synthesis and Screening of a Degradable Polymer Library. J. Am. Chem. Soc. 123, 8155-8156.
287. Lyon, M., Rushton, G., and Gallagher, J. T. (1997). The interaction of the transforming growth factor-betas with heparin/heparan sulfate is isoform-specific. J Biol Chem 272, 18000-18006.
288. Ma, J., Qiu, J., Hirt, L., Dalkara, T., and Moskowitz, M. A. (2001). Synergistic protective effect of caspase inhibitors and bFGF against brain injury induced by transient focal ischaemia. Br J Pharmacol 133, 345-350.
289. Maccarana, M., Casu, B., and Lindahl, U. (1993). Minimal sequence in heparin/heparan sulfate required for binding of basic fibroblast growth factor. J Biol Chem 268, 23898-23905.
290. Maheu, E., Ayral, X., and Dougados, M. (2002). A hyaluronan preparation (500-730 kDa) in the treatment of osteoarthritis: a review of clinical trials with Hyalgan. Int J Clin Pract 56, 804-813.
291. Maimone, M. M., and Tollefsen, D. M. (1991). Structure of a dermatan sulfate hexasaccharide that binds to heparin cofactor II with high affinity. J Biol Chem 266, 14830.
292. Majack, R. A., and Clowes, A. W. (1984). Inhibition of vascular smooth muscle cell migration by heparin-like glycosaminoglycans. Journal of Cell Physiol 188, 253-256.
293. Marshall, J. C. (2003). Such stuff as dreams are made on: mediator-directed therapy in sepsis. Nat Rev Drug Discov 2, 391-405.
294. Mason, M., Vercruysse, K. P., Kirker, K. R., Frisch, R., Marecak, D. M., Prestwich, G. D., and Pitt, W. G. (2000). Attachment of hyaluronic acid to polypropylene, polystyrene, and polytetrafluoroethylene. Biomaterials 21, 31-36.
295. Matter, K., and Balda, M. S. (2003). Signalling to and from tight junctions. Nat Rev Mol Cell Biol 4, 225-236.
296. McQuade, K. J., and Rapraeger, A. C. (2003). Syndecan-1 transmembrane and extracellular domains have unique and distinct roles in cell spreading. J Biol Chem.
297. Menger, M. D., and Vollmar, B. (2000). Role of microcirculation in transplantation. Microcirculation 7, 291-306.
298. Merry, C. L., Lyon, M., Deakin, J. A., Hopwood, J. J., and Gallagher, J. T. (1999). Highly sensitive sequencing of the sulfated domains of heparan sulfate. J Biol Chem 274, 18455-18462.
299. Mertens, G., Van der Schueren, B., van den Berghe, H., and David, G. (1996). Heparan sulfate expression in polarized epithelial cells: the apical sorting of glypican (GPI-anchored proteoglycan) is inversely related to its heparan sulfate content. J Cell Biol 132, 487-497.
300. Miceli, R., Hubert, M., Santiago, G., Yao, D. L., Coleman, T. A., Huddleston, K. A., and Connolly, K. (1999). Efficacy of keratinocyte growth factor-2 in dextran sulfate sodium-induced murine colitis. J Pharmacol Exp Ther 290, 464-471.
301. Michel, C. C., Phillips, M. E., and Turner, M. R. (1985). The effects of native and modified bovine serum albumin on the permeability of frog mesenteric capillaries. J Physiol 360, 333-346.
302. Michelacci, Y. M., and Dietrich, C. P. (1976). Chondroitinase C from Flavobacterium heparinum. J Biol Chem 251, 1154-1158.
303. Michelacci, Y. M., Horton, D. S., and Poblacion, C. A. (1987). Isolation and characterization of an induced chondroitinase ABC from Flavobacterium heparinum. Biochim Biophys Acta 923, 291-301.
304. Millane, R. P., Mitra, A. K., and Arnott, S. (1983). Chondroitin 4-sulfate: comparison of the structures of the potassium and sodium salts. J Mol Biol 169, 903-920.
305. Miller, R. A., and Britigan, B. E. (1995). The formation and biologic significance of phagocyte-derived oxidants. J Investig Med 43, 39-49.
306. Miranti, C. K., and Brugge, J. S. (2002). Sensing the environment: a historical perspective on integrin signal transduction. Nat Cell Biol 4, E83-90.
307. Mitra, A. K., Arnott, S., Atkins, E. D., and Isaac, D. H. (1983). Dermatan sulfate: molecular conformations and interactions in the condensed state. J Mol Biol 169, 873-901.
308. Miyake, K., Underhill, C. B., Lesley, J., and Kincade, P. W. (1990). Hyaluronate can function as a cell adhesion molecule and CD44 participates in hyaluronate recognition. J Exp Med 172, 69-75.
309. Mochizuki, Y., Tsuda, S., Kanetake, H., and Kanda, S. (2002). Negative regulation of urokinase-type plasminogen activator production through FGF-2-mediated activation of phosphoinositide 3-kinase. Oncogene 21, 7027-7033.
310. Montesano, R., Vassalli, J. D., Baird, A., Guillemin, R., and Orci, L. (1986). Basic fibroblast growth factor induces angiogenesis in vitro. Proc Natl Acad Sci USA 83, 7297-7301.
311. Morra, M., and Cassineli, C. (1999). Non-fouling properties of polysaccharide-coated surfaces. J Biomater Sci Polym Ed 10, 1107-1124.
312. Morra, M. (2000). On the molecular basis of fouling resistance. J Biomater Sci Polym Ed 11, 547-569.
313. Morra, M., Cassineli, C., Pavesop, A., and Renier, D. (2003). Atomic force microscopy evaluation of aquenous interfaces of immobilized hyaluron. J Colloid Interface Sci 259, 236-243.
314. Morris, T. A., Marsh, J. J., Konopka, R., Pedersen, C. A., and Chiles, P. G. (2000). Anti-thrombotic efficacies of enoxaparin, dalteparin, and unfractionated heparin in venous thrombo-embolism. Thromb Res 100, 185-194.
315. Morrison, J. A., Klingelhutz, A. J., and Raab-Traub, N. (2003). Epstein-Barr virus latent membrane protein 2A activates beta-catenin signaling in epithelial cells. J Virol 77, 12276-12284.
316. Morrison, J. A., Gulley, M. L., Pathmanathan, R., and Raab-Traub, N. (2004). Differential signaling pathways are activated in the Epstein-Barr virus-associated malignancies nasopharyngeal carcinoma and Hodgkin lymphoma. Cancer Res 64, 5251-5260.
317. Moy, F. J., Safran, M., Seddon, A. P., Kitchen, D., Bohlen, P., Aviezer, D., Yayon, A., and Powers, R. (1997). Properly oriented heparin-decasaccharide-induced dimers are the biologically active form of basic fibroblast growth factor. Biochemistry 36, 4782-4791.
318. Mulligan, R. C. (1993). The basic science of gene therapy. Science 260, 926-932.
319. Mulloy, B., and Forster, M. J. (2000). Conformation and dynamics of heparin and heparan sulfate. Glycobiology 10, 1147-1156.
320. Murphy-Ullrich, J. E., Westrick, L. G., Esko, J. D., and Mosher, D. F. (1988). Altered metabolism of thrombospondin by Chinese hamster ovary cells defective in glycosaminoglycan synthesis. J Biol Chem 263, 6400-6406.
321. Nader, H. B., Kobayashi, E. Y., Chavante, S. F., Tersariol, I. L., Castro, R. A., Shinjo, S. K., Naggi, A., Torri, G., Casu, B., and Dietrich, C. P. (1999). New insights on the specificity of heparin and heparan sulfate lyases from Flavobacterium heparinum revealed by the use of synthetic derivatives of K5 polysaccharide from E. coli and 2-O-desulfated heparin. Glycoconj J 16, 265-270.
322. Nakamoto, T., Chang, C. S., Li, A. K., and Chodak, G. W. (1992). Basic fibroblast growth factor in human prostate cancer cells. Cancer Res 52, 571-577.
323. Nanbo, A., Inoue, K., Adachi-Takasawa, K., and Takada, K. (2002). Epstein-Barr virus RNA confers resistance to interferon-alpha-induced apoptosis in Burkitt's lymphoma. Embo J 21, 954-965.
324. Naor, D., Sionov, R. V., and Ish-Shalom, D. (1997). CD44: structure, function, and association with the malignant process. Adv Cancer Res 71, 241-319.
325. Natarajan, V., Scribner, W. M., Morris, A. J., Roy, S., Vepa, S., Yang, J., Wadgaonkar, R., Reddy, S. P., Garcia, J. G., and Parinandi, N. L. (2001). Role of p38 MAP kinase in diperoxovanadate-induced phospholipase D activation in endothelial cells. Am J Physiol Lung Cell Mol Physiol 281, L435-449.
326. Natke, B., Venkataraman, G., Nugent, M. A., and Sasisekharan, R. (2000). Heparinase treatment of bovine smooth muscle cells inhibits fibroblast growth factor-2 binding to fibroblast growth factor receptor but not FGF-2 mediated cellular proliferation. Angiogenesis 3, 249-257.
327. Needham, L., Hellewell, P. G., Williams, T. J., and Gordon, J. L. (1988). Endothelial functional responses and increased vascular permeability induced by polycations. Lab Invest 59, 538-548.
328. Nelson, S. R., deSouza, N. M., and Allison, D. J. (2000). Endovascular stents and stent-grafts: is heparin coating desirable? Cardiovasc Intervent Radiol 23, 252-255.
329. Nelson, W. J., and Nusse, R. (2004). Convergence of Wnt, beta-catenin, and cadherin pathways. Science 303, 1483-1487.
330. Nieduszynski, I. A., Huckerby, T. N., Dickenson, J. M., Brown, G. M., Tai, G. H., and Bayliss, M. T. (1990). Structural aspects of skeletal keratan sulphates. Biochem Soc Trans 18, 792-793.
331. Nugent, M. A., and Edelman, E. R. (1992). Kinetics of basic fibroblast growth factor binding to its receptor and heparan sulfate proteoglycan: a mechanism for cooperactivity. Biochemistry 31, 8876-8883.
332. Nugent, M. A., Karnovsky, M. S., and Edelman, E. R. (1993). Vascular cell-derived heparan sulfate shows coupled inhibition of basic fibroblast growth factor binding and mitogenesis in vascular smooth muscle cells. Circ Res 73, 1051-1060.
333. Nugent, M. A., Nugent, H. M., Iozzo, R. V., Sanchack, K., and Edelman, E. R. (2000). Perlecan is required to inhibit thrombosis after deep vascular injury and contributes to endothelial cell-mediated inhibition of intimal hyperplasia. Proc Natl Acad Sci USA 97, 6722-6727.
334. Numa, F., Hirabayashi, K., Kawasaki, K., Sakaguchi, Y., Sugino, N., Suehiro, Y., Suminami, Y., Hirakawa, H., Umayahara, K., Nawata, S., Ogata, H., and Kato, H. (2002). Syndecan-1 expression in cancer of the uterine cervix: association with lymph node metastasis. Int J Oncol 20, 39-43.
335. Nurcombe, V., Ford, M. D., Wildschut, J. A., and Bartlett, P. F. (1993). Developmental regulation of neural response to FGF-1 and FGF-2 by heparan sulfate proteoglycan. Science 260, 103-106.
336. Nurcombe, V., Smart, C. E., Chipperfield, H., Cool, S. M., Boilly, B., and Hondermarck, H. (2000). Journal of Biological Chemistry 275, 30009-30018.
337. Nusrat, A., Turner, J. R., and Madara, J. L. (2000). Molecular physiology and pathophysiology of tight junctions. IV. Regulation of tight junctions by extracellular stimuli: nutrients, cytokines, and immune cells. Am J Physiol Gastrointest Liver Physiol 279, G851-857.
338. Oeben, M., Keller, R., Stuhlsatz, H. W., and Greiling, H. (1987). Constant and variable domains of different disaccharide structure in corneal keratan sulphate chains. Biochem J 248, 85-93.
339. Oelschlager, C., Romisch, J., Staubitz, A., Stauss, H., Leithauser, B., Tillmanns, H., and Holschermann, H. (2002). Antithrombin III inhibits nuclear factor kappaB activation in human monocytes and vascular endothelial cells. Blood 99, 4015-4020.
340. Oerther, S., Le Gall, H., Payan, E., Lapicque, F., Presle, N., Hubert, P., Dexheimer, J., and Netter, P. (1999). Hyaluronate-alginate gel as a novel biomaterial: mechanical properties and formation mechanism. Biotechnol Bioeng 63, 206-215.
341. Olofsson, A. M., Vestberg, M., Herwald, H., Rygaard, J., David, G., Arfors, K. E., Linde, V., Flodgaard, H., Dedio, J., Muller-Esterl, W., and Lundgren-Akerlund, E. (1999). Heparin-binding protein targeted to mitochondrial compartments protects endothelial cells from apoptosis. J Clin Invest 104, 885-894.
342. Omata, F., Birkenbach, M., Matsuzaki, S., Christ, A. D., and Blumberg, R. S. (2001). The expression of IL-12 p40 and its homologue, Epstein-Barr virus-induced gene 3, in inflammatory bowel disease. Inflamm Bowel Dis 7, 215-220.
343. Ong, S. H., Guy, G. R., Hadari, Y. R., Laks, S., Gotoh, N., Schlessinger, J., and Lax, I. (2000). FRS2 proteins recruit intracellular signaling pathways by binding to diverse targets on fibroblast growth factor and nerve growth factor receptors. Mol Cell Biol 20, 979-989.
344. Ong, S. H., Hadari, Y. R., Gotoh, N., Guy, G. R., Schlessinger, J., and Lax, I. (2001). Stimulation of phosphatidylinositol 3-kinase by fibroblast growth factor receptors is mediated by coordinated recruitment of multiple docking proteins. Proc Natl Acad Sci USA 98, 6074-6079.
345. O'Reilly, M. S., Boehm, T., Shing, Y., Fukai, N., Vasios, G., Lane, W. S., Flynn, E., Birkhead, J. R., Olsen, B. R., and Folkman, J. (1997). Endostatin: an endogenous inhibitor of angiogenesis and tumor growth. Cell 88, 277-285.
346. Ornitz, D. M., Yayon, A., Flanagan, J. G., Svahn, C. M., Levi, E., and Leder, P. (1992). Heparin is required for cell-free binding of basic fibroblast growth factor to a soluble receptor and for mitogenesis in whole cells. Mol Cell Biol 12, 240-247.
347. Ornitz, D. M., Herr, A. B., Nilsson, M., Westman, J., Svahn, C. M., and Waksman, G. (1995). FGF binding and FGF receptor activation by synthetic heparan-derived di- and trisaccharides. Science 268, 432-436.
348. Ornitz, D. M., Xu, J., Colvin, J. S., McEwen, D. G., MacArthur, C. A., Coulier, F., Gao, G., and Goldfarb, M. (1996). Receptor specificity of the fibroblast growth factor family. J Biol Chem 271, 15292-15297.
349. Ornitz, D. M., and Itoh, N. (2001). Fibroblast growth factors. Genome Biol 2, REVIEWS3005.
350. Ornitz, D. M., Herr, A. B., Nilsson, M., Westman, J., Scahn, C. M., an Waksman, G. (1995). FGF binding and FGF receptor activation by synthetic heparan-derived di- and trisaccharides. Science 268, 432-436.
351. Ornitz, D. M., Xu, J., Colvin, J. S., McEwen, D. G., MacArthur, C. A., Coulier, F., Gao, G., and Goldfarb, M. (1996). Receptor specificity of the fibroblast growth factor family. J Biol Chem 271, 15292-15297.
352. Osterberg, E., Bergstrom, K., Holmberg, K., Riggs, J. A., Vanalstine, J. M., Schuman, T. P., Burns, N. L., and Harris, J. M. (1993). Comparison of polysaccharide and poly(ethylene glycol) coatings for reduction of protein adsorption on polystyrene surfaces. Colloids Surf A 77, 159-169.
353. Osterberg, E., Bergstrom, K., Holmberg, K., Schuman, T. P., Riggs, J. A., Burns, N. L., Van Alstine, J. M., and Harris, J. M. (1995). Protein-rejecting ability of surface-bound dextran in end-on and side-on configurations: comparison to PEG. Biomed Mater Res 29, 741-747.
354. Ostrovsky, O., Berman, B., Gallagher, J., Mulloy, B., Fernig, D. G., Delehedde, M., and Ron, D. (2002). Differential effects of heparin saccharides on the formation of specific fibroblast growth factor (FGF) and FGF receptor complexes. J Biol Chem 277, 2444-2453.
355. Ostrovsky, O., Berman, B., Gallagher, J., Mulloy, B., Fernig, D. G., Delehedde, M., and Ron, D. (2001). Differential effects of heparin saccharides on the formation of specific fibroblast growth factor (FGF) and FGF receptor complexes. J Biol Chem 277, 2444-2453.
356. Ozen, M., Giri, D., Ropiquet, F., Mansukhani, A., and Ittmann, M. (2001). Role of fibroblast growth factor receptor signaling in prostate cancer cell survival. J Natl Cancer Inst 93, 1783-1790.
357. Paavonen, K., Puolakkainen, P., Jussila, L., Jahkola, T., and Alitalo, K. (2000). Vascular endothelial growth factor receptor-3 in lymphangiogenesis in wound healing. Am J Pathol 156, 1499-1504.
358. Padera, R., Venkataraman, G., Berry, D., Godvarti, R., and Sasisekharan, R. (1999). FGF-2/fibroblast growth factor receptor/heparin-like glycosaminoglycan interactions: a compensation model for FGF-2 signaling. Faseb J 13, 1677-1687.
359. Panyam, J., Zhou, W. Z., Prabha, S., Sahoo, S. K., and Labhasetwar, V. (2002). Rapid endo-lysosomal escape of poly(DL-lactide-co-glycolide) nanoparticles: implications for drug and gene delivery. Faseb J 16, 1217-1226.
360. Park, P. W., Pier, G. B., Hinkes, M. T., and Bernfield, M. (2001). Exploitation of syndecan-1 shedding by Pseudomonas aeruginosa enhances virulence. Nature 411, 98-102.
361. Park, Y., Yu, G., Gunay, N. S., and Linhardt, R. J. (1999). Purification and characterization of heparan sulfate proteoglycan from bovine brain. Biochem J 344, 723-730.
362. Patterson, C. E., Stasek, J. E., Schaphorst, K. L., Davis, H. W., and Garcia, J. G. (1995). Mechanisms of pertussis toxin-induced barrier dysfunction in bovine pulmonary artery endothelial cell monolayers. Am J Physiol 268, L926-934.
363. Pedersen, L. C., Tsuchida, K., Kitagawa, H., Sugahara, K., Darden, T. A., and Negishi, M. (2000). Heparan/chondroitin sulfate biosynthesis. Structure and mechanism of human glucuronyltransferase I. J Biol Chem 275, 34580-34585.
364. Pei, M., Solchaga, L. A., Seidel, J., Zeng, L., Vunjak-Novakovic, G., Caplan, A. I., and Freed, L. E. (2002). Bioreactors mediate the effectiveness of tissue engineering scaffolds. Faseb J 16, 1691-1694.
365. Pellegrini, L., Burke, D. F., von Delft, F., Mulloy, B., and Blundell, T. L. (2000). Crystal structure of fibroblast growth factor receptor ectodomain bound to ligand and heparin. Nature 407, 1029-1034.
366. Penc, S. F., Pomahac, B., Winkler, T., Dorschner, R. A., Eriksson, E., Herndon, M., and Gallo, R. L. (1998). Dermatan sulfate released after injury is a potent promoter of fibroblast growth factor-2 function. J Biol Chem 273, 28116-28121.
367. Pereira, H. A., Shafer, W. M., Pohl, J., Martin, L. E., and Spitznagel, J. K. (1990). CAP37, a human neutrophil-derived chemotactic factor with monocyte specific activity. J Clin Invest 85, 1468-1476.
368. Pereira, H. A., Erdem, I., Pohl, J., and Spitznagel, J. K. (1993). Synthetic bactericidal peptide based on CAP37: a 37-kDa human neutrophil granule-associated cationic antimicrobial protein chemotactic for monocytes. Proc Natl Acad Sci USA 90, 4733-4737.
369. Perez-Moreno, M., Jamora, C., and Fuchs, E. (2003). Sticky business: orchestrating cellular signals at adherens junctions. Cell 112, 535-548.
370. Perrimon, N., and Bernfield, M. (2000). Specificities of heparan sulphate proteoglycans in developmental processes. Nature 404, 725-728.
371. Petersen, O. W., Ronnov-Jessen, L., Howlett, A. R., and Bissell, M. J. (1992). Interaction with basement membrane serves to rapidly distinguish growth and differentiation pattern of normal and malignant human breast epithelial cells. Proc Natl Acad Sci USA 89, 9064-9068.
372. Petitou, M., Herault, J. P., Bernat, A., Driguez, P. A., Duchaussoy, P., Lormeau, J. C., and Herbert, J. M. (1999). Synthesis of thrombin-inhibiting heparin mimetics without side effects. Nature 398, 417-422.
373. Piacquadio, D., Jarcho, M., and Goltz, R. (1997). Evaluation of hylan b gel as a soft-tissue augmentation implant material. J Am Acad Dermatol 36, 544-549.
374. Picart, C., Lavalle, P., Hubert, P., Cuisineier, F. J. G., Decher, G., Schaff, P., and Voegel, J. C. (2001). Buildup mechanism for poly(L-lysine)/hyaluronic acid films onto a solid surface. Langmuir 17, 7414-7424.
375. Picone, O., Aucouturier, J. S., Louboutin, A., Coscas, Y., and Camus, E. (2003). Abdominal wall metastasis of a cervical adenocarcinoma at the laparoscopic trocar insertion site after ovarian transposition: case report and review of the literature. Gynecol Oncol 90, 446-449.
376. Piehler, J., Brecht, A., Hehl, K., and Gauglity, G. (1999). Protein interactions in covalently attached dextran layers. Colloids Surf B 13, 325-336.
377. Piepkorn, M. W., and Daynes, R. A. (1983). Heparin effect on DNA synthesis in a murine fibrosarcoma cell line: influence of anionic density. J Natl Cancer Inst 71, 615-618.
378. Plotnikov, A. N., Schlessinger, J., Hubbard, S. R., and Mohammadi, M. (1999). Structural basis of FGF receptor dimerization and activation. Cell 98, 641-650.
379. Plotnikov, A. N., Hubbard, S. R., Schlessinger, J., and Mohammdi, M. (2000). Crystal structures of two FGF-FGFR complexes reveal the determinants of ligand-receptor specificity. Cell 101, 413-424.
380. Podolsky, D. K. (2002). Inflammatory bowel disease. N Engl J Med 347, 417-429.
381. Polnaszek, N., Kwabi-Addo, B., Peterson, L. E., Ozen, M., Greenberg, N. M., Ortega, S., Basilico, C., and Ittmann, M. (2003). Fibroblast growth factor 2 promotes tumor progression in an autochthonous mouse model of prostate cancer. Cancer Res 63, 5754-5760.
382. Poste, G., and Fidler, I. J. (1980). The pathogenesis of cancer metastasis. Nature 283, 139-146.
383. Powell, A. K., Fernig, D. G., and Turnbull, J. E. (2002). Fibroblast growth factor receptors 1 and 2 interact differently with heparin/heparan sulfate. J Biol Chem 277, 28554-28563.
384. Powers, M. R., Blumenstock, F. A., Cooper, J. A., and Malik, A. B. (1989). Role of albumin arginyl sites in albumin-induced reduction of endothelial hydraulic conductivity. J Cell Physiol 141, 558-564.
385. Pries, A. R., Secomb, T. W., and Gaehtgens, P. (2000). The endothelial surface layer. Pflugers Arch 440, 653-666.
386. Pupa, S. M., Menard, S., Forti, S., and Tagliabue, E. (2002). New insights into the role of extracellular matrix during tumor onset and progression. J Cell Physiol 192, 259-267.
387. Putnam, D., Gentry, C. A., Pack, D. W., and Langer, R. (2001). Polymer-based gene delivery with low cytotoxicity by a unique balance of side-chain termini. Proc Natl Acad Sci USA 98, 1200-1205.
388. Pye, D. A., Vives, R. R., Turnbull, J. E., Hyde, P., and Gallagher, J. T. (1998). Heparan sulfate oligosaccharides require 6-O-sulfation for promotion of basic fibroblast growth factor mitogenic activity. J Biol Chem 273, 22936-22942.
389. Pye, D. A., Vives, R. R., Hyde, P., and Gallagher, J. T. (2000). Regulation of FGF-1 mitogenic activity by heparan sulfate oligosaccharides is dependent on specific structural features: differential requirements for the modulation of FGF-1 and FGF-2. Glycobiology 10, 1183-1192.
390. Qi, J. H., Matsumoto, T., Huang, K., Olausson, K., Christofferson, R., and Claesson-Welsh, L. (1999). Phosphoinositide 3 kinase is critical for survival, mitogenesis and migration but not for differentiation of endothelial cells. Angiogenesis 3, 371-380.
391. Qiang, Y. W., Endo, Y., Rubin, J. S., and Rudikoff, S. (2003). Wnt signaling in B-cell neoplasia. Oncogene 22, 1536-1545.
392. Raman, R., Venkataraman, G., Ernst, S., Sasisekharan, V., and Sasisekharan, R. (2003). Structural specificity of heparin binding in the fibroblast growth factor family of proteins. Proc Natl Acad Sci USA 100, 2357-2362.
393. Ramaswamy, S., Ross, K. N., Lander, E. S., and Golub, T. R. (2003). A molecular signature of metastasis in primary solid tumors. Nat Genet 33, 49-54.
394. Rao, D. S., Hyun, T. S., Kumar, P. D., Mizukami, I. F., Rubin, M. A., Lucas, P. C., Sanda, M. G., and Ross, T. S. (2002). Huntingtin-interacting protein 1 is overexpressed in prostate and colon cancer and is critical for cellular survival. J Clin Invest 110, 351-360.
395. Rapraeger, A. C., Kruffka, A., and Olwin, B. B. (1991). Requirement of heparin sulfate for bFGF-mediated fibroblast growth and myoblast differentiation. Science 252, 1705-1708.
396. Rapraeger, A. C. (1993). The coordinated regulation of heparan sulfate, syndecans and cell behavior. Curr Opin Cell Biol 5, 844-853.
397. Rapraeger, A. C. (1995). In the clutches of proteoglycans: how does heparan sulfate regulate FGF binding? Chem Biol 2, 645-649.
398. Ren, J. M., and Finklestein, S. P. (1997). Time window of infarct reduction by intravenous basic fibroblast growth factor in focal cerebral ischemia. Eur J Pharmacol 327, 11-16.
399. Rhomberg, A. J., Ernst, S., Sasisekharan, R., and Biemann, K. (1998). Mass spectrometric and capillary electrophoretic investigation of the enzymatic degradation of heparin-like glycosaminoglycans. Proc Natl Acad Sci USA 95, 4176-4181.
400. Robinson, C. J., and Stringer, S. E. (2001). The splice variants of vascular endothelial growth factor (VEGF) and their receptors. J Cell Sci 114, 853-865.
401. Robinson, K. A., Roubin, G. S., Siegel, R. J., Black, A. J., Apkarian, R. P., and King, S. B., 3rd (1988). Intra-arterial stenting in the atherosclerotic rabbit. Circulation 78, 646-653.
402. Roghani, M., Mansukhani, A., Dell'Era, P., Bellosta, P., Basilico, C., Rifkin, D. B., and Moscatelli, D. (1994). Heparin increases the affinity of basic fibroblast growth factor for its receptor but is not required for binding. J Biol Chem 269, 3976-3984.
403. Ropiquet, F., Giri, D., Lamb, D. J., and Ittmann, M. (1999). FGF7 and FGF2 are increased in benign prostatic hyperplasia and are associated with increased proliferation. J Urol 162, 595-599.
404. Ropiquet, F., Giri, D., Kwabi-Addo, B., Mansukhani, A., and Ittmann, M. (2000). Increased expression of fibroblast growth factor 6 in human prostatic intraepithelial neoplasia and prostate cancer. Cancer Res 60, 4245-4250.
405. Ropiquet, F., Giri, D., Kwabi-Addo, B., Schmidt, K., and Ittmann, M. (2000). FGF-10 is expressed at low levels in the human prostate. Prostate 44, 334-338.
406. Rops, A. L., van der Vlag, J., Lensen, J. F., Wijnhoven, T. J., van den Heuvel, L. P., van Kuppevelt, T. H., and Berden, J. H. (2004). Heparan sulfate proteoglycans in glomerular inflammation. Kidney Int 65, 768-785.
407. Rosini, P., Bonaccorsi, L., Baldi, E., Chiasserini, C., Forti, G., De Chiara, G., Lucibello, M., Mongiat, M., Iozzo, R. V., Garaci, E., Cozzolino, F., and Torcia, M. G. (2002). Androgen receptor expression induces FGF2, FGF-binding protein production, and FGF2 release in prostate carcinoma cells: role of FGF2 in growth, survival, and androgen receptor down-modulation. Prostate 53, 310-321.
408. Roskelley, C. D., Srebrow, A., and Bissell, M. J. (1995). A hierarchy of ECM-mediated signalling regulates tissue-specific gene expression. Curr Opin Cell Biol 7, 736-747.
409. Ross, R. (1993). The pathogenesis of atherosclerosis: a perspective for the 1990s. Nature 362, 801-809.
410. Ross, T. S., and Gilliland, D. G. (1999). Transforming properties of the Huntingtin interacting protein 1/platelet-derived growth factor beta receptor fusion protein. J Biol Chem 274, 22328-22336.
411. Rostand, K. S., and Esko, J. D. (1997). Microbial adherence to and invasion through proteoglycans. Infect Immun 65, 1-8.
412. Ruf, I. K., Rhyne, P. W., Yang, H., Borza, C. M., Hutt-Fletcher, L. M., Cleveland, J. L., and Sample, J. T. (1999). Epstein-barr virus regulates c-MYC, apoptosis, and tumorigenicity in Burkitt lymphoma. Mol Cell Biol 19, 1651-1660.
413. Ruf, I. K., Rhyne, P. W., Yang, H., Borza, C. M., Hutt-Fletcher, L. M., Cleveland, J. L., and Sample, J. T. (2001). EBV regulates c-MYC, apoptosis, and tumorigenicity in Burkitt's lymphoma. Curr Top Microbiol Immunol 258, 153-160.
414. Saksela, O., Moscatelli, D., Sommer, A., and Rifkin, D B. (1988). Endothelial cell-derived heparan sulfate binds basic fibroblast growth factor and protects it from proteolytic degradation. J Cell Biol 107, 743-751.
415. Sandborn, W. J., Sands, B. E., Wolf, D. C., Valentine, J. F., Safdi, M., Katz, S., Isaacs, K. L., Wruble, L. D., Katz, J., Present, D. H., Loftus, E. V., Jr., Graeme-Cook, F., Odenheimer, D. J., and Hanauer, S. B. (2003). Repifermin (keratinocyte growth factor-2) for the treatment of active ulcerative colitis: a randomized, double-blind, placebo-controlled, dose-escalation trial. Aliment Pharmacol Ther 17, 1355-1364.
416. Sanderson, R. D., Turnbull, J. E., Gallagher, J. T., and Lander, A. D. (1994). Fine structure of heparan sulfate regulates syndecan-1 function and cell behavior. J Biol Chem 269, 13100-13106.
417. Sanderson, R. D. (2001). Heparan sulfate proteoglycans in invasion and metastasis. Semin Cell Dev Biol 12, 89-98.
418. Sandset, P. M., Bendz, B., and Hansen, J. B. (2000). Physiological function of tissue factor pathway inhibitor and interaction with heparins. Haemostasis 30 Suppl 2, 48-56.
419. Sannes, P. L., Khosla, J., Li, C. M., and Pagan, I. (1998). Sulfation of extracellular matrices modifies growth factor effects on type II cells on laminin substrata. Am J Physiol 275, L701-708.
420. Saoncella, S., Echtermeyer, F., Denhez, F., Nowlen, J. K., Mosher, D. F., Robinson, S. D., Hynes, R. O., and Goetinck, P. F. (1999). Syndecan-4 signals cooperatively with integrins in a Rho-dependent manner in the assembly of focal adhesions and actin stress fibers. Proc Natl Acad Sci USA 96, 2805-2810.
421. Sasaki, T., Larsson, H., Kreuger, J., Salmivirta, M., Claesson-Welsh, L., Lindahl, U., Hohenester, E., and Timpl, R. (1999). Structural basis and potential role of heparin/heparan sulfate binding to the angiogenesis inhibitor endostatin. Embo J 18, 6240-6248.
422. Sasisekharan, R., Bulmer, M., Moremen, K. W., Cooney, C. L., and Langer, R. (1993). Cloning and expression of heparinase I gene from Flavobacterium heparinum. Proc Natl Acad Sci USA 90, 3660-3664.
423. Sasisekharan, R., Moses, M. A., Nugent, M. A., Cooney, C. L., and Langer, R. (1994). Heparinase inhibits neovascularization. Proc Natl Acad Sci USA 91, 1524-1528.
424. Sasisekharan, R., Leckband, D., Godavarti, R., Venkataraman, G., Cooney, C. L., and Langer, R. (1995). Heparinase I from Flavobacterium heparinum: the role of the cysteine residue in catalysis as probed by chemical modification and site-directed mutagenesis. Biochemistry 34, 14441-14448.
425. Sasisekharan, R., Venkataraman, G., Godavarti, R., Ernst, S., Cooney, C. L., and Langer, R. (1996). Heparinase I from Flavobacterium heparinum. Mapping and characterization of the heparin binding domain. J Biol Chem 271, 3124-3131.
426. Sasisekharan, R., and Venkataraman, G. (2000). Heparin and heparan sulfate: biosynthesis, structure and function. Curr Opin Chem Biol 4, 626-631.
427. Sasisekharan, R., Shriver, Z., Venkataraman, G., and Narayanasami, U. (2002). Roles of heparan-sulphate glycosaminoglycans in cancer. Nat Rev Cancer 2, 521-528.
428. Sato, N., Shimada, M., Nakajima, H., Oda, H., and Kimura, S. (1994). Cloning and expression in Escherichia coli of the gene encoding the Proteus vulgaris chondroitin ABC lyase. Appl Microbiol Biotechnol 41, 39-46.
429. Saumon, G., Soler, P., and Martet, G. (1995). Effect of polycations on barrier and transport properties of alveolar epithelium in situ. Am J Physiol 269, L185-194.
430. Schittny, J. C., and Yurchenco, P. D. (1989). Basement membranes: molecular organization and function in development and disease. Curr Opin Cell Biol 1, 983-988.
431. Schlessinger, J., Plotnikov, A. N., Ibrahimi, O. A., Eliseenkova, A. V., Yeh, B. K., Yayon, A., Linhardt, R. J., and Mohammadi, M. (2000). Crystal structure of a ternary FGF-FGFR-heparin complex reveals a dual role for heparin in FGFR binding and dimerization. Mol Cell 6, 743-750.
432. Schmidtchen, A., Frick, I. M., and Bjorck, L. (2001). Dermatan sulphate is released by proteinases of common pathogenic bacteria and inactivates antibacterial alpha-defensin. Mol Microbiol 39, 708-713.
433. Schneider, G. B., Kurago, Z., Zaharias, R., Gruman, L. M., Schaller, M. D., and Hendrix, M. J. (2002). Elevated focal adhesion kinase expression facilitates oral tumor cell invasion. Cancer 95, 2508-2515.
434. Scholle, F., Bendt, K. M., and Raab-Traub, N. (2000). Epstein-Barr virus LMP2A transforms epithelial cells, inhibits cell differentiation, and activates Akt. J Virol 74, 10681-10689.
435. Schonherr, E., and Hausser, H. J. (2000). Extracellular matrix and cytokines: a functional unit. Dev Immunol 7, 89-101.
436. Sebestyen, A., Totth, A., Mihalik, R., Szakacs, O., Paku, S., and Kopper, L. (2000). Syndecan-1-dependent homotypic cell adhesion in HT58 lymphoma cells. Tumour Biol 21, 349-357.
437. Sengupta, K., Schilling, J., Marx, S., Markus, F., and Sackmann, E. (2003). Supported membrane coupled ultra-thin layer of hyaluronic acid: viscoelastic properties of a tissue-surface mimetic system. Biophysical J 84, 381a.
438. Shard, A. G., Davies, M. C., Tendler, J. J. B., Benedetti, L., Purbrick, M. D., Paul, A., and Beamson, G. (1997). X-ray photoelectron spectroscopy and time-of-flight SIMS investigations of hyaluronic acid derivatives. Langmuir 13, 2080-2014.
439. Shibasaki, Y., Seki, A., and Teishi, N. (1995). Thermoanalytical study on anchoring effects of long-chain diynoic acids in thermal polymerization. Thermochimica Acta 253, 103-110.
440. Shriver, Z., Hu, Y., Pojasek, K., and Sasisekharan, R. (1998). Heparinase II from Flavobacterium heparinum. Role of cysteine in enzymatic activity as probed by chemical modification and site-directed mutagenesis. J Biol Chem 273, 22904-22912.
441. Shriver, Z., Hu, Y., and Sasisekharan, R. (1998). Heparinase II from Flavobacterium heparinum. Role of histidine residues in enzymatic activity as probed by chemical modification and site-directed mutagenesis. J Biol Chem 273, 10160-10167.
442. Shriver, Z., Liu, D., Hu, Y., and Sasisekharan, R. (1999). Biochemical investigations and mapping of the calcium-binding sites of heparinase I from Flavobacterium heparinum. J Biol Chem 274, 4082-4088.
443. Shriver, Z., Sundaram, M., Venkataraman, G., Fareed, J., Linhardt, R., Biemann, K., and Sasisekharan, R. (2000). Cleavage of the antithrombin III binding site in heparin by heparinases and its implication in the generation of low molecular weight heparin. Proc Natl Acad Sci USA 97, 10365-10370.
444. Shriver, Z., Liu, D., and Sasisekharan, R. (2002). Emerging views of heparan sulfate glycosaminoglycan structure/activity relationships modulating dynamic biological functions. Trends Cardiovasc Med 12, 71-77.
445. Sleeman, M., Fraser, J., McDonald, M., Yuan, S., White, D., Grandison, P., Kumble, K., Watson, J. D., and Murison, J. G. (2001). Identification of a new fibroblast growth factor receptor, FGFR5. Gene 271, 171-182.
446. Smetsers, T. F., van de Westerlo, E. M., ten Dam, G. B., Clarijs, R., Versteeg, E. M., van Geloof, W. L., Veerkamp, J. H., van Muijen, G. N., and van Kuppevelt, T. H. (2003). Localization and characterization of melanoma-associated glycosaminoglycans: differential expression of chondroitin and heparan sulfate epitopes in melanoma. Cancer Res 63, 2965-2970.
447. Sood, A. K., Coffin, J. E., Schneider, G. B., Fletcher, M. S., DeYoung, B. R., Gruman, L. M., Gershenson, D. M., Schaller, M. D., and Hendrix, M. J. (2004). Biological significance of focal adhesion kinase in ovarian cancer: role in migration and invasion. Am J Pathol 165, 1087-1095.
448. Sperinde, G. V., and Nugent, M. A. (2000). Mechanisms of fibroblast growth factor 2 intracellular processing: a kinetic analysis of the role of heparan sulfate proteoglycans. Biochemistry 39, 3788-3796.
449. Spivak-Kroizman, T., Lemmon, M. A., Dikic, I., Ladbury, J. E., Pinchasi, D., Huang, J., Jaye, M., Crumley, G., Schlessinger, J., and Lax, I. (1994). Heparin-induced oligomerization of FGF molecules is responsible for FGF receptor dimerization, activation, and cell proliferation. Cell 79, 1015-1024.
450. Stacker, S. A., Caesar, C., Baldwin, M. E., Thornton, G. E., Williams, R. A., Prevo, R., Jackson, D. G., Nishikawa, S., Kubo, H., and Achen, M. G. (2001). VEGF-D promotes the metastatic spread of tumor cells via the lymphatics. Nat Med 7, 186-191.
451. Stauber, D. J., DiGabriele, A. D., and Hendrickson, W. A. (2000). Structural interactions of fibroblast growth factor receptor with its ligands. Proc Natl Acad Sci USA 97, 49-54.
452. Stile, R. A., Barber, T. A., Castner, D. G., and Healy, K. E. (2002). Sequential robust design methodology and X-ray photoelectron spectroscopy to analyze the grafting of hyaluronic acid to glass substrates. J Biomed Mater Res 61, 391-398.
453. Strowski, M. Z., Cramer, T., Schafer, G., Juttner, S., Walduck, A., Schipani, E., Kemmner, W., Wessler, S., Wunder, C., Weber, M., Meyer, T. F., Wiedenmann, B., Jons, T., Naumann, M., and Hocker, M. (2003). Helicobacter pylori stimulates host vascular endothelial growth factor-A (vegf-A) gene expression via MEK/ERK-dependent activation of Sp1 and Sp3. Faseb J.
454. Sugahara, K., and Kitagawa, H. (2002). Heparin and heparan sulfate biosynthesis. IUBMB Life 54, 163-175.
455. Suh, K. Y., Yang, J. M., Khademhosseini, A., Berry, D., Tran, T. N., Park, H., and Langer, R. (2005). Characterization of chemisorbed hyaluronic acid directly immobilized on solid substrates. J Biomed Mater Res B Appl Biomater 72, 292-298.
456. Summerford, C., Bartlett, P. F., and Samulski, R. J. (1999). Alpha Vbeta5 integrin: a co-receptor for adeno-associated virus type 2 infection. Nat Med 5, 78-82.
457. Sundaram, M., Qi, Y., Shriver, Z., Liu, D., Zhao, G., Venkataraman, G., Langer, R., and Sasisekharan, R. (2003). Rational design of low-molecular weight heparins with improved in vivo activity. Proc Natl Acad Sci USA 100, 651-656.
458. Swanson, R. A., Morton, M. T., Tsao-Wu, G., Savalos, R. A., Davidson, C., and Sharp, F. R. (1990). A semiautomated method for measuring brain infarct volume. J Cereb Blood Flow Metab 10, 290-293.
459. Szabo, S., Folkman, J., Vattay, P., Morales, R. E., Pinkus, G. S., and Kato, K. (1994). Accelerated healing of duodenal ulcers by oral administration of a mutein of basic fibroblast growth factor in rats. Gastroenterology 106, 1106-1111.
460. Tanaka, Y., Adams, D. H., Hubscher, S., Hirano, H., Siebenlist, U., and Shaw, S. (1993). T-cell adhesion induced by proteoglycan-immobilized cytokine MIP-1 beta. Nature 361, 79-82.
461. Tapon-Bretaudiere, J., Drouet, B., Matou, S., Mourao, P. A., Bros, A., Letourneur, D., and Fischer, A. M. (2000). Modulation of vascular human endothelial and rat smooth muscle cell growth by a fucosylated chondroitin sulfate from echinoderm. Thromb Haemost 84, 332-337.
462. Teien, A. N., and Lie, M. (1975). Heparin assay in plasma: a comparison of five clotting methods. Thromb Res 7, 777-788.
463. Teien, A. N., Lie, M., and Abildgaard, U. (1976). Assay of heparin in plasma using a chromogenic substrate for activated factor X. Thromb Res 8, 413-416.
464. Teien, A. N., and Lie, M. (1977). Evaluation of an amidolytic heparin assay method: increased sensitivity by adding purified antithrombin III. Thromb Res 10, 399-410.
465. Teoh, K. H., Young, E., Bradley, C. A., and Hirsh, J. (1993). Heparin binding proteins. Contribution to heparin rebound after cardiopulmonary bypass. Circulation 88, 11420-425.
466. Thierry, B., Winnik, F. M., Merhi, Y., and Tabrizian, M. (2003). Nanocoatings onto arteries via layer-by-layer deposition: toward the in vivo repair of damaged blood vessels. J Am Chem Soc 125, 7494-7495.
467. Thodeti, C. K., Albrechtsen, R., Grauslund, M., Asmar, M., Larsson, C., Takada, Y., Mercurio, A. M., Couchman, J. R., and Wewer, U. M. (2003). ADAM12/syndecan-4 signaling promotes beta 1 integrin-dependent cell spreading through protein kinase Calpha and RhoA. J Biol Chem 278, 9576-9584.
468. Timpl, R. (1996). Macromolecular organization of basement membranes. Curr Opin Cell Biol 8, 618-624.
469. Tkachenko, E., and Simons, M. (2002). Clustering induces redistribution of syndecan-4 core protein into raft membrane domains. J Biol Chem 277, 19946-19951.
470. Tkachenko, E., Lutgens, E., Stan, R. V., and Simons, M. (2004). Fibroblast growth factor 2 endocytosis in endothelial cells proceed via syndecan-4-dependent activation of Rac1 and a Cdc42-dependent macropinocytic pathway. J Cell Sci 117, 3189-3199.
471. Toki, N., Tsukamoto, N., Kaku, T., Toh, N., Saito, T., Kamura, T., Matsukuma, K., and Nakano, H. (1991). Microscopic ovarian metastasis of the uterine cervical cancer. Gynecol Oncol 41, 46-51.
472. Torcia, M., Lucibello, M., De Chiara, G., Labardi, D., Nencioni, L., Bonini, P., Garaci, E., and Cozzolino, F. (1999). Interferon-alpha-induced inhibition of B16 melanoma cell proliferation: interference with the bFGF autocrine growth circuit. Biochem Biophys Res Commun 262, 838-844.
473. Travis, A. J., Merdiushev, T., Vargas, L. A., Jones, B. H., Purdon, M. A., Nipper, R. W., Galatioto, J., Moss, S. B., Hunnicutt, G. R., and Kopf, G. S. (2001). Expression and localization of caveolin-1, and the presence of membrane rafts, in mouse and Guinea pig spermatozoa. Dev Biol 240, 599-610.
474. Trowbridge, J. M., and Gallo, R. L. (2002). Dermatan sulfate: new functions from an old glycosaminoglycan. Glycobiology 12, 117R-125R.
475. Trowbridge, J. M., Rudisill, J. A., Ron, D., and Gallo, R. L. (2002). Dermatan sulfate binds and potentiates activity of keratinocyte growth factor (FGF-7). J Biol Chem 277, 42815-42820.
476. Tumova, S., Woods, A., and Couchman, J. R. (2000). Heparan sulfate proteoglycans on the cell surface: versatile coordinators of cellular functions. Int J Biochem and Cell Biol 32, 269-288.
477. Turnbull, J. E., Fernig, D. G., Ke, Y., Wilkinson, M. C., and Gallagher, J. T. (1992). Identification of the basic fibroblast growth factor binding sequence in fibroblast heparan sulfate. J Biol Chem 267, 10337-10341.
478. Turnbull, J. E., Hopwood, J. J., and Gallagher, J. T. (1999). A strategy for rapid sequencing of heparan sulfate and heparin saccharides. Proc Natl Acad Sci USA 96, 2698-2703.
479. Tyagi, M., Rusnati, M., Presta, M., and Giacca, M. (2001). Internalization of HIV-1 that requires cell surface heparan sulfate proteoglycans. J Biol Chem 276, 3254-3261.
480. Unger, E. F., Goncalves, L., Epstein, S. E., Chew, E. Y., Trapnell, C. B., Cannon, R. O. r., and Quyyumi, A. A. (2000). Effects of a single intracoronary injection of basic fibroblast growth factor in stable angina pectoris. Am J Cardiol 85, 1414-1419.
481. van de Westerlo, E. M., Smetsers, T. F., Dennissen, M. A., Linhardt, R. J., Veerkamp, J. H., van Muijen, G. N., and van Kuppevelt, T. H. (2002). Human single chain antibodies against heparin: selection, characterization, and effect on coagulation. Blood 99, 2427-2433.
482. van Kuppevelt, T. H., Dennissen, M. A., van Venrooij, W. J., Hoet, R. M., and Veerkamp, J. H. (1998). Generation and application of type-specific anti-heparan sulfate antibodies using phage display technology. Further evidence for heparan sulfate heterogeneity in the kidney. J Biol Chem 273, 12960-12966.
483. Varki, A. (1993). Biological roles of oligosaccharides: all of the theories are correct. Glycobiology 3, 97-130.
484. Varner, J. A., and Cheresh, D. A. (1996). Integrins and cancer. Curr Opin Cell Biol 8, 724-730.
485. Veikkola, T., Karkkainen, M., Claesson-Welsh, L., and Alitalo, K. (2000). Regulation of angiogenesis via vascular endothelial growth factor receptors. Cancer Res 60, 203-212.
486. Venkataraman, G., Sasisekharan, V., Herr, A. B., Ornitz, D. M., Waksman, G., Cooney, C. L., Langer, R., and Sasisekharan, R. (1996). Preferential self-association of basic fibroblast growth factor is stabilized by heparin during receptor dimerization and activation. Proc Natl Acad Sci USA 93, 845-850.
487. Venkataraman, G., Shriver, Z., Davis, J. C., and Sasisekharan, R. (1999). Fibroblast growth factors 1 and 2 are distinct in oligomerization in the presence of heparin-like glycosaminoglycans. Proc Natl Acad Sci USA 96, 1892-1897.
488. Venkataraman, G., Shriver, Z., Raman, R., and Sasisekharan, R. (1999). Sequencing Complex Polysaccharides. Science 286, 537-542.
489. Vepa, S., Scribner, W. M., and Natarajan, V. (1997). Activation of endothelial cell phospholipase D by polycations. Am J Physiol 272, L608-613.
490. Verin, A. D., Birukova, A., Wang, P., Liu, F., Becker, P., Birukov, K., and Garcia, J. G. (2001). Microtubule disassembly increases endothelial cell barrier dysfunction: role of MLC phosphorylation. Am J Physiol Lung Cell Mol Physiol 281, L565-574.
491. Vernon, R. B., and Sage, E. H. (1995). Between molecules and morphology. Extracellular matrix and creation of vascular form. Am J Pathol 147, 873-883.
492. Vlodavsky, I., Korner, G., Ishai-Michaeli, R., Bashkin, P., Bar-Shavit, R., and Fuks, Z. (1990). Extracellular matrix-resident growth factors and enzymes: possible involvement in tumor metastasis and angiogenesis. Cancer Metastasis Rev 9, 203-226.
493. Vlodavsky, I., Miao, H. Q., Medalion, B., Danagher, P., and Ron, D. (1996). Involvement of heparan sulfate and related molecules in sequestration and growth promoting activity of fibroblast growth factor. Cancer Metastasis Rev 15, 177-186.
494. Vlodavsky, I., Friedmann, Y., Elkin, M., Aingorn, H., Atzmon, R., Ishai-Michaeli, R., Bitan, M., Pappo, O., Peretz, T., Michal, I., Spector, L., and Pecker, I. (1999). Mammalian heparanase: gene cloning, expression and function in tumor progression and metastasis. Nat Med 5, 793-802.
495. Vlodavsky, I., and Friedmann, Y. (2001). Molecular properties and involvement of heparanase in cancer metastasis and angiogenesis. J Clin Invest 108, 341-347.
496. Vlodavsky, I., Goldshmidt, O., Zcharia, E., Atzmon, R., Rangini-Guatta, Z., Elkin, M., Peretz, T., and Friedmann, Y. (2002). Mammalian heparanase: involvement in cancer metastasis, angiogenesis and normal development. Semin Cancer Biol 12, 121-129.
497. Wada, K., Sugimori, H., Bhide, P. G., Moskowitz, M. A., and Finklestein, S. P. (2003). Effect of basic fibroblast growth factor treatment on brain progenitor cells after permanent focal ischemia in rats. Stroke 34, 2722-2728.
498. Wakisaka, N., Murono, S., Yoshizaki, T., Furukawa, M., and Pagano, J. S. (2002). Epstein-barr virus latent membrane protein 1 induces and causes release of fibroblast growth factor-2. Cancer Res 62, 6337-6344.
499. Walenga, J. M., Jeske, W. P., Bara, L., Sarnama, M. M., and Fareed, J. (1997). Biochemical and pharmacologic rationale for the development of a synthetic heparin pentasaccharide. Thromb Res 86, 1-36.
500. Wang, D., Liu, S., Trummer, B. J., Deng, C., and Wang, A. (2002). Carbohydrate microarrays for the recognition of cross-reactive molecular markers of microbes and host cells. Nat Biotechnol 20, 275-281.
501. Wang, H., Toida, T., Kim, Y. S., Capila, I., Hilemna, R. E., Bernfield, M., and Linhardt, R. J. (1997). Glycosaminoglycans can induce fibroblast growth factor-2 mitogenicity without significant growth factor binding. Biochem Biophys Res Commun 235, 369-373.
502. Wang, L., Malsch, R., and Harenberg, J. (1997). Heparins, low-molecular-weight heparins, and other glycosaminoglycans analyzed by agarose gel electrophoresis and azure A-silver staining. Semin Thromb Hemost 23, 11-16.
503. Wen, W., Moses, M. A., Wiederschain, D., Arbiser, J. L., and Folkman, J. (1999). The generation of endostatin is mediated by elastase. Cancer Res 59, 6052-6056.
504. Wong, P., and Burgess, W. H. (1998). FGF2-Heparin co-crystal complex-assisted design of mutants FGF1 and FGF7 with predictable heparin affinities. J Biol Chem 273, 18617-18622.
505. Woods, A., Couchman, J. R., Johansson, S., and Hook, M. (1986). Adhesion and cytoskeletal organisation of fibroblasts in response to fibronectin fragments. Embo J 5, 665-670.
506. Wu, W., Shu, X., Hovsepyan, H., Mosteller, R. D., and Broek, D. (2003). VEGF receptor expression and signaling in human bladder tumors. Oncogene 22, 3361-3370.
507. Xin, L., Xu, R., Zhang, Q., Li, T. P., and Gan, R. B. (2000). Kringle 1 of human hepatocyte growth factor inhibits bovine aortic endothelial cell proliferation stimulated by basic fibroblast growth factor and causes cell apoptosis. Biochem Biophys Res Commun 277, 186-190.
508. Yamada, S., Yoshida, K., Sugiura, M., Sugahara, K., Khoo, K. H., Morris, H. R., and Dell, A. (1993). Structural studies on the bacterial lyase-resistant tetrasaccharides derived from the antithrombin III-binding site of porcine intestinal heparin. J Biol Chem 268, 4780-4787.
509. Yamagata, M., Kimata, K., Oike, Y., Tani, K., Maeda, N., Yoshida, K., Shimomura, Y., Yoneda, M., and Suzuki, S. (1987). A monoclonal antibody that specifically recognizes a glucuronic acid 2-sulfate-containing determinant in intact chondroitin sulfate chain. J Biol Chem 262, 4146-4152.
510. Yan, G., Fukabori, Y., McBride, G., Nikolaropolous, S., and McKeehan, W. L. (1993). Exon switching and activation of stromal and embryonic fibroblast growth factor (FGF)-FGF receptor genes in prostate epithelial cells accompany stromal independence and malignancy. Mol Cell Biol 13, 4513-4522.
511. Yayon, A., Klagsbrun, M., Esko, J. D., Leder, P., and Ornitz, D. M. (1991). Cell surface, heparin-like molecules are required for binding of basic fibroblast growth factor to its high affinity receptor. Cell 64, 841-848.
512. Ye, S., Luo, Y., Lu, W., Jones, R. B., Linhardt, R. J., Capila, I., Toida, T., Kan, M., Pelletier, H., and McKeehan, W. L. (2001). Structural basis of interaction of FGF-1, FGF-2, and FGF-7 with different heparan sulfate motifs. Biochemistry 40, 14429-14439.
513. Yeaman, C., Grindstaff, K. K., and Nelson, W. J. (1999). New perspectives on mechanisms involved in generating epithelial cell polarity. Physiol Rev 79, 73-98.
514. Yeh, B. K., Igarashi, M., Eliseenkova, A. V., Plotnikov, A. N., Sher, I., Ron, D., Aaronson, S. A., and Mohammadi, M. (2003). Structural basis by which alternative splicing confers specificity in fibroblast growth factor receptors. Proc Natl Acad Sci USA 100, 2266-2271.
515. Yoshioka, T., Tsuru, K., Hayakawa, S., and Osaka, A. (2003). Preparation of alginic acid layers on stainless-steel substrates for biomedical applications. Biomaterials 24, 2889-2894.
516. Youakim, A., and Ahdieh, M. (1999). Interferon-gamma decreases barrier function in T84 cells by reducing ZO-1 levels and disrupting apical actin. Am J Physiol 276, G1279-G1288.
517. Yuge, T., Furukawa, A., Nakamura, K., Nagashima, Y., Shinozaki, K., Nakamura, T., and Kimura, R. (1997). Metabolism of the intravenously administered recombinant human basic fibroblast growth factor, trafermin, in liver and kidney: degradation implicated in its selective localization to the fenestrated type microvasculatures. Biol Pharm Bull 20, 786-793.
518. Yun, M. S., Kim, S. E., Jeon, S. H., Lee, J. S., and Choi, K. Y. (2004). Both ERK and Wnt/{beta}-catenin pathways are involved in Wnt3a-induced proliferation. J Cell Sci.
519. Yung, S., Woods, A., Chan, T. M., Davies, M., Williams, J. D., and Couchman, J. R. (2001). Syndecan-4 up-regulation in proliferative renal disease is related to microfilament organization. Faseb J 15, 1631-1633.
520. Yurchenco, P. D., and Schittny, J. C. (1990). Molecular architecture of basement membranes. Faseb J 4, 1577-1590.
521. Zacharski, L. R., Henderson, W. G., Rickles, F. R., Forman, W. B., Cornell, C. J., Jr., Forcier, R. J., Harrower, H. W., and Johnson, R. O. (1979). Rationale and experimental design for the VA Cooperative Study of Anticoagulation (Warfarin) in the Treatment of Cancer. Cancer 44, 732-741.
522. Zacharski, L. R., Henderson, W. G., Rickles, F. R., Forman, W. B., Cornell, C. J., Jr., Forcier, R. J., Edwards, R., Headley, E., Kim, S. H., O'Donnell, J. R., O'Dell, R., Tornyos, K., and Kwaan, H. C. (1981). Effect of warfarin on survival in small cell carcinoma of the lung. Veterans Administration Study No. 75. Jama 245, 831-835.
523. Zacharski, L. R., and Ornstein, D. L. (1998). Heparin and cancer. Thromb Haemost 80, 10-23.
524. Zauner, W., Brunner, S., Buschle, M., Ogris, M., and Wagner, E. (1999). Differential behaviour of lipid based and polycation based gene transfer systems in transfecting primary human fibroblasts: a potential role of polylysine in nuclear transport. Biochim Biophys Acta 1428, 57-67.
525. Zeeh, J. M., Procaccino, F., Hoffmann, P., Aukerman, S. L., McRoberts, J. A., Soltani, S., Pierce, G. F., Lakshmanan, J., Lacey, D., and Eysselein, V. E. (1996). Keratinocyte growth factor ameliorates mucosal injury in an experimental model of colitis in rats. Gastroenterology 110, 1077-1083.
526. Zhang, Z., Coomans, C., and G., D. (2001). Membrane heparan sulfate proteoglycan-supported FGF2-FGFR1 signaling: evidence in support of the “cooperative end structures” model. J Biol Chem 276, 41921-41929.
527. Zhou, F. Y., Owens, R. T., Hermonen, J., Jalkanen, M., and Hook, M. (1997). Is the sensitivity of cells for FGF-1 and FGF-2 regulated by cell surface heparan sulfate proteoglycans? Eur J Cell Biol 73, 166-174.
528. Zimmermann, P., and David, G. (1999). The syndecans, turners of transmembrane signaling. FASEB J 13, S91-S100.
529. Zimmermann, P., and David, G. (1999). The syndecans, tuners of transmembrane signaling. Faseb J 13 Suppl, S91-S100.
530. Zimmermann, P., Tomatis, D., Rosas, M., Grootjans, J., Leenaerts, I., Degeest, G., Reekmans, G., Coomans, C., and David, G. (2001). Characterization of syntenin, a syndecan-binding PDZ protein, as a component of cell adhesion sites and microfilaments. Mol Biol Cell 12, 339-350.
Achen M G, Jeltsch M, Kulk E, Makinen T, Vitali A, Wilks A F, Alitalo K, Stacker S A. 1998. Vascular endothelial growth factor D (VEGF-D) is a ligand for the tyrosine kinases VEGF receptor 2 (Flk1) and VEGF receptor 3 (Flt4). Proc Natl Acad Sci USA 95(2):548-553.
Allen B L, Filla M S, Rapraeger A C. 2001. Role of heparan sulfate as a tissue-specific regulator of FGF-4 and FGF receptor recognition. J Cell Biol 155(5):845-858.
Berry D, Kwan C P, Shriver Z, Venkataraman G, Sasisekharan R. 2001. Distinct heparan sulfate glycosaminoglycans are responsible for mediating Fibroblast Growth Factor-2 biological activity though different Fibroblast Growth Factor Receptors. Faseb J 15(8):1422-1424.
Billottet C, Janji B, Thiery J P, Jouanneau J. 2002. Rapid tumor development and potent vascularization are independent events in carcinoma producing FGF-1 or FGF-2. Oncogene 21(53):8128-8139.
Blackhall F H, Merry C L, Davies E J, Jayson G C. 2001. Heparan sulfate proteoglycans and cancer. Br J Cancer 85(8):1094-1098.
Bossennec V, Petitou M, Perly B. 1990. 1H-n.m.r. investigation of naturally occurring and chemically oversulphated dermatan sulphates. Identification of minor monosaccharide residues. Biochem J 267(3):625-630.
Fairbrother W J, Champe M A, Christinger H W, Keyt B A, Starovasnik M A. 1998. Solution structure of the heparin-binding domain of vascular endothelial growth factor. Structure 6(5):637-648.
Fernig D G, Gallagher J T. 1994. Fibroblast growth factors and their receptors: an information network controlling tissue growth, morphogenesis and repair. Prog Growth Factor Res 5(4):353-377.
Ferrara N, Gerber H P, LeCouter J. 2003. The biology of VEGF and its receptors. Nat Med 9(6):669-676.
Finch P W, Rubin J S, Miki T, Ron D, Aaronson S A. 1989. Human KGF is FGF-related with properties of a paracrine effector of epithelial cell growth. Science 245(4919):752-755.
Finch, P. W., and Cheng, A. L. (1999) “Analysis of the cellular basis of keratinocyte growth factor overexpression in inflammatory bowel disease.” Gut 45(6):848-55.
Givol D, Yayon A. 1992. Complexity of FGF receptors: genetic basis for structural diversity and functional specificity. Faseb J 6:3362-3369.
Herr A B, Ornitz D M, Sasisekharan R, Venkataraman G, Waksman G. 1997. Heparin-induced self-association of fibroblast growth factor-2. Evidence for two oligomerization processes. J Biol Chem 272(26):16382-16389.
Iozzo R V, San Antonio J D. 2001. Heparan sulfate proteoglycans: heavy hitters in the angiogenesis arena. Journal of Clinical Investigation 108(3):349-355.
Jackson M W, Roberts J S, Heckford S E, Ricciardelli C, Stahl J, Choong C, Horsfall D J, Tilley W D. 2002. A potential autocrine role for vascular endothelial growth factor in prostate cancer. Cancer Res 62(3):854-859.
Jameson J, Ugarte K, Chen N, Yachi P, Fuchs E, Boismenu R, Havran W L. 2002. A role for skin gammadelta T cells in wound repair. Science 296(5568):747-749.
Joukov V, Pajusola K, Kaipainen A, Chilov D, Lahtinen I, Kulk E, Saksela O, Kalkkinen N, Alitalo K. 1996. A novel vascular endothelial growth factor, VEGF-C, is a ligand for the Flt4 (VEGFR-3) and KDR (VEGFR-2) receptor tyrosine kinases. Embo J 15(2):290-298.
Kan M, Wu X, Wang F, McKeehan W L. 1999. Specificity for fibroblast growth factors determined by heparan sulfate in a binary complex with the receptor kinase. J Biol Chem 274(22):15947-15952.
Kawashima H, Atarashi K, Hirose M, Hirose J, Yamada S, Sugahara K, Miyasaka M. 2002. Oversulfated chondroitin/dermatan sulfates containing GlcAbeta1/IdoAalpha1-3GalNAc(4,6-O-disulfate) interact with L- and P-selectin and chemokines. J Biol Chem 277(15):12921-12930.
Kubo H, Cao R, Brakenhielm E, Makinen T, Cao Y, Alitalo K. 2002. Blockade of vascular endothelial growth factor receptor-3 signaling inhibits fibroblast growth factor-2-induced lymphangiogenesis in mouse cornea. Proc Natl Acad Sci USA 99(13):8868-8873.
Ornitz D M, Xu J, Colvin J S, McEwen D G, MacArthur C A, Coulier F, Gao G, Goldfarb M. 1996. Receptor specificity of the fibroblast growth factor family. J Biol Chem 271(25):15292-15297.
Ostrovsky O, Berman B, Gallagher J, Mulloy B, Fernig D G, Delehedde M, Ron D. 2002. Differential effects of heparin saccharides on the formation of specific fibroblast growth factor (FGF) and FGF receptor complexes. J Biol Chem 277(4):2444-2453.
Ozen M, Giri D, Ropiquet F, Mansukhani A, Ittmann M. 2001. Role of fibroblast growth factor receptor signaling in prostate cancer cell survival. J Natl Cancer Inst 93(23): 1783-1790.
Paavonen K, Puolakkainen P, Jussila L, Jahkola T, Alitalo K. 2000. Vascular endothelial growth factor receptor-3 in lymphangiogenesis in wound healing. Am J Pathol 156(5):1499-1504.
Penc S F, Pomahac B, Winkler T, Dorschner R A, Eriksson E, Herndon M, Gallo R L. 1998. Dermatan sulfate released after injury is a potent promoter of fibroblast growth factor-2 function. J Biol Chem 273(43):28116-28121.
Qi J H, Matsumoto T, Huang K, Olausson K, Christofferson R, Claesson-Welsh L. 1999. Phosphoinositide 3 kinase is critical for survival, mitogenesis and migration but not for differentiation of endothelial cells. Angiogenesis 3(4):371-380.
Raman R, Venkataraman G, Ernst S, Sasisekharan V, Sasisekharan R. 2003. Structural specificity of heparin binding in the fibroblast growth factor family of proteins. Proc Natl Acad Sci USA 100(5):2357-2362.
Rapraeger A C. 1993. The coordinated regulation of heparan sulfate, syndecans and cell behavior. Curr Opin Cell Biol 5:844-853.
Ray, P., Devaux, Y., Stolz, D. B., Yarlagadda, M., Watkins, S. C., Lu, Y., Chen, L., Yang, X. F., and Ray, A. (2003) “Inducible expression of keratinocyte growth factor (KGF) in mice inhibits lung epithelial cell death induced by hyperoxia.” Proc Natl Acad Sci USA 100(10):6098-103.
Robinson C J, Stringer S E. 2001. The splice variants of vascular endothelial growth factor (VEGF) and their receptors. J Cell Sci 114(Pt 5):853-865.
Ropiquet, F., Giri, D., Lamb, D. J., and Ittmann. M. (1999) “FGF7 and FGF2 are increased in benign prostatic hyperplasia and are associated with increased proliferation.” J Urol 162(2):595-9.
Sannes P L, Khosla J, Li C M, Pagan 1. 1998. Sulfation of extracellular matrices modifies growth factor effects on type II cells on laminin substrata. Am J Physiol 275(4 Pt 1):L701-708.
Sasisekharan R, Shriver Z, Venkataraman G, Narayanasami U. 2002. Roles of heparan-sulphate glycosaminoglycans in cancer. Nat Rev Cancer 2(7):521-528.
Sleeman M, Fraser J, McDonald M, Yuan S, White D, Grandison P, Kumble K, Watson J D, Murison J G. 2001. Identification of a new fibroblast growth factor receptor, FGFR5. Gene 271(2):171-182.
Sperinde G V, Nugent M A. 2000. Mechanisms of fibroblast growth factor 2 intracellular processing: a kinetic analysis of the role of heparan sulfate proteoglycans. Biochemistry 39(13):3788-3796.
Stacker S A, Caesar C, Baldwin M E, Thornton G E, Williams R A, Prevo R, Jackson D G, Nishikawa S, Kubo H, Achen M G. 2001. VEGF-D promotes the metastatic spread of tumor cells via the lymphatics. Nat Med 7(2):186-191.
Steiling, H., Muhlbauer, M., Bataille, F., Scholmerich, J., Werner, S., and Hellerbrand, C. (2004) “Activated hepatic stellate cells express keratinocyte growth factor in chronic liver disease.” Am J Pathol 165(4):1233-41.
Strowski M Z, Cramer T, Schafer G, Juttner S, Walduck A, Schipani E, Kemmner W, Wessler S, Wunder C, Weber M, Meyer T F, Wiedenmann B, Jons T, Naumann M, Hocker M. 2003. Helicobacter pylori stimulates host vascular endothelial growth factor-A (vegf-A) gene expression via MEK/ERK-dependent activation of Sp1 and Sp3. Faseb J.
Trowbridge J M, Gallo R L. 2002. Dermatan sulfate: new functions from an old glycosaminoglycan. Glycobiology 12(9):117R-125R.
Trowbridge J M, Rudisill J A, Ron D, Gallo R L. 2002. Dermatan sulfate binds and potentiates activity of keratinocyte growth factor (FGF-7). J Biol Chem 277(45):42815-42820.
Veikkola T, Karkkainen M, Claesson-Welsh L, Alitalo K. 2000. Regulation of angiogenesis via vascular endothelial growth factor receptors. Cancer Res 60(2):203-212.
Wu W, Shu X, Hovsepyan H, Mosteller R D, Broek D. 2003. VEGF receptor expression and signaling in human bladder tumors. Oncogene 22(22):3361-3370.
Ye S, Luo Y, Lu W, Jones R B, Linhardt R J, Capila I, Toida T, Kan M, Pelletier H, McKeehan W L. 2001. Structural basis of interaction of FGF-1, FGF-2, and FGF-7 with different heparan sulfate motifs. Biochemistry 40:14429-14439.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

I/We claim:

1. A method of modulating an activity of a fibroblast growth factor (FGF), comprising:
contacting the FGF with a composition comprising a highly sulfated glycosaminoglycan (GAG), wherein the highly sulfated GAG is in an amount effective to modulate the activity of the FGF, and wherein the highly sulfated GAG is a highly sulfated chondroitin sulfate (CS) or a highly sulfated dermatan sulfate (DS).

2. The method of claim 1, wherein the highly sulfated GAG is an oversulfated dermatan sulfate (DS).

3. The method of claim 2, wherein at least 40% of the disaccharides of the oversulfated DS are either di- or tri-sulfated.

4-7. (canceled)

8. The method of claim 1, wherein the highly sulfated GAG is a highly sulfated chondroitin sulfate (CS).

9. The method of claim 8, wherein at least 40% of the disaccharides of the highly sulfated CS are either di- or tri-sulfated.

10-13. (canceled)

14. The method of claim 1, wherein the highly sulfated CS is chondroitin sulfate D or chondroitin sulfate E.

15. The method of claim 1, wherein the FGF is FGF1, FGF2 or FGF7.

16. The method of claim 1, wherein the activity of the FGF is increased.

17. The method of claim 1, wherein the activity of a vascular endothelial growth factor (VEGF) is also modulated.

18. The method of claim 17, wherein the activity of the VEGF is increased.

19-25. (canceled)

26. The method of claim 17, wherein the VEGF is VEGF-A, VEGF-C or VEGF-D.

27. The method of claim 26, wherein the VEGF is VEGF₁₂₀, VEGF₁₆₄ or VEGF₁₈₈.

28. The method of claim 26, wherein the VEGF is VEGF₁₂₁, VEGF₁₄₅, VEGF₁₆₅, VEGF₁₈₉ or VEGF₂₀₆.

29-48. (canceled)

49. A method of modulating an activity of a VEGF, comprising:
contacting the VEGF with a composition comprising a highly sulfated GAG, wherein the highly sulfated GAG is in an amount effective to modulate the activity of the VEGF, and wherein the highly sulfated GAG is a highly sulfated CS or a highly sulfated DS.

50-102. (canceled)

103. A method of producing an oversulfated DS or oversulfated CS, comprising:
obtaining a fragment of the DS or CS, and
oversulfating the fragment.

104-114. (canceled)

115. A composition, comprising:
the oversulfated DS or oversulfated CS produced by the method of claim 103.

116-117. (canceled)

118. A composition, comprising:
a highly sulfated DS, wherein at least 40% of the disaccharides are ΔDi 4S,6S.

119-124. (canceled)

125. A method of modulating an activity of a FGF, comprising:
contacting the FGF with the composition of claim 115.

126. (canceled)

127. A method of modulating an activity of a VEGF, comprising:
contacting the VEGF with the composition of claim 115.

128. (canceled)

129. A method of modulating an activity of a FGF and an activity of a VEGF, comprising:
contacting the FGF and VEGF with the composition of claim 115.

130. (canceled)
